(19) United States
(12) Patent Application Publication
(10) Pub. No.: US 2011/0039693 A1
(43) Pub. Date: Feb. 17, 2011

(54) Pyrimidylmethyl Sulfonamide Compounds

(75) Inventors: Jan Klaas Lohmann, Mannheim (DE); Alice Glaettu, Frankfurt (DE); Wassilios Grammenos, Ludwigshafen (DE); Jurith Montag, Limburgerhof (DE); Bernd Mueller, Frankenthal (DE); Marianna Vrettou-Schultes, Mannheim (DE); Jens Renner, Bad Duerkheim (DE); Sarah Ulmschneider, Bad Duerkheim (DE); Michael Rack, Eppelheim (DE); Jochen Dietz, Karlsruhe (DE)

Correspondence Address: BRINKS, HOFER, GILSON & LIONE, P.O. BOX 110285, RESEARCH TRIANGLE PARK, NC, 27709, US

(73) Assignee: BASF SE, Ludwigshafen (DE)

(21) Appl. No.: 12/867,548

(22) PCT Filed: Feb. 10, 2009
(86) PCT No.: PCT/EP09/51500

§ 371 Date: Oct. 8, 2010

(30) Foreign Application Priority Data
Feb. 15, 2008 (EP) 08101694.1

(51) Int. Cl. A01N 25/26; C07D 401/12; C07D 403/12; A01N 43/54; A01N 43/58; A01P 3/00

(52) U.S. Cl. 504/100; 544/316; 544/333; 544/296; 544/238; 514/274; 514/256; 514/252.02

(57) ABSTRACT

The present invention relates to pyrimidin-4-ylmethyl-sulfonamides of formula (I) wherein R^{a}, n, R, A, Y and Het are as defined in the claims and to the N-oxides, and salts thereof and their use for combating harmful fungi, and also to compositions and seed comprising at least one such compound. The invention also relates to a process for preparing these compounds.

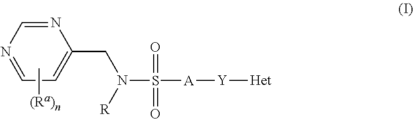

The present invention relates to novel pyrimidin-4-ylmethyl-sulfonamide compounds and the N-oxides, and salts thereof and their use for combating harmful fungi, and also to compositions and seed comprising at least one such compound.

WO 05/033081 describes pyridin-4-ylmethyl sulfonamide compounds. The European non-published application 07122415.8 describes pyridin-4-ylmethyl sulfonamide compounds of formula

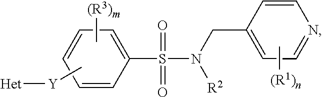

wherein Het is an optionally substituted 5- or 6-membered heteroaryl and Y is selected from —O—, —O—CH₂—, —CH₂—O—S—, —S(═O)—, —S(═O)₂— and —N(Rⁿ)—, wherein Rⁿ is hydrogen or C₁-C₄-alkyl. The compounds described in WO 05/033081 and the European non-published application 07122415.8 are suitable for use as crop protection agents against harmful fungi.

WO 08/062011 describes pyrimidin-4-ylmethyl sulfonamide compounds of formula

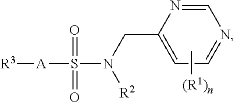

and their use as crop protection agents. Compounds in which A is phenylene or a 5- or 6-membered heteroarendiyl and R³ is a 5- or 6-membered heteroaryloxy or heteroarylthio are generally covered by this patent application. However, there is no single compound disclosed in which A is phenylene or a 5- or 6-membered heteroarenediyl and R³ is a 5- or 6-membered heteroaryloxy or heteroarylthio.

However, with respect to their fungicidal activity, the action of the compounds disclosed is not always completely satisfactory. Based on this, it was an object of the present invention to provide compounds having improved action and/or a broadened activity spectrum against harmful fungi.

This object is, surprisingly, achieved by pyrimidin-4-ylmethyl-sulfonamide compounds of formula I as defined herein and by the N-oxides and their salts, in particular the agriculturally salts.

The compounds of the formula I differ from those known from the abovementioned publications by the combination of the pyrimidin-4-ylmethyl group with the specific sulfonic acid substituent A-Y-Het.

Accordingly, the present invention relates to compounds of formula I

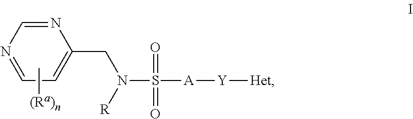

wherein:
n indicates the number of substituents R^{a} on the pyrimidine ring and n is 0, 1, 2 or 3;
R^{a} is halogen, CN, NH₂, NO₂, OH, SH, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylamino, di(C₁-C₄-alkyl)amino, C₁-C₄-alkoxy-C₁-C₄-alkyl, C₂-C₄-alkenyl, C₂-C₄-alkynyl, C₃-C₈-cycloalkyl or C₁-C₄-alkyl-C₃-C₈-cycloalkyl; and/or
   two radicals R^{a} that are bound to adjacent ring member atoms of the pyrimidine ring may form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted or carries 1, 2, 3 or 4 identical or different radicals selected from the group consisting of halogen, CN, C₁-C₄-alkyl, C₁-C₄-alkoxy, C₁-C₄-haloalkyl and C₁-C₄-haloalkoxy;
   it being possible for n=2 or 3 that R^{a} are identical or different;
R is hydrogen, C₁-C₄-alkyl, C₁-C₄-haloalkyl, C₁-C₄-alkoxy, C₁-C₄-haloalkoxy, C₁-C₄-alkylamino, di(C₁-C₄-alkyl)amino, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₃-C₈-cycloalkyl, C₁-C₄-alkyl-C₃-C₈-cycloalkyl or benzyl wherein the phenyl moiety of benzyl is unsubstituted or carries 1, 2, 3, 4, or 5 substituents selected from the group consisting of cyano, halogen, C₁-C₄-alkyl, C₁-C₄-haloalkyl, C₁-C₄-alkoxy, C₁-C₄-haloalkoxy, (C₁-C₄-alkoxy)carbonyl and di(C₁-C₄-alkyl)aminocarbonyl,
A is phenylene or a 5- or 6-membered heteroarenediyl, wherein the ring member atoms of the heteroarenediyl include besides carbon atoms 1, 2, 3 or 4 heteroatoms selected from the group of N, O and S, and wherein the aforementioned divalent radicals are unsubstituted or carry 1, 2, 3 or 4 identical or different groups R^{b}:
   R^{b} is halogen, CN, NO₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, (C₁-C₄-alkyl)carbonyl, (C₁-C₄-alkoxy)carbonyl, C₁-C₄-alkylamino, di(Ci-C₄-alkyl)amino, (C₁-C₄-alkyl)aminocarbonyl and di(C₁-C₄-alkyl)aminocarbonyl;
Y is a divalent group selected from —O—, —C(═O)—, —O—CH₂—, —CH₂—O—, —S—, —S(═O)—, —S(═O)₂—, C₁-C₄-alkanediyl, —N(R^{π})— and —C(NOR^{π})—, wherein R^{π} is hydrogen or C₁-C₄-alkyl;
Het is a 5- or 6-membered heteroaryl, wherein the ring member atoms of the heteroaryl include besides carbon atoms 1, 2, 3 or 4 heteroatoms selected from the group of N, O and S and wherein the heteroaryl is unsubstituted or carries 1, 2, 3 or 4 identical or different groups R^{c}:
   R^{c} is halogen, CN, NO₂, NH₂, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-halo-alkoxy-C₁-C₄-alkyl, C₂-C₆-alkenyl, C₂-C₆-alkynyl, C(═O)R′, C(═NOR|)R′″, C₃-C₈-cycloalkyl, C₁-C₄-alkyl-C₃-C₈-cycloalkyl, phenyl, phenoxy, phenoxy-C₁-C₄-alkyl or a 5- or 6-membered heteroaryl, wherein the ring member atoms of the heteroaryl include besides carbon atoms 1, 2, 3 or 4 heteroatoms selected from the group of N, O and S, and wherein the aforementioned cyclic radicals are unsubstituted or carry 1, 2, 3 or 4 identical or different substituents R^{d}:
      R′ is hydrogen, NH₂, C₁-C₄-alkyl, C₁-C₄-haloalkyl, C₂-C₄-alkenyl, C₂-C₄-alkynyl, C₁-C₄-alkoxy, C₁-C₄-alkoxy-C₁-C₄-alkoxy, C₁-C₄-haloalkoxy, C₁-C₄-alkylamino or di(C₁-C₄-alkyl)amino;
      R″ is hydrogen, C₁-C₄-alkyl, C₁-C₄-haloalkyl, C₂-C₄-alkenyl, C₂-C₄-alkynyl or C₁-C₄-alkoxy-C₁-C₄-alkyl,
      R′″ is hydrogen or C₁-C₄-alkyl;
      R^{d} is halogen, CN, C₁-C₄-alkyl, C₁-C₄-haloalkyl, C₁-C₄-alkoxy or C₁-C₄-haloalkoxy;
   and/or two radicals R^{c} that are bound to adjacent ring member atoms of the Het group may form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted or carries 1, 2, 3 or 4 identical or different radicals groups R^{e}:
      R^{e} is halogen, CN, C₁-C₄-alkyl, C₁-C₄-haloalkyl, C₁-C₄-alkoxy or C₁-C₄-haloalkoxy;
         and the N-oxides and the agriculturally acceptable salts of the compounds of formula I, and of compositions comprising compounds of formula I, for combating harmful fungi.

The present invention furthermore relates to processes for preparing the compounds I.

The present invention furthermore relates to intermediates such as compounds of formulae II, Ill, IV and V.

The present invention furthermore relates to an agrochemical composition which comprises a solid or liquid carrier and at least one compound of formula I or an N-oxide or an agriculturally acceptable salt thereof.

The compounds of the present invention are useful for combating harmful fungi. Therefore the present invention furthermore relates to a method for combating harmful fungi, which process comprises treating the fungi or the materials, plants, the soil or seeds to be protected against fungal attack, with an effective amount of at least one compound of formula I or of an N-oxide or an agriculturally acceptable salt thereof.

Furthermore, the present invention also relates to seed comprising a compound of formula I, or an N-oxide or an agriculturally acceptable salt thereof, in an amount of from 0.1 g to 10 kg per 100 kg of seed.

Depending on the substitution pattern, the compounds of formula I and their N-oxides may have one or more centers of chirality, in which case they are present as pure enantiomers or pure diastereomers or as enantiomer or diastereomer mixtures. Both, the pure enantiomers or diastereomers and their mixtures are subject matter of the present invention.

The compounds of formula I can be present in different crystal modifications whose biological activity may differ. They also form part of the subject matter of the present invention.

Agriculturally useful salts of the compounds I encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound I with an acid of the corresponding anion, preferably of hydrochloric acid, hydrobromic acid, sulfuric acid, phosphoric acid or nitric acid.

The compounds of formula I can be present in atropisomers arising from restricted rotation about a single bond of asymmetric groups. They also form part of the subject matter of the present invention.

In respect of the variables, the embodiments of the intermediates correspond to the embodiments of the compounds of formula I.

The term “compounds I” refers to compounds of formula I. Likewise, the term “compounds I.1” refers to compounds of formula I.1.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₄-alkyl” refers to a straight-chained or branched saturated hydrocarbon group having 1 to 4 carbon atoms, for example methyl, ethyl, propyl, 1-methylethyl, butyl, 1-methylpropyl, 2-methylpropyl, and 1,1-dimethylethyl. Likewise, the term “C₁-C₆-alkyl” refers to a straight-chained or branched saturated hydrocarbon group having 1 to 6 carbon atoms.

The term “C₁-C₄-haloalkyl” refers to a straight-chained or branched alkyl group having 1 to 4 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. Likewise, the term “C₁-C₆-haloalkyl” refers to a straight-chained or branched alkyl group having 1 to 6 carbon atoms.

The term “C₁-C₄-alkoxy” refers to a straight-chain or branched alkyl group having 1 to 4 carbon atoms (as defined above) which is bonded via an oxygen, at any position in the alkyl group, for example methoxy, ethoxy, n-propoxy, 1-methylethoxy, butoxy, 1-methyl [symbol] propoxy, 2-methylpropoxy or 1,1-dimethylethoxy. Likewise, the term “C₁-C₄-alkoxy” refers to a straight-chain or branched alkyl group having 1 to 6 carbon atoms.

The term “C₁-C₄-haloalkoxy” refers to a C₁-C₄-alkoxy group as defined above, wherein some or all of the hydrogen atoms may be replaced by halogen atoms as mentioned above, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloro [symbol] ethoxy, OC₂F₅, 2-fluoropropoxy, 3-fluoropropoxy, 2,2-difluoropropoxy, 2,3-difluoro [symbol] propoxy, 2-chloropropoxy, 3-chloropropoxy, 2,3-dichloropropoxy, 2-bromo [symbol] propoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromo [symbol] ethoxy, 4-fluorobutoxy, 4-chlorobutoxy, 4-bromobutoxy or nonafluorobutoxy. Likewise, the term “C₁-C₆-haloalkoxy” refers to a C₁-C₆-alkoxy group as defined above, wherein some or all of the hydrogen atoms may be replaced by halogen atoms as mentioned above.

The term “C₁-C₄-alkoxy-C₁-C₄-alkyl” refers to alkyl having 1 to 4 carbon atoms (as defined above), wherein one hydrogen atom of the alkyl radical is replaced by a C₁-C₄-alkoxy group (as defined above). Likewise, the term “C₁-C₆-alkoxy-C₁-C₄-alkyl” refers to alkyl having 1 to 4 carbon atoms (as defined above), wherein one hydrogen atom of the alkyl radical is replaced by a C₁-C₆-alkoxy group (as defined above).

The term “C₁-C₄-haloalkoxy-C₁-C₄-alkyl” refers to alkyl having 1 to 4 carbon atoms (as defined above), wherein one hydrogen atom of the alkyl radical is replaced by a C₁-C₄-haloalkoxy group (as defined above). Likewise, the term “C₁-C₆-haloalkoxy-C₁-C₄-alkyl” refers to alkyl having 1 to 4 carbon atoms (as defined above), wherein one hydrogen atom of the alkyl radical is replaced by a C₁-C₆-alkoxy group (as defined above).

The term “C₁-C₄-alkoxy-C₁-C₄-alkoxy” refers to an C₁-C₄-alkoxy-C₁-C₄-alkyl group (as defined above), which is bonded via an oxygen atom to the remainder of the molecule.

The term “C₁-C₄-alkylthio” as used herein refers to straight-chain or branched alkyl groups having 1 to 4 carbon atoms (as defined above) bonded via a sulfur atom, at any position in the alkyl group, for example methylthio, ethylthio, propylthio, isopropylthio, and n butylthio. Likewise, the term “C₁-C₆-alkylthio” as used herein refers to straight-chain or branched alkyl groups having 1 to 6 carbon atoms (as defined above) bonded via a sulfur atom. Accordingly, the terms “C₁-C₄-haloalkylthio” and “C₁-C₆-haloalkylthio” as used herein refer to straight-chain or branched haloalkyl groups having 1 to 4 or 1 to 6 carbon atoms (as defined above) bonded through a sulfur atom, at any position in the haloalkyl group.

The terms “C₁-C₄-alkylsulfinyl” or “C₁-C₆-alkylsulfinyl” refer to straight-chain or branched alkyl groups having 1 to 4 or 1 to 6 carbon atoms (as defined above) bonded through a —S(═O)— moiety, at any position in the alkyl group, for example methylsulfinyl and ethylsulfinyl, and the like. Accordingly, the terms “C₁-C₄-haloalkylsulfinyl” and “C₁-C₆-haloalkylsulfinyl”, respectively, refer to straight-chain or branched haloalkyl groups having 1 to 4 and 1 to 6 carbon atoms (as defined above), respectively, bonded through a —S(═O)— moiety, at any position in the haloalkyl group.

The terms “C₁-C₄-alkylsulfonyl” and “C₁-C₆-alkylsulfonyl”, respectively, refer to straight-chain or branched alkyl groups having 1 to 4 and 1 to 6 carbon atoms (as defined above), respectively, bonded through a —S(═O)₂— moiety, at any position in the alkyl group, for example methylsulfonyl. Accordingly, the terms “C₁-C₄-haloalkylsulfonyl” and “C₁-C₆-haloalkylsulfonyl”, respectively, refer to straight-chain or branched haloalkyl groups having 1 to 4 and 1 to 6 carbon atoms (as defined above), respectively, bonded through a —S(═O)₂— moiety, at any position in the haloalkyl group.

The term “C₁-C₄-alkylamino” refers to an amino radical carrying one C₁-C₄-alkyl group (as defined above) as substituent, for example methylamino, ethylamino, propylamino, 1-methylethylamino, butylamino, 1-methylpropylamino, 2-methylpropylamino, 1,1-dimethylethylamino and the like. Likewise, the term “C₁-C₆-alkylamino” refers to an amino radical carrying one C₁-C₆-alkyl group (as defined above) as substituent.

The term “di(C₁-C₄-alkyl)amino” refers to an amino radical carrying two identical or different C₁-C₄-alkyl groups (as defined above) as substituents, for example dimethylamino, diethylamino, di-n-propylamino, diisopropylamino, N-ethyl-N-methylamino, N-(n-propyl)-N-methylamino, N-(isopropyl)-N methylamino, N-(n-butyl)-N-methylamino, N-(n-pentyl)-N-methylamino, N-(2-butyl)-N methylamino, N-(isobutyl)-N-methylamino, and the like. Likewise, the term “di(C₁-C₆-alkyl)amino” refers to an amino radical carrying two identical or different C₁-C₆-alkyl groups (as defined above) as substituents.

The term “(C₁-C₄-alkoxy)carbonyl” refers to a C₁-C₄-alkoxy radical (as defined above) which is attached via a carbonyl group.

The term “di(C₁-C₄-alkyl)aminocarbonyl” refers to a di(C₁-C₄)alkylamino radical as defined above which is attached via a carbonyl group.

The term “phenoxy” and refers to a phenyl radical which is attached via an oxygen atom. Likewise, the term “phenoxy-C₁-C₄-alkyl” and refers to a phenoxy radical which is attached via a C₁-C₄-alkyl group (as defined above).

The term “C₂-C₄-alkenyl” refers to a straight-chain or branched unsaturated hydrocarbon radical having 2 to 4 carbon atoms and a double bond in any position, such as ethenyl, 1-propenyl, 2-propenyl(allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl. Likewise, the term “C₂-C₆-alkenyl” refers to a straight-chain or branched unsaturated hydrocarbon radical having 2 to 6 carbon atoms and a double bond in any position.

The term “C₂-C₄-alkynyl” refers to a straight-chain or branched unsaturated hydrocarbon radical having 2 to 4 carbon atoms and containing at least one triple bond, such as ethynyl, 1-propynyl, 2-propynyl (propargyl), 1-butynyl, 2-butynyl, 3-butynyl, 1-methyl-2-propynyl. Likewise, “C₂-C₆-alkynyl” refers to a straight-chain or branched unsaturated hydrocarbon radical having 2 to 6 carbon atoms and at least one triple bond.

The term “C₃-C₈-cycloalkyl” refers to monocyclic saturated hydrocarbon radicals having 3 to 8 carbon ring members, such as cyclopropyl (C₃C₅), cyclobutyl, cyclopentyl, cyclohexyl, cycloheptyl or cyclooctyl.

The term “C₁-C₄-alkyl-C₃-C₈-cycloalkyl” refers to a cycloalkyl radical having 3 to 8 carbon atoms (as defined above), wherein one hydrogen atom of the cycloalkyl radical is replaced by a C₁-C₄-alkyl group (as defined above).

The term “5-, 6- or 7-membered carbocycle” is to be understood as meaning both saturated or partially unsaturated carbocycles having 5, 6 or 7 ring members as well as phenyl. Examples for non-aromatic rings include cyclopentyl, cyclopentenyl, cyclopentadienyl, cyclohexyl, cyclohexenyl, cyclohexadienyl, cycloheptyl, cycloheptenyl, cycloheptadienyl, and the like.

The term “5-, 6-, or 7-membered heterocycle” wherein the ring member atoms of the heterocycle include besides carbon atoms 1, 2, 3 or 4 heteroatoms selected from the group of N, O and S, is to be understood as meaning both saturated and partially unsaturated as well as aromatic heterocycles having 5, 6 or 7 ring atoms.

### Examples Include:

saturated and partially unsaturated 5-, 6-, or 7-membered heterocycle wherein the ring member atoms of the heterocycle include besides carbon atoms 1, 2 or 3 heteroatoms selected from the group of N, O and S, and which is saturated or partially unsaturated, for example pyrrolidin-2-yl, pyrrolidin-3-yl, tetrahydrofuran-2-yl, tetrahydrofuran-3-yl, tetrahydrothien-2-yl, tetrahydrothien-3-yl, 1,3-dioxolan-4-yl, isoxazolidin-3-yl, isoxazolidin-4-yl, isoxazolidin-5-yl, isothiazolidin-3-yl, isothiazolidin-4-yl, isothiazolidin-5-yl, pyrazolidin-3-yl, pyrazolidin-4-yl, pyrazolidin-5-yl, oxazolidin-2-yl, oxazolidin-4-yl, oxazolidin-5-yl, thiazolidin-2-yl, thiazolidin-4-yl, thiazolidin-5-yl, imidazolidin-2-yl, imidazolidin-4-yl, 2-pyrrolin-2-yl, 2-pyrrolin-3-yl, 3-pyrrolin-2-yl, 3-pyrrolin-3-yl, piperidin-2-yl, piperidin-3-yl, piperidin-4-yl, 1,3-dioxan-5-yl, tetrahydropyran-2-yl, tetrahydropyran-4-yl, tetrahydrothien-2-yl, hexahydropyridazin-3-yl, hexahydropyridazin-4-yl, hexahydropyrimidin-2-yl, hexahydropyrimidin-4-yl, 5-hexahydropyrimidinyl and piperazin-2-yl;
5-membered heteroaryl (heteroaromatic radical), wherein the ring member atoms of the heteroaryl include besides carbon atoms 1, 2 or 3 heteroatoms selected from the group of N, O and S, for example pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl;
6-membered heteroaryl (heteroaromatic radical), wherein the ring member atoms of the heteroaryl include besides carbon atoms 1, 2 or 3 heteroatoms selected from the group of N, O and S, for example pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl.

The terms “C₁-C₄-alkanediyl” and “C₁-C₈-alkanediyl” refer to divalent, branched, or straight-chain saturated hydrocarbon radicals having 1to 4 and 1 to 8 carbon atoms respectively, derived by the removal of one hydrogen atom from each of two different carbon atoms of a parent alkane, or by the removal of two hydrogen atoms from a single carbon atom of a parent alkane, for example, methanediyl, ethan-1,1-diyl, ethan-1,2-diyl, propan-1,1-diyl, propan-1,2-diyl, propan-2,2-diyl, propan-1,3-diyl, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, and the like.

The term “C₁-C₈-haloalkanediyl” refers to a divalent, branched, or straight-chain saturated hydrocarbon group having 1 to 8 carbon atoms, as defined above, wherein some or all of the hydrogen atoms in these groups may be replaced by halogen atoms as mentioned above.

The term “C₂-C₈-alkenediyl” refers to a divalent, branched, or straight-chain unsaturated hydrocarbon group having 2 to 8 carbon atoms, derived by the removal of one hydrogen atom from each of two different carbon atoms of a parent C₂-C₈-alkene, or by the removal of two hydrogen atoms from a single carbon atom of a parent C₂-C₈-alkene, for example, ethen-1,2-diyl, ethen-1,1-diyl, prop-1-en-1,1-diyl, prop-2-en-1,2-diyl, prop-1-en-1,3-diyl, propen-3,3-diyl, propen-2,2-diyl, but-2-en-1,4-diyl and the like.

The term “C₂-C₈-haloalkenediyl” refers to a divalent, branched, or straight-chain unsaturated hydrocarbon group having 2 to 8 carbon atoms, as defined above, wherein some or all of the hydrogen atoms in these groups may be replaced by halogen atoms as mentioned above.

The term “C₂-C₈-alkynediyl” refers to a divalent, branched, or straight-chain unsaturated hydrocarbon radical having 2 to 8 carbon atoms, derived by the removal of one hydrogen atom from each of two different carbon atoms of a parent C₂-C₈-alkyne, or by the removal of two hydrogen atoms from a single carbon atom of a parent C₂-C₈-alkyne, for example, prop-2-yn-1,1-diyl, prop-2-yn-1,3-diyl, prop-1-yn-1,3-diyl, but-1-yn-1,3-diyl, but-1-yn-1,4-diyl, but-2-yn-1,4-diyland the like.

The term “C₂-C₈-haloalkynediyl” refers to a divalent, branched, or straight-chain unsaturated hydrocarbon radical having 2 to 8 carbon, as defined above, wherein some or all of the hydrogen atoms in these groups may be replaced by halogen atoms as mentioned above.

As used herein, the term “C₃-C₈-cycloalkylene” refers to a divalent radical derived from a C₃-C₈-cycloalkyl group (as defined above) that has two points of attachment. Likewise, the term “C₃-C₈-cycloalkenylene” refers to a divalent radical derived from a C₃-C₈-cycloalkenyl group (as defined above) that has two points of attachment. Accordingly, the term “heterocyclylene” refers to a heterocyclyl group (as defined above) that has two points of attachment.

The term “phenylene” refers to 1,2-phenylene (o-phenylene), 1,3-phenylene (m-phenylene) and 1,4-phenylen (p-phenylene).

Furthermore, the term “5- or 6-membered heteroarenediyl” refers to a divalent radical derived from an aromatic heteroaryl (as defined above) having two points of attachment. Examples of heteroarenediyl radicals are, for example, divalent radicals derived from pyridine, pyrimidine, pyridazine, 1,2,3-triazine, 1,2,4-triazine, 1,2,3,4-tetrazine, furan, thiophene, pyrrole, thiazole, thiadiazole, pyrazole, imidazole, triazole, tetrazole, oxazole, isoxazole, isothiazole, oxadiazole and the like. The aforementioned groups can be C-attached or N-attached where such is possible. For example, a group derived from pyrrole, imidiazole or pyrazole can be N-attached or C-attached.

The term “two radicals R^{a} that are bound to adjacent ring member atoms of the pyrimidine ring may form together with said ring member atoms a fused cycle” refers to a condensed bicyclic ring system, wherein the pyrimidine ring carries a fused-on 5-, 6- or 7-membered carbocyclic or heterocyclic ring.

The term “two radicals Rc that are bound to adjacent ring member atoms of the Het group may form together with said ring member atoms a fused cycle” refers to a condensed bicyclic ring system, wherein the 5- or 6-membered heteroaryl, carry a fused-on 5-, 6- or 7-membered carbocyclic or heterocyclic ring.

As regards the fungicidal activity of the compounds I, preference is given to those compounds I and where applicable also to compounds of all sub-formulae provided herein, for example formulae I.1 and I.1a and formulae I.A to I.K and to the intermediates, for example compounds IX.a, wherein the substituents and variables (R, A, Y, Het, R^{a}, R^{b}, R^{c}, R^{d}, R^{e}, R′, R″, R′″ and n) have independently of each other or more preferably in combination the following meanings:

One embodiment relates to compounds I, wherein n is 0 and the pyrimidine ring is unsubstituted. Another embodiment relates to compounds I, wherein n is 1 or 2 and the pyrimidine ring of compounds I carries 1 or 2 radicals R^{a}. A further embodiment relates to compounds I, wherein n is 2 and the pyrimidine ring of compounds I carries two radicals R^{a}. A further embodiment relates to compounds I, wherein n is 1 and the pyrimidine ring of compounds I carries one radical R^{a}. If n is 1, in a specific embodiment, R^{a} is bound to the 2-position of the pyrimidine ring. If n is 1, in a specific embodiment, R^{a} is bound to the 5-position of the pyrimidine ring. If n is 1, in a specific embodiment, R^{a} is bound to the 6-position of the pyrimidine ring.

A further embodiment relates to compounds I, wherein two radicals R^{a} that are bound to adjacent ring member atoms of the pyrimidine ring do not form together with said ring member atoms any fused cycle.

Preferably, R^{a} is halogen, CN, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₂-C₄-alkynyl, C₁-C₄-alkoxy-C₁-C₄-alkyl, C₃-C₈-cycloalkyl or C₁-C₄-alkyl-C₃-C₈-cycloalkyl. Even more preferably, R^{a} is halogen, CN, C₁-C₄-alkyl, C₁-C₄-haloalkyl, C₁-C₄-alkoxy, C₁-C₄-haloalkoxy, C₁-C₄-alkoxy-C₁-C₄-alkyl, C₃-C₈-cycloalkyl or C₁-C₄-alkyl-C₃-C₈-cycloalkyl.

A further embodiment relates to compounds I, wherein R^{a} is selected from F, Cl, Br, OH, SH, CN, C₁-C₂-alkyl, cyclopropyl, CH═CH₂, CCH, ≡C₁-C₂-alkoxy, methylthio, methylamino, dimethylamino, CF₃, CHF₂, OCF₃ and OCHF₂.

A further embodiment relates to compounds I, wherein R^{a} is halogen and preferably selected from fluorine and chlorine and in particular, R^{a} is chlorine.

A further embodiment relates to compounds I, wherein R^{a} is C₁-C₄-alkyl and selected from methyl, ethyl, n-propyl, i-propyl, n-butyl, 1-methyl-propyl, 2-methyl-propyl and 1,1-dimethylethyl, and preferably selected from methyl, ethyl, n-propyl and i-propyl, and in particular, R^{a} is methyl.

A further embodiment relates to compounds I, wherein R^{a} is C₁-C₄-haloalkyl, preferably C₁-haloalkyl, and in particular, R^{a} is trifluormethyl.

A further embodiment relates to compounds I, wherein R^{a} is C₁-C₄-alkoxy and preferably selected from methoxy, ethoxy, n-propyloxy and i-propyloxy, and in particular, R^{a} is methoxy.

A further embodiment relates to compounds I, wherein R^{a} is C₁-C₄-haloalkoxy and specifically halomethoxy, such as difluormethoxy, trifluormethoxy, dichlormethoxy and trichlormethoxy, and haloethoxy, such as 2,2-difluorethoxy, 2,2,2-trifluorethoxy, 2,2dichlorethoxy and 2,2,2-trichlorethoxy, and halo-n-propoxy, halo-i-propoxy, halo-n-butoxy, halo-1-methyl-propoxy, halo-2-methyl-propoxy or halo-1,1-dimethylethoxy.

A further embodiment relates to compounds I, wherein R^{a} is C₃-C₈-cycloalkyl and selected from cyclopropyl, cycobutyl, cyclopentyl, cyclohexyl, cycloheptyl, cyclooctyl, and selected from cyclopropyl, cylopentyl and cyclohexyl, and in particular, R^{a} is cyclopropyl.

A further embodiment relates to compounds I, wherein two radicals R^{a} that are bound to adjacent ring member atoms of the pyrimidine ring form together with said ring member atoms a fused cycle being a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted and carries 1, 2, 3 or 4 identical or different radicals selected from the group consisting of halogen, CN, C₁-C₄-alkyl, C₁-C₄-alkoxy, C₁-C₄-haloalkyl and C₁-C₄-haloalkoxy. In the abovementioned embodiment, the fused cycle is preferably phenyl. In the abovementioned embodiment, the fused cycle is preferably a saturated carbocycle and in particular cyclopentyl. In the abovementioned embodiment, the fused cycle is preferably a partially unsaturated carbocycle, and in particular cyclopentenyl.

Preference is given to compounds I, wherein two radicals R^{a} that are bound to adjacent ring member atoms of the pyrimidine ring form together with said ring member atoms a fused optionally substituted 5-membered heteroaryl. In the abovementioned embodiment, the fused heteroaryl is furanyl. In the abovementioned embodiment, the fused heteroaryl is thienyl. In the abovementioned embodiment, the fused heteroaryl is pyrrolyl.

In one embodiment of the invention, the two radicals R^{a} that are bound to adjacent ring member atoms of the pyrimidine ring form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted.

In a further embodiment, two radicals R^{a} that are bound to adjacent ring member atoms of the pyrimidine ring form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is substituted by 1, 2, 3 or 4 identical or different radicals selected from the group consisting of halogen, CN, C₁-C₄-alkyl, C₁-C₄-alkoxy, C₁-C₄-haloalkyl and C₁-C₄-haloalkoxy.

Specific embodiments relate to compounds I, wherein R^{a1}, R^{a2} and R^{a3} are each independently hydrogen or have one of the definitions specified for R^{a} and wherein the pyrimidyl group carries one of the following combinations of the radicals R^{a1}, R^{a2} and R^{a3} as defined in Table P, which compounds are of formula 1.1

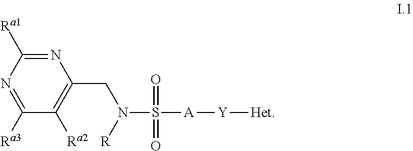

TABLE P

| line | R^{a1} | R^{a2} | R^{a3} |
|---|---|---|---|
| P-1 | OCH₃ | H | H |
| P-2 | OCHF₂ | H | H |
| P-3 | CH₃ | H | H |
| P-4 | H | H | H |

One embodiment relates to compounds I, wherein R is hydrogen, C₁-C₄-alkyl, C₁-C₄-haloalkyl, C₁-C₄-alkoxy or C₁-C₄-haloalkoxy.

Another embodiment relates to compounds I, wherein R is C₁-C₄-alkyl, —CH₂—CH═CH₂ or —CH₂—C≡CH.

A further embodiment relates to compounds I, wherein R is C₁-C₄-alkyl and preferably selected from methyl, ethyl, n-propyl and i-propyl, and in particular, R is methyl.

A further embodiment relates to compounds I, wherein R is hydrogen and R^{a1}, R^{a2} and R^{a1} are each independently hydrogen or have one of the definitions specified for R^{a}, especially those being preferred, which compounds are of formula I.1a

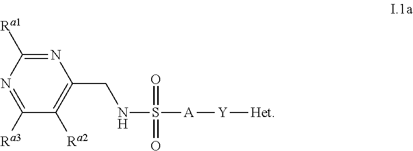

One embodiment of the invention relates to compounds I, wherein A is 1,4-phenylene, which is unsubstituted or carries 1, 2, 3 or 4 identical or different substituents R^{b}, more preferably said 1,4-phenylene is unsubstituted.

Another embodiment relates to compounds I, wherein A is 1,3-phenylene, which is unsubstituted or carries 1, 2, 3 or 4 identical or different substituents R^{b}.

A further embodiment relates to compounds I, wherein A is heteroarenediyl selected from the group consisting of pyridindiyl, pyrimidindiyl, pyridazindiyl, pyrazindiyl, triazindiyl, furandiyl, thiendiyl, pyrroldiyl, pyrazoldiyl, isoxazoldiyl, isothiazoldiyl, imidazoldiyl, oxazoldiyl, thiazoldiyl, triazoldiyl, thiadiazoldiyl, oxadiazoldiyl and tetrazoldiyl, and wherein the 18 last-mentioned radicals are unsubstituted or carry 1, 2 or 3 identical or different substituents R^{b}. If one point of attachment is located on a nitrogen atom of the heteroarenediyl radical, said nitrogen atom is attached either to the sulfur atom of the sulfonamide group or to Y, with the point of attachment to Y being more preferred. In the abovementioned embodiment, A is pyridindiyl. In the abovementioned embodiment, A is pyrimidindiyl. In the abovementioned embodiment, A is pyridazindiyl. In the abovementioned embodiment, A is pyrazindiyl. In the abovementioned embodiment, A is furandiyl. In the abovementioned embodiment, A is thiendiyl. In the abovementioned embodiment, A is pyrroldiyl. In the abovementioned embodiment, A is pyrazoldiyl. In the abovementioned embodiment, A is isoxazoldiyl. In the abovementioned embodiment, A is isothiazoldiyl. In the abovementioned embodiment, A is imidazoldiyl. In the abovementioned embodiment, A is oxazoldiyl. In the abovementioned embodiment, A is thiazoldiyl. In the abovementioned embodiment, A is 1,2,4-triazoldiyl. In the above-mentioned embodiment, A is 1,2,4-thiadiazoldiyl. In the abovementioned embodiment, A is 1,2,4-oxadiazoldiyl.

Amongst compounds I, in which A is a 6-membered heteroarenediyl, particular preference given to those, in which A is pyridindiyl or pyrimidinyl, wherein each of the aforementioned two radicals are unsubstituted or carry 1, 2 or 3 identical or different substituents R^{b}.

Amongst compounds I, in which A is a 6-membered heteroarenediyl, most preference is given to those, in which A is selected from the group consisting of pyridin-2,5-diyl, pyridin-2,6-diyl, pyridin-2,4-diyl, pyridin-3,5-diyl, pyrimidin-2,5-diyl, pyrimidin-2,4-diyl and pyrimidin-4,6-diyl wherein the aforementioned heteroarenediyl radicals are unsubstituted or carry 1, 2, 3 or 4 identical or different substituents R^{b}.

Amongst compounds I, in which A is a 5-membered heteroarenediyl, particular preference given to those, in which A is thiendiyl, thiazoldiyl, oxazoldiyl, pyrazoldiyl or pyridindiyl, wherein each of the aforementioned five radicals are unsubstituted or carry 1, 2 or 3 identical or different substituents R^{b}.

Amongst compounds I, in which A is a 5-membered heteroarenediyl, most preference is given to those, in which A is selected from the group consisting of thien-2,5-diyl, thien-2,4-diyl, thien-3,5-diyl, thiazol-2,5-diyl, thiazol-2,4-diyl, oxazol-2,5-diyl, oxazol-2,4-diyl, pyrazol-3,5-diyl, pyrazol-1,3-diyland pyrazol-1,4-diyl, wherein the aforementioned heteroarenediyl radicals are unsubstituted or carry 1, 2, 3 or 4 identical or different substituents R^{b}.

Particularly preferred embodiments of the invention relate to compounds I, in which A is one of the following radicals A-1 to A-26:

| No. | A |
|---|---|
| A-1 | 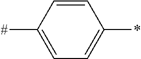 |
| A-2 | 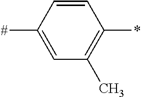 |
| A-3 | 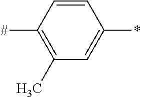 |
| A-4 | 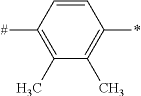 |
| A-5 | 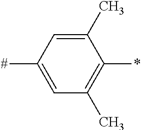 |
| A-6 | 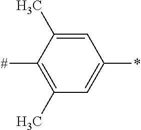 |
| A-7 | 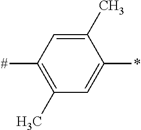 |
| A-8 | 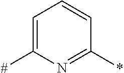 |
| A-9 | 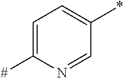 |
| A-10 | 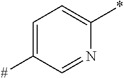 |
| A-11 | 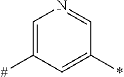 |
| A-12 | 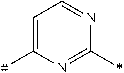 |
| A-13 | 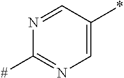 |
| A-14 | 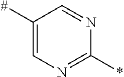 |
| A-15 | 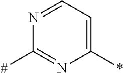 |
| A-16 | 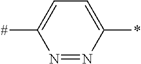 |
| A-17 | 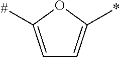 |
| A-18 | 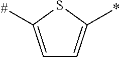 |
| A-19 | 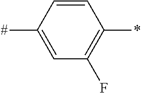 |
| A-20 | 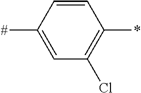 |
| A-21 | 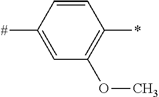 |
| A-22 | 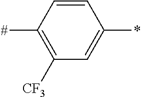 |
| A-23 | 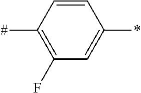 |
| A-24 | 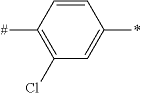 |
| A-25 | 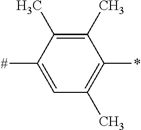 |
| A-26 | 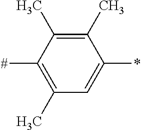 |

wherein # indicates the point of attachment to the sulfur atom of the sufonamide group; and * indicates the point of attachment to Y.

One embodiment of the invention relates to compounds I, wherein the group A of compounds of the formula I carries 1 or 2 radicals R^{b}. In another embodiment of the invention, the group A of compounds I is unsubstituted or carries 1 radical R^{b}. In a further embodiment, the group A is unsubstituted. In a further embodiment, the group A carries 1 radical R^{b}. In a further embodiment, the group A carries 2 radicals R^{b}.

If R^{b} is present, R^{b} is halogen, CN, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, (C₁-C₄-alkyl)carbonyl, (C₁-C₄-alkoxy)carbonyl, C₁-C₄-alkylamino, di(C₁-C₄-alkyl)amino, (C₁-C₄-alkyl)aminocarbonyl or di(C₁-C₄-alkyl)aminocarbonyl. If R^{b} is present, R^{b} is halogen, CN, C₁-C₄-alkyl, C₁-C₄-haloalkyl, C₁-C₄-alkoxy or C₁-C₄-haloalkoxy. R^{b} is present, R^{b} is halogen, CN, C₁-C₄-alkyl, C₁-C₄-haloalkyl, C₁-C₄-alkoxy, C₁-C₄-haloalkoxy, C₁-C₄-alkoxy-C₁-C₄-alkyl, C₃-C₈-cycloalkyl or C₁-C₄-alkyl-C₃-C₈-cycloalkyl.

In one embodiment of the invention, R^{b} is halogen and selected from fluorine, chlorine, bromine and iodine, and preferably selected from fluorine and chlorine, and in particular, R^{b} is chlorine.

In a further embodiment of the invention, R^{b is C}₁-C₄-alkyl and selected from methyl, ethyl, n-propyl, i-propyl, n-butyl, 1-methyl-propyl, 2-methyl-propyl and 1,1-dimethylethyl, and preferably selected from methyl, ethyl, n-propyl and i-propyl, and in particular, R^{b} is methyl.

In a further embodiment of the invention, R^{b} is C₁-C₄-haloalkyl and selected from C₁-haloalkyl, C₂-haloalkyl, C₃-haloalkyl and C₄-haloalkyl. More preferably, R^{b} is C₁-haloalkyl and selected from fluormethyl, difluormethyl, trifluormethyl, chlormethyl, dichlormethyl and trichlormethyl, and in particular, R^{b} is trifluormethyl.

In a further embodiment of the invention, R^{b} is C₁-C₄-alkoxy and selected from methoxy, ethoxy, n-propyloxy, i-propyloxy, n-butyloxy, 1-methyl-propyloxy, 2-methyl-propyloxy and 1,1-dimethylethyloxy, and in particular from methoxy and ethoxy.

One embodiment relates to compounds I, wherein R is hydrogen, Y is —O— and R^{a1}, R^{a2} and R^{a3} are each independently hydrogen or have one of the definitions specified for R^{a}, especially those being preferred, which compounds are of formula I.A

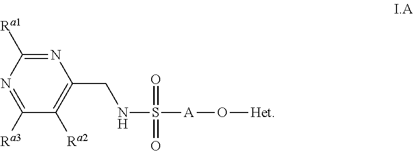

Another embodiment relates to compounds I, wherein Y is —N(R^{π})—, wherein R^{π} is hydrogen or C₁-C₄-alkyl. If R^{π} is present, in one embodiment of the invention, R^{π} is C₁-C₄-alkyl, and selected from methyl, ethyl, n-propyl, i-propyl, n-butyl, 1-methyl-propyl, 2-methyl-propyl and 1,1-dimethylethyl, and preferably selected from methyl, ethyl, n-propyl and i-propyl, and in particular, R^{π} is methyl.

A further embodiment relates to compounds I, wherein R is hydrogen, Y is —N(CH₃)— and R^{a1}, R^{a2} and R^{a3} are each independently hydrogen or have one of the definitions specified for R^{a}, especially those being preferred which compounds are of formula I.B

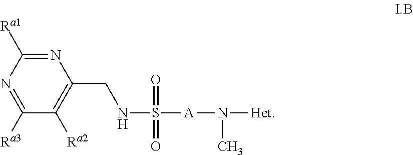

A further embodiment relates to compounds I, wherein R is hydrogen, Y is —S— and R^{a1}, R^{a2} and R^{a3} are each independently hydrogen or have one of the definitions specified for R^{a}, especially those being preferred, which compounds are of formula I.C

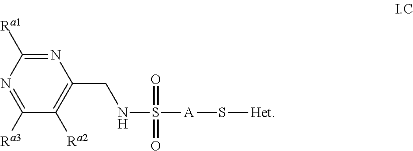

A further embodiment relates to compounds I, wherein R is hydrogen, Y is —S(═O)— and R^{a1}, R^{a2} and R^{a3} are each independently hydrogen or have one of the definitions specified for R^{a}, especially those being preferred which compounds are of formula I.D

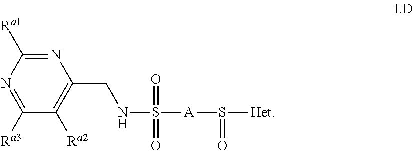

A further embodiment relates to compounds I, wherein R is hydrogen, Y is —S(═O)₂— and R^{a1}, R^{a2} and R^{a3} are each independently hydrogen or have one of the definitions specified for R^{a}, especially those being preferred, which compounds are of formula I.E

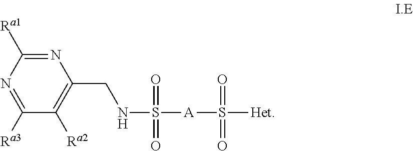

A further embodiment relates to compounds I, wherein R is hydrogen, Y is —CH₂— and R^{a1}, R^{a2} and R^{a3} are each independently hydrogen or have one of the definitions specified for R^{a}, especially those being preferred, which compounds are of formula I.F

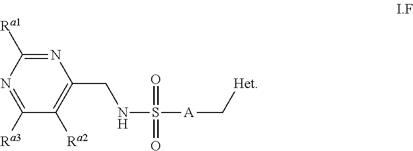

A further embodiment relates to compounds I, wherein R is hydrogen, Y is —O(CH₂)— and R^{a1}, R^{a2} and R^{a3} are each independently hydrogen or have one of the definitions specified for R^{a}, especially those being preferred, which compounds are of formula I.G

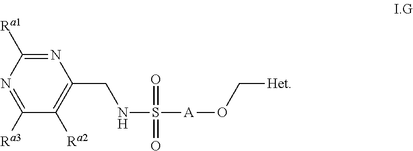

A further embodiment relates to compounds I, wherein R is hydrogen, Y is —(CH₂)O— and R^{a1}, R^{a2} and R^{a3} are each independently hydrogen or have one of the definitions specified for R^{a}, especially those being preferred, which compounds are of formula I.H

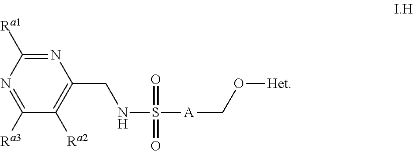

A further embodiment relates to compounds I, wherein R is hydrogen, Y is —NH— and R^{a1}, R^{a2} and R^{a3} are each independently hydrogen or have one of the definitions specified for R^{a}, especially those being preferred, which compounds are of formula I.J

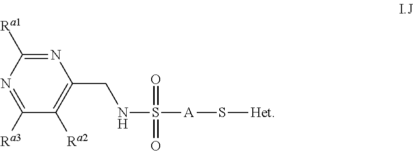

A further embodiment relates to compounds I, wherein R is hydrogen, Y is —NH— and R^{a1}, R^{a2} and R^{a3} are each independently hydrogen or have one of the definitions specified for R^{a}, especially those being preferred, which compounds are of formula I.K

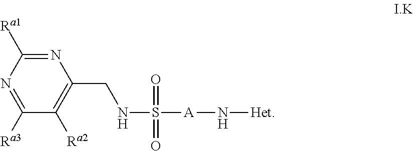

One embodiment of the invention relates to compounds I, in which Het is a 6-membered heteroaryl, wherein the ring member atoms of the heteroaryl include besides carbon atoms 1, 2, 3 or 4 heteroatoms selected from the group of N, O and S, and wherein the 6-membered heteroaryl is unsubstituted or carries 1, 2, 3 or 4 identical or different groups R^{c}.

If Het is a 6-membered heteroaryl, in one embodiment, Het carries at least one nitrogen as ring member atom. Preference is given to compounds I, in which Het is a pyridyl radical that is selected from pyridin-2-yl, pyridin-3-yl and pyridin-4-yl, and wherein the aforementioned pyridyl radicals are unsubstituted or carry 1, 2, 3 or 4 identical or different substituents Rc. More preferably, Het is pyridin-2-yl, which is unsubstituted or carries one or two radicals Rc.

Preference is given to compounds I, in which Het is a pyridazinyl radical that is selected from pyridazin-3-yl and pyridazin-4-yl, and wherein the aforementioned pyridazinyl radicals are unsubstituted or carry 1, 2 or 3 identical or different substituents R^{c}.

Preference is given to compounds I, in which Het is a pyrimidinyl radical that is selected from pyrimidin-2-yl, pyrimidin-4-yl, pyrimidin-5-yl and pyrimidin-6-yl, and wherein the aformentioned pyrimidinyl radicals are unsubstituted or carry 1, 2 or 3 identical or different substituents R^{c}.

Preference is given to compounds I, in which Het is a pyrazinyl radical that is selected from pyrazin-2-yl and pyrazin-3-yl, and wherein the aforementioned pyrazinyl radicals are unsubstituted or carry 1, 2 or 3 identical or different substituents R^{c}.

Another embodiment relates to compounds I, wherein Het is a 5-membered heteroaryl, wherein the ring member atoms of the heteroaryl include besides carbon atoms 1, 2, 3 or 4 heteroatoms selected from the group of N, O and S, and wherein the 5-membered heteroaryl is unsubstituted or carries 1, 2, 3 or 4 identical or different groups R^{c}.

If Het is a 5-membered heteroaryl, in one embodiment of the invention, Het carries one heteroatom as ring member atom. Preference is given to compounds I, in which Het is a furanyl radical that is selected from furan-2-yl and furan-3-yl, and wherein the aforementioned furanyl radicals are unsubstituted or carry 1, 2 or 3 identical or different substituents R^{c}. Preference is given to compounds I, in which Het is a thienyl radical that is selected from thien-2-yl and thien-3-yl, and wherein the aforementioned thienyl radicals are unsubstituted or carry 1, 2 or 3 identical or different substituents R^{c}. Preference is given to compounds I, in which Het is a pyrrolyl radical that is selected from pyrrol-2-yl and pyrrol-3-yl, and wherein the aforementioned pyrrolyl radicals are unsubstituted or carry 1, 2, 3 or 4 identical or different substituents R^{c}.

If Het is a 5-membered heteroaryl, in another embodiment of the invention, Het carries two heteroatoms as ring member atoms. Preference is given to compounds I, in which Het is a pyrazolyl radical that is selected from pyrazol-3-yl, pyrazol-4-yl and pyrazol-5-yl, and wherein the aforementioned pyrazolyl radicals are unsubstituted or carry 1, 2 or 3 identical or different substituents R^{c}. Preference is given to compounds I, in which Het is an isoxazolyl radical that is selected from isoxazol-3-yl, isoxazol-4-yl and isoxazol-5-yl, and wherein the aforementioned isoxazolyl radicals are unsubstituted or carry 1 or 2 identical or different substituents Rc. Preference is given to compounds I, in which Het is an isothiazolyl radical that is selected from isothiazol-3-yl, isothiazol-4-yl and isothiazol-5-yl, and wherein the aforementioned isothiazolyl radicals are unsubstituted or carry 1 or 2 identical or different substituents R^{c}. Preference is given to compounds I, in which Het is an imidazolyl radical that is selected from imidazol-2-yl, imidazol-4-yl and imidazol-5-yl, and wherein the aforementioned imidazolyl radicals are unsubstituted or carry 1, 2 or 3 identical or different substituents R^{c}. Preference is given to compounds I, in which Het is an oxazolyl radical that is selected from oxazol-2-yl, oxazol-4-yl and oxazol-5-yl, and wherein the aforementioned oxazolyl radicals are unsubstituted or carry 1 or 2 identical or different substituents R^{c}. Preference is given to compounds I, in which Het is a thiazolyl radical that is selected from thiazol-2-yl, thiazol-4-yl and thiazol-5-yl, and wherein the aforementioned thiazolyl radicals are unsubstituted or carry 1 or 2 identical or different substituents R^{c}.

If Het is a 5-membered heteroaryl, in another embodiment of the invention, Het carries 3 heteroatoms as ring member atoms.

Preferred embodiments of the invention relate to compounds I, in which the group Het is one of the following radicals H-1 to H-11:

| No. | Het |
|---|---|
| H-1 | 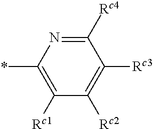 |
| H-2 | 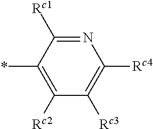 |
| H-3 | 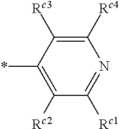 |
| H-4 | 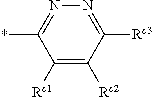 |
| H-5 | 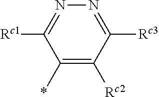 |
| H-6 | 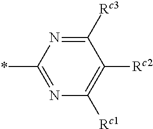 |
| H-7 | 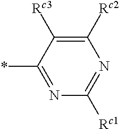 |
| H-8 | 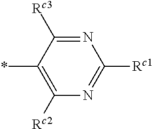 |
| H-9 | 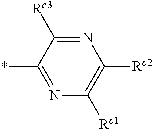 |
| H-10 | 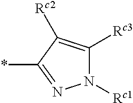 |
| H-11 | 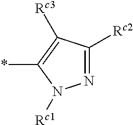 |

in which * indicates the point of attachment to Y; and R^{c1}, R^{c2}, R^{c3} and R^{c4} are each independently hydrogen or have one of the definitions specified for R^{c}, especially those being preferred.

One embodiment of the invention relates to compounds I, wherein Het carries 1, 2 or 3 radicals R^{c}. Another embodiment relates to compounds I, wherein Het carries 1 or 2 radicals R^{c}. A further embodiment relates to compounds I, wherein Het carries one radical R^{c}. A further embodiment relates to compounds I, wherein Het carries two radicals R^{c}. A further embodiment relates to compounds I, wherein Het carries 3 radicals R^{c}. A further embodiment relates to compounds I, wherein Het is unsubstituted.

In a further embodiment, two radicals R^{c} that are bound to adjacent ring member atoms of the Het group do not form together with said ring member atoms any fused cycle.

Preferably, R^{c} is halogen, CN, C₁-C₆-alkyl, C₁-C₆-haloalkyl, C₁-C₆-alkoxy, C₁-C₆-haloalkoxy, C₁-C₆-alkoxy-C₁-C₄-alkyl, C(═O)R′, C(═NOR″)R′″, C₃-C₈-cycloalkyl, C₁-C₄-alkyl-C₃-C₈-cycloalkyl, phenyl, phenoxy, phenoxy-C₁-C₄-alkyl or a 5- or 6-membered heteroaryl, wherein the ring member atoms of the heteroaryl include besides carbon atoms 1, 2, 3 or 4 heteroatoms selected from the group of N, O and S, and wherein the aforementioned cyclic radicals are unsubstituted or carry 1, 2, 3 or 4 identical or different substituents R^{d}.

In one embodiment, R^{c} is halogen and selected from fluorine, chlorine, bromine and iodine and selected from fluorine and chlorine and in particular, R^{c} is chlorine.

In another embodiment, R^{c} is CN.

In a further embodiment, R^{c} is C₁-C₄-alkyl and selected from methyl, ethyl, n-propyl, i-propyl, n-butyl, 1-methyl-propyl, 2-methyl-propyl and 1,1-dimethylethyl, and selected from methyl, ethyl, n-propyl and i-propyl, and in particular, R^{c} is methyl.

In a further embodiment, R^{c} is C₁-C₄-haloalkyl and selected from C₁-haloalkyl, C₂-haloalkyl, C₃-haloalkyl and C₄-haloalkyl. More preferably, R^{c} is C₁-haloalkyl and selected from fluormethyl, difluormethyl, trifluormethyl, chlormethyl, dichlormethyl and trichlormethyl, and in particular, R^{c} is trifluormethyl.

In a further embodiment, R^{c} is C₁-C₄-alkoxy and selected from methoxy, ethoxy, n-propyloxy, i-propyloxy, n-butyloxy, 1-methyl-propyloxy, 2-methyl-propyloxy and 1,1-dimethylethyloxy and in particular from methoxy and ethoxy.

In a further embodiment, R^{c} is C₁-C₄-haloalkoxy and specifically halomethoxy, such as difluormethoxy, trifluormethoxy, dichlormethoxy and trichlormethoxy, and haloethoxy, such as 2,2-difluorethoxy, 2,2,2-trifluorethoxy, 2,2-dichlorethoxy and 2,2,2-trichlorethoxy, and halo-n-propoxy, halo-i-propoxy, halo-n-butoxy, halo-1-methyl-propoxy, halo-2-methyl-propoxy or halo-1,1-dimethylethoxy.

In a further embodiment, R^{c} is C₃-C₈-cycloalkyl, and in particular, R^{c} is cyclopropyl.

In a further embodiment, R^{c} is phenyl.

In a further embodiment, R^{c} is phenoxy.

In a further embodiment, R^{c} is phenoxy-C₁-C₄-alkyl and selected from phenoxymethyl, 1-phenoxy-ethyl and 2-phenoxyethyl.

In a further embodiment, R^{c} is a 6-membered heteroaryl, wherein the ring member atoms of the heteroaryl include besides carbon atoms 1, 2, 3 or 4 heteroatoms selected from the group of N, O and S and is unsubstituted or carries 1, 2, 3 or 4 identical or different groups R^{d}.

If R^{c} is a 6-membered heteroaryl, in one embodiment of the invention, R^{c} carries at least one nitrogen as ring member atom. Preference is given to compounds I, in which R^{c} is a pyridyl radical that is selected from pyridin-2-yl, pyridin-3-yl and pyridin-4-yl, and wherein the aforementioned pyridyl radicals are unsubstituted or carry 1, 2, 3 or 4 identical or different substituents R^{d}.

Another embodiment relates to compounds I, wherein R^{c} is a 5-membered heteroaryl, wherein the ring member atoms of the heteroaryl include besides carbon atoms 1, 2, 3 or 4 heteroatoms selected from the group of N, O and S, and wherein R^{c} is unsubstituted or carries 1, 2, 3 or 4 identical or different groups R^{d}.

A further embodiment relates to compounds I, wherein two radicals R^{c} that are bound to adjacent ring member atoms of the Het group form together with said ring member atoms a fused cycle being a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted and carries 1, 2, 3 or 4 identical or different R^{e} radicals. In the abovementioned embodiment, the fused cycle is preferably phenyl, more preferably Het forms with said fused cycle a quinolinyl group, in particular a quinolin-4-yl group. In the abovementioned embodiment, the fused cycle is preferably a saturated carbocycle and in particular cyclohexyl. In the abovementioned embodiment, the fused cycle is preferably a partially unsaturated carbocycle and in particular cyclohexenyl.

Preference is given to compounds I, wherein two radicals R^{c} that are bound to adjacent ring member atoms of the Het group form together with said ring member atoms a fused 6-membered heteroaryl, wherein the fused 6-membered heteroaryl is unsubstituted and carries 1, 2, 3 or 4 identical or different R^{e} radicals. In the abovementioned embodiment, the fused heteroaryl is pyridyl. In the abovementioned embodiment, the fused heteroaryl is pyridazinyl. In the abovementioned embodiment, the fused heteroaryl is pyrimidinyl. In the abovementioned embodiment, the fused heteroaryl is pyrazinyl.

Preference is given to compounds I, wherein two radicals R^{c} that are bound to adjacent ring member atoms of the Het group form together with said ring member atoms a fused 5-membered heteroaryl, wherein the fused 5-membered heteroaryl is unsubstituted and carries 1, 2, 3 or 4 identical or different R^{e} radicals. In the abovementioned embodiment, the fused heteroaryl is furanyl. In the abovementioned embodiment, the fused heteroaryl is thienyl. In the abovementioned embodiment, the fused heteroaryl is pyrrolyl. In the abovementioned embodiment, the fused heteroaryl is pyrazolyl. In the abovementioned embodiment, the fused heteroaryl is isoxazolyl. In the abovementioned embodiment, the fused heteroaryl is isothiazolyl. In the abovementioned embodiment, the fused heteroaryl is imidazolyl. In the abovementioned embodiment, the fused heteroaryl is oxazolyl. In the abovementioned embodiment, the fused heteroaryl is thiazolyl.

In a specific embodiment of the invention, the two radicals R^{c} that are bound to adjacent ring member atoms of the Het group form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted.

In a further embodiment, two radicals R^{c} that are bound to adjacent ring member atoms of the Het group form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is substituted by 1, 2, 3 or 4 R^{e} radicals, and preferably, by 1, 2 or 3 R^{e} radicals, more preferably by one of two R^{e} radicals, and in particular by one radical R^{e}.

If R^{c} is present, one embodiment relates to compounds I, wherein R^{c} carries 1, 2, 3 or 4 radicals R^{d}, preferably 1, 2 or 3 radicals R^{d}, and more preferably 1 or 2 radicals R^{d}. In a particularly preferred embodiment, R^{c} carries one radical R^{d}. In another particularly preferred embodiment, R^{c} carries two radicals R^{d}. In a further particularly preferred embodiment the group R^{c} carries 3 radicals R^{d}.

In one embodiment, R^{d} is halogen and selected from fluorine, chlorine, bromine and iodine and specifically from fluorine and chlorine and in particular, R^{c} is chlorine.

In another embodiment, R^{d} is CN.

In a further embodiment, R^{d} is C₁-C₄-alkyl and selected from methyl, ethyl, n-propyl, i-propyl, n-butyl, 1-methyl-propyl, 2-methyl-propyl and 1,1-dimethylethyl, and preferably selected from methyl, ethyl, n-propyl and i-propyl and in particular, R^{d} is methyl.

In a further embodiment, R^{d} is C₁-C₄-haloalkyl and selected from C₁-haloalkyl, C₂-haloalkyl, C₃-haloalkyl and C₄-haloalkyl. More preferably, R^{c} is C₁-haloalkyl and selected from fluormethyl, difluormethyl, trifluormethyl, chlormethyl, dichlormethyl and trichlormethyl, and in particular, R^{d} is trifluormethyl.

In a further embodiment, R^{d} is C₁-C₄-alkoxy and selected from methoxy, ethoxy, n-propyloxy, i-propyloxy, n-butyloxy, 1-methyl-propyloxy, 2-methyl-propyloxy and 1,1-dimethylethyloxy and in particular from methoxy and ethoxy.

A skilled person will readily understand that the preferences given in connection with compounds of formula I also apply for formulae I.1 and I.1a and I.A to I.K as defined below.

With respect to their use, particular preference is given to the compounds I compiled in the Tables 1 to 72 below, wherein the definitions for the substituents R^{a} of the pyridine group are selected from P-1 to P-4 in Table P and wherein the definitions for group A are selected from A-1 to A-18 as described above and wherein the definitions for group Het are selected from H-1 to H-3 as described above. Here, the groups mentioned in the Tables for a substituent are furthermore, independently of the combination in which they are mentioned, a particularly preferred embodiment of the substituent in question.
Table 1: Compounds of formula I.A, wherein R^{a1}, R^{a2} and R^{a3} are defined as in line P-1 of table P, A is A-1 as defined before and the meaning of Het for each individual compound corresponds in each case to one line of table A.
Table 2: Compounds of formula I.A, wherein R^{a1}, R^{a2} and R^{a3} are defined as in line P-2 of table P, A is A-1 as defined before and the meaning of Het for each individual compound corresponds in each case to one line of table A.
Table 3: Compounds of formula I.A, wherein R^{a1}, R^{a2} and R^{a3} are defined as in line P-3 of table P, A is A-1 as defined before and the meaning of Het for each individual compound corresponds in each case to one line of table A.
Table 4: Compounds of formula I.A, wherein R^{a1}, R^{a2} and R^{a3} are defined as in line P-4 of table P, A is A-1 as defined before and the meaning of Het for each individual compound corresponds in each case to one line of table A.
Tables 5 to 8: Compounds of formula I.A, wherein R^{a1}, R^{a2} and R^{a3} are defined as in Tables 1 to 4, A is A-2 instead of A-1 and the meaning of Het for each individual compound corresponds in each case to one line of table A.
Tables 9 to 12: Compounds of formula I.A, wherein R^{a1}, R^{a2} and R^{a3} are defined as in Tables 1 to 4, A is A-3 instead of A-1 and the meaning of Het for each individual compound corresponds in each case to one line of table A.
Tables 13 to 16: Compounds of formula I.A, wherein R^{a1}, R^{a2} and R^{a3} are defined as in Tables 1 to 4, A is A-4 instead of A-1 and the meaning of Het for each individual compound corresponds in each case to one line of table A.
Tables 17 to 20: Compounds of formula I.A, wherein R^{a1}, R^{a2} and R^{a3} are defined as in Tables 1 to 4, A is A-5 instead of A-1 and the meaning of Het for each individual compound corresponds in each case to one line of table A.
Tables 21 to 24: Compounds of formula I.A, wherein R^{a1}, R^{a2} and R^{a3} are defined as in Tables 1 to 4, A is A-6 instead of A-1 and the meaning of Het for each individual compound corresponds in each case to one line of table A.
Tables 25 to 28: Compounds of formula I.A, wherein R^{a1}, R^{a2} and R^{a3} are defined as in Tables 1 to 4, A is A-7 instead of A-1 and the meaning of Het for each individual compound corresponds in each case to one line of table A.
Tables 29 to 32: Compounds of formula I.A, wherein R^{a1}, R^{a2} and R^{a3} are defined as in Tables 1 to 4, A is A-8 instead of A-1 and the meaning of Het for each individual compound corresponds in each case to one line of table A.
Tables 33 to 36: Compounds of formula I.A, wherein R^{a1}, R^{a2} and R^{a3} are defined as in Tables 1 to 4, A is A-9 instead of A-1 and the meaning of Het for each individual compound corresponds in each case to one line of table A.
Tables 37 to 40: Compounds of formula I.A, wherein R^{a1}, R^{a2} and R^{a3} are defined as in Tables 1 to 4, A is A-10 instead of A-1 and the meaning of Het for each individual compound corresponds in each case to one line of table A.
Tables 41 to 44: Compounds of formula I.A, wherein R^{a1}, R^{a2} and R^{a3} are defined as in Tables 1 to 4, A is A-11 instead of A-1 and the meaning of Het for each individual compound corresponds in each case to one line of table A.
Tables 45 to 48: Compounds of formula I.A, wherein R^{a1}, R^{a2} and R^{a3} are defined as in Tables 1 to 4, A is A-12 instead of A-1 and the meaning of Het for each individual compound corresponds in each case to one line of table A.
Tables 49 to 52: Compounds of formula I.A, wherein R^{a1}, R^{a2} and R^{a3} are defined as in Tables 1 to 4, A is A-13 instead of A-1 and the meaning of Het for each individual compound corresponds in each case to one line of table A.
Tables 53 to 56: Compounds of formula I.A, wherein R^{a1}, R^{a2} and R^{a3} are defined as in Tables 1 to 4, A is A-14 instead of A-1 and the meaning of Het for each individual compound corresponds in each case to one line of table A.
Tables 57 to 60: Compounds of formula I.A, wherein R^{a1}, R^{a2} and R^{a3} are defined as in Tables 1 to 4, A is A-15 instead of A-1 and the meaning of Het for each individual compound corresponds in each case to one line of table A.
Tables 61 to 64: Compounds of formula I.A, wherein R^{a1}, R^{a2} and R^{a3} are defined as in Tables 1 to 4, A is A-16 instead of A-1 and the meaning of Het for each individual compound corresponds in each case to one line of table A.
Tables 65 to 68: Compounds of formula I.A, wherein R^{a1}, R^{a2} and R^{a3} are defined as in Tables 1 to 4, A is A-17 instead of A-1 and the meaning of Het for each individual compound corresponds in each case to one line of table A.
Tables 69 to 72: Compounds of formula I.A, wherein R^{a1}, R^{a2} and R^{a3} are defined as in Tables 1 to 4, A is A-18 instead of A-1 and the meaning of Het for each individual compound corresponds in each case to one line of table A.

TABLE A

| line | Het | R^{aa} | R^{ab} | R^{ac} | R^{ad} |
|---|---|---|---|---|---|
| 1 | H-1 | H | H | H | H |
| 2 | H-1 | F | H | H | H |
| 3 | H-1 | Cl | H | H | H |
| 4 | H-1 | Br | H | H | H |
| 5 | H-1 | CH₃ | H | H | H |
| 6 | H-1 | CF₃ | H | H | H |
| 7 | H-1 | CHF₂ | H | H | H |
| 8 | H-1 | OCH₃ | H | H | H |
| 9 | H-1 | OCF₃ | H | H | H |
| 10 | H-1 | OCHF₂ | H | H | H |
| 11 | H-1 | SCH₃ | H | H | H |
| 12 | H-1 | H | F | H | H |
| 13 | H-1 | H | Cl | H | H |
| 14 | H-1 | H | Br | H | H |
| 15 | H-1 | H | CH₃ | H | H |
| 16 | H-1 | H | CF₃ | H | H |
| 17 | H-1 | H | CHF₂ | H | H |
| 18 | H-1 | H | OCH₃ | H | H |
| 19 | H-1 | H | OCF₃ | H | H |
| 20 | H-1 | H | OCHF₂ | H | H |
| 21 | H-1 | H | SCH₃ | H | H |
| 22 | H-1 | H | H | F | H |
| 23 | H-1 | H | H | Cl | H |
| 24 | H-1 | H | H | Br | H |
| 25 | H-1 | H | H | CH₃ | H |
| 26 | H-1 | H | H | CF₃ | H |
| 27 | H-1 | H | H | CHF₂ | H |
| 28 | H-1 | H | H | OCH₃ | H |
| 29 | H-1 | H | H | OCF₃ | H |
| 30 | H-1 | H | H | OCHF₂ | H |
| 31 | H-1 | H | H | SCH₃ | H |
| 32 | H-1 | H | H | H | F |
| 33 | H-1 | H | H | H | Cl |
| 34 | H-1 | H | H | H | Br |
| 35 | H-1 | H | H | H | CH₃ |
| 36 | H-1 | H | H | H | CF₃ |
| 37 | H-1 | H | H | H | CHF₂ |
| 38 | H-1 | H | H | H | OCH₃ |
| 39 | H-1 | H | H | H | OCF₃ |
| 40 | H-1 | H | H | H | OCHF₂ |
| 41 | H-1 | H | H | H | SCH₃ |
| 42 | H-1 | F | F | H | H |
| 43 | H-1 | Cl | F | H | H |
| 44 | H-1 | Br | F | H | H |
| 45 | H-1 | CH₃ | F | H | H |
| 46 | H-1 | CF₃ | F | H | H |
| 47 | H-1 | CHF₂ | F | H | H |
| 48 | H-1 | OCH₃ | F | H | H |
| 49 | H-1 | OCF₃ | F | H | H |
| 50 | H-1 | OCHF₂ | F | H | H |
| 51 | H-1 | SCH₃ | F | H | H |
| 52 | H-1 | F | Cl | H | H |
| 53 | H-1 | Cl | Cl | H | H |
| 54 | H-1 | Br | Cl | H | H |
| 55 | H-1 | CH₃ | Cl | H | H |
| 56 | H-1 | CF₃ | Cl | H | H |
| 57 | H-1 | CHF₂ | Cl | H | H |
| 58 | H-1 | OCH₃ | Cl | H | H |
| 59 | H-1 | OCF₃ | Cl | H | H |
| 60 | H-1 | OCHF₂ | Cl | H | H |
| 61 | H-1 | SCH₃ | Cl | H | H |
| 62 | H-1 | F | Br | H | H |
| 63 | H-1 | Cl | Br | H | H |
| 64 | H-1 | Br | Br | H | H |
| 65 | H-1 | CH₃ | Br | H | H |
| 66 | H-1 | CF₃ | Br | H | H |
| 67 | H-1 | CHF₂ | Br | H | H |
| 68 | H-1 | OCH₃ | Br | H | H |
| 69 | H-1 | OCF₃ | Br | H | H |
| 70 | H-1 | OCHF₂ | Br | H | H |
| 71 | H-1 | SCH₃ | Br | H | H |
| 72 | H-1 | F | CH₃ | H | H |
| 73 | H-1 | Cl | CH₃ | H | H |
| 74 | H-1 | Br | CH₃ | H | H |
| 75 | H-1 | CH₃ | CH₃ | H | H |
| 76 | H-1 | CF₃ | CH₃ | H | H |
| 77 | H-1 | CHF₂ | CH₃ | H | H |
| 78 | H-1 | OCH₃ | CH₃ | H | H |
| 79 | H-1 | OCF₃ | CH₃ | H | H |
| 80 | H-1 | OCHF₂ | CH₃ | H | H |
| 81 | H-1 | SCH₃ | CH₃ | H | H |
| 82 | H-1 | F | CF₃ | H | H |
| 83 | H-1 | Cl | CF₃ | H | H |
| 84 | H-1 | Br | CF₃ | H | H |
| 85 | H-1 | CH₃ | CF₃ | H | H |
| 86 | H-1 | CF₃ | CF₃ | H | H |
| 87 | H-1 | CHF₂ | CF₃ | H | H |
| 88 | H-1 | OCH₃ | CF₃ | H | H |
| 89 | H-1 | OCF₃ | CF₃ | H | H |
| 90 | H-1 | OCHF₂ | CF₃ | H | H |
| 91 | H-1 | SCH₃ | CF₃ | H | H |
| 92 | H-1 | F | CHF₂ | H | H |
| 93 | H-1 | Cl | CHF₂ | H | H |
| 94 | H-1 | Br | CHF₂ | H | H |
| 95 | H-1 | CH₃ | CHF₂ | H | H |
| 96 | H-1 | CF₃ | CHF₂ | H | H |
| 97 | H-1 | CHF₂ | CHF₂ | H | H |
| 98 | H-1 | OCH₃ | CHF₂ | H | H |
| 99 | H-1 | OCF₃ | CHF₂ | H | H |
| 100 | H-1 | OCHF₂ | CHF₂ | H | H |
| 101 | H-1 | SCH₃ | CHF₂ | H | H |
| 102 | H-1 | F | OCH₃ | H | H |
| 103 | H-1 | Cl | OCH₃ | H | H |
| 104 | H-1 | Br | OCH₃ | H | H |
| 105 | H-1 | CH₃ | OCH₃ | H | H |
| 106 | H-1 | CF₃ | OCH₃ | H | H |
| 107 | H-1 | CHF₂ | OCH₃ | H | H |
| 108 | H-1 | OCH₃ | OCH₃ | H | H |
| 109 | H-1 | OCF₃ | OCH₃ | H | H |
| 110 | H-1 | OCHF₂ | OCH₃ | H | H |
| 111 | H-1 | SCH₃ | OCH₃ | H | H |
| 112 | H-1 | F | OCF₃ | H | H |
| 113 | H-1 | Cl | OCF₃ | H | H |
| 114 | H-1 | Br | OCF₃ | H | H |
| 115 | H-1 | CH₃ | OCF₃ | H | H |
| 116 | H-1 | CF₃ | OCF₃ | H | H |
| 117 | H-1 | CHF₂ | OCF₃ | H | H |
| 118 | H-1 | OCH₃ | OCF₃ | H | H |
| 119 | H-1 | OCF₃ | OCF₃ | H | H |
| 120 | H-1 | OCHF₂ | OCF₃ | H | H |
| 121 | H-1 | SCH₃ | OCF₃ | H | H |
| 122 | H-1 | F | OCHF₂ | H | H |
| 123 | H-1 | Cl | OCHF₂ | H | H |
| 124 | H-1 | Br | OCHF₂ | H | H |
| 125 | H-1 | CH₃ | OCHF₂ | H | H |
| 126 | H-1 | CF₃ | OCHF₂ | H | H |
| 127 | H-1 | CHF₂ | OCHF₂ | H | H |
| 128 | H-1 | OCH₃ | OCHF₂ | H | H |
| 129 | H-1 | OCF₃ | OCHF₂ | H | H |
| 130 | H-1 | OCHF₂ | OCHF₂ | H | H |
| 131 | H-1 | SCH₃ | OCHF₂ | H | H |
| 132 | H-1 | F | SCH₃ | H | H |
| 133 | H-1 | Cl | SCH₃ | H | H |
| 134 | H-1 | Br | SCH₃ | H | H |
| 135 | H-1 | CH₃ | SCH₃ | H | H |
| 136 | H-1 | CF₃ | SCH₃ | H | H |
| 137 | H-1 | CHF₂ | SCH₃ | H | H |
| 138 | H-1 | OCH₃ | SCH₃ | H | H |
| 139 | H-1 | OCHF₂ | SCH₃ | H | H |
| 140 | H-1 | OCF₃ | SCH₃ | H | H |
| 141 | H-1 | SCH₃ | SCH₃ | H | H |
| 142 | H-1 | F | H | F | H |
| 143 | H-1 | Cl | H | F | H |
| 144 | H-1 | Br | H | F | H |
| 145 | H-1 | CH₃ | H | F | H |
| 146 | H-1 | CF₃ | H | F | H |
| 147 | H-1 | CHF₂ | H | F | H |
| 148 | H-1 | OCH₃ | H | F | H |
| 149 | H-1 | OCF₃ | H | F | H |
| 150 | H-1 | OCHF₂ | H | F | H |
| 151 | H-1 | SCH₃ | H | F | H |
| 152 | H-1 | F | H | Cl | H |
| 153 | H-1 | Cl | H | Cl | H |
| 154 | H-1 | Br | H | Cl | H |
| 155 | H-1 | CH₃ | H | Cl | H |
| 156 | H-1 | CF₃ | H | Cl | H |
| 157 | H-1 | CHF₂ | H | Cl | H |
| 158 | H-1 | OCH₃ | H | Cl | H |
| 159 | H-1 | OCF₃ | H | Cl | H |
| 160 | H-1 | OCHF₂ | H | Cl | H |
| 161 | H-1 | SCH₃ | H | Cl | H |
| 162 | H-1 | F | H | Br | H |
| 163 | H-1 | Cl | H | Br | H |
| 164 | H-1 | Br | H | Br | H |
| 165 | H-1 | CH₃ | H | Br | H |
| 166 | H-1 | CF₃ | H | Br | H |
| 167 | H-1 | CHF₂ | H | Br | H |
| 168 | H-1 | OCH₃ | H | Br | H |
| 169 | H-1 | OCF₃ | H | Br | H |
| 170 | H-1 | OCHF₂ | H | Br | H |
| 171 | H-1 | SCH₃ | H | Br | H |
| 172 | H-1 | F | H | CH₃ | H |
| 173 | H-1 | Cl | H | CH₃ | H |
| 174 | H-1 | Br | H | CH₃ | H |
| 175 | H-1 | CH₃ | H | CH₃ | H |
| 176 | H-1 | CF₃ | H | CH₃ | H |
| 177 | H-1 | CHF₂ | H | CH₃ | H |
| 178 | H-1 | OCH₃ | H | CH₃ | H |
| 179 | H-1 | OCF₃ | H | CH₃ | H |
| 180 | H-1 | OCHF₂ | H | CH₃ | H |
| 181 | H-1 | SCH₃ | H | CH₃ | H |
| 182 | H-1 | F | H | CF₃ | H |
| 183 | H-1 | Cl | H | CF₃ | H |
| 184 | H-1 | Br | H | CF₃ | H |
| 185 | H-1 | CH₃ | H | CF₃ | H |
| 186 | H-1 | CF₃ | H | CF₃ | H |
| 187 | H-1 | CHF₂ | H | CF₃ | H |
| 188 | H-1 | OCH₃ | H | CF₃ | H |
| 189 | H-1 | OCF₃ | H | CF₃ | H |
| 190 | H-1 | OCHF₂ | H | CF₃ | H |
| 191 | H-1 | SCH₃ | H | CF₃ | H |
| 192 | H-1 | F | H | CHF₂ | H |
| 193 | H-1 | Cl | H | CHF₂ | H |
| 194 | H-1 | Br | H | CHF₂ | H |
| 195 | H-1 | CH₃ | H | CHF₂ | H |
| 196 | H-1 | CF₃ | H | CHF₂ | H |
| 197 | H-1 | CHF₂ | H | CHF₂ | H |
| 198 | H-1 | OCH₃ | H | CHF₂ | H |
| 199 | H-1 | OCF₃ | H | CHF₂ | H |
| 200 | H-1 | OCHF₂ | H | CHF₂ | H |
| 201 | H-1 | SCH₃ | H | CHF₂ | H |
| 202 | H-1 | F | H | OCH₃ | H |
| 203 | H-1 | Cl | H | OCH₃ | H |
| 204 | H-1 | Br | H | OCH₃ | H |
| 205 | H-1 | CH₃ | H | OCH₃ | H |
| 206 | H-1 | CF₃ | H | OCH₃ | H |
| 207 | H-1 | CHF₂ | H | OCH₃ | H |
| 208 | H-1 | OCH₃ | H | OCH₃ | H |
| 209 | H-1 | OCF₃ | H | OCH₃ | H |
| 210 | H-1 | OCHF₂ | H | OCH₃ | H |
| 211 | H-1 | SCH₃ | H | OCH₃ | H |
| 212 | H-1 | F | H | OCF₃ | H |
| 213 | H-1 | Cl | H | OCF₃ | H |
| 214 | H-1 | Br | H | OCF₃ | H |
| 215 | H-1 | CH₃ | H | OCF₃ | H |
| 216 | H-1 | CF₃ | H | OCF₃ | H |
| 217 | H-1 | CHF₂ | H | OCF₃ | H |
| 218 | H-1 | OCH₃ | H | OCF₃ | H |
| 219 | H-1 | OCF₃ | H | OCF₃ | H |
| 220 | H-1 | OCHF₂ | H | OCF₃ | H |
| 221 | H-1 | SCH₃ | H | OCF₃ | H |
| 222 | H-1 | F | H | OCHF₂ | H |
| 223 | H-1 | Cl | H | OCHF₂ | H |
| 224 | H-1 | Br | H | OCHF₂ | H |
| 225 | H-1 | CH₃ | H | OCHF₂ | H |
| 226 | H-1 | CF₃ | H | OCHF₂ | H |
| 227 | H-1 | CHF₂ | H | OCHF₂ | H |
| 228 | H-1 | OCH₃ | H | OCHF₂ | H |
| 229 | H-1 | OCF₃ | H | OCHF₂ | H |
| 230 | H-1 | OCHF₂ | H | OCHF₂ | H |
| 231 | H-1 | SCH₃ | H | OCHF₂ | H |
| 232 | H-1 | F | H | SCH₃ | H |
| 233 | H-1 | Cl | H | SCH₃ | H |
| 234 | H-1 | Br | H | SCH₃ | H |
| 235 | H-1 | CH₃ | H | SCH₃ | H |
| 236 | H-1 | CF₃ | H | SCH₃ | H |
| 237 | H-1 | CHF₂ | H | SCH₃ | H |
| 238 | H-1 | OCH₃ | H | SCH₃ | H |
| 239 | H-1 | OCF₃ | H | SCH₃ | H |
| 240 | H-1 | OCHF₂ | H | SCH₃ | H |
| 241 | H-1 | SCH₃ | H | SCH₃ | H |
| 242 | H-1 | F | H | H | F |
| 243 | H-1 | Cl | H | H | F |
| 244 | H-1 | Br | H | H | F |
| 245 | H-1 | CH₃ | H | H | F |
| 246 | H-1 | CF₃ | H | H | F |
| 247 | H-1 | CHF₂ | H | H | F |
| 248 | H-1 | OCH₃ | H | H | F |
| 249 | H-1 | OCF₃ | H | H | F |
| 250 | H-1 | OCHF₂ | H | H | F |
| 251 | H-1 | SCH₃ | H | H | F |
| 252 | H-1 | F | H | H | Cl |
| 253 | H-1 | Cl | H | H | Cl |
| 254 | H-1 | Br | H | H | Cl |
| 255 | H-1 | CH₃ | H | H | Cl |
| 256 | H-1 | CF₃ | H | H | Cl |
| 257 | H-1 | CHF₂ | H | H | Cl |
| 258 | H-1 | OCH₃ | H | H | Cl |
| 259 | H-1 | OCF₃ | H | H | Cl |
| 260 | H-1 | OCHF₂ | H | H | Cl |
| 261 | H-1 | SCH₃ | H | H | Cl |
| 262 | H-1 | F | H | H | Br |
| 263 | H-1 | Cl | H | H | Br |
| 264 | H-1 | Br | H | H | Br |
| 265 | H-1 | CH₃ | H | H | Br |
| 266 | H-1 | CF₃ | H | H | Br |
| 267 | H-1 | CHF₂ | H | H | Br |
| 268 | H-1 | OCH₃ | H | H | Br |
| 269 | H-1 | OCF₃ | H | H | Br |
| 270 | H-1 | OCHF₂ | H | H | Br |
| 271 | H-1 | SCH₃ | H | H | Br |
| 272 | H-1 | F | H | H | CH₃ |
| 273 | H-1 | Cl | H | H | CH₃ |
| 274 | H-1 | Br | H | H | CH₃ |
| 275 | H-1 | CH₃ | H | H | CH₃ |
| 276 | H-1 | CF₃ | H | H | CH₃ |
| 277 | H-1 | CHF₂ | H | H | CH₃ |
| 278 | H-1 | OCH₃ | H | H | CH₃ |
| 279 | H-1 | OCF₃ | H | H | CH₃ |
| 280 | H-1 | OCHF₂ | H | H | CH₃ |
| 281 | H-1 | SCH₃ | H | H | CH₃ |
| 282 | H-1 | F | H | H | CF₃ |
| 283 | H-1 | Cl | H | H | CF₃ |
| 284 | H-1 | Br | H | H | CF₃ |
| 285 | H-1 | CH₃ | H | H | CF₃ |
| 286 | H-1 | CF₃ | H | H | CF₃ |
| 287 | H-1 | CHF₂ | H | H | CF₃ |
| 288 | H-1 | OCH₃ | H | H | CF₃ |
| 289 | H-1 | OCF₃ | H | H | CF₃ |
| 290 | H-1 | OCHF₂ | H | H | CF₃ |
| 291 | H-1 | SCH₃ | H | H | CF₃ |
| 292 | H-1 | F | H | H | CHF₂ |
| 293 | H-1 | Cl | H | H | CHF₂ |
| 294 | H-1 | Br | H | H | CHF₂ |
| 295 | H-1 | CH₃ | H | H | CHF₂ |
| 296 | H-1 | CF₃ | H | H | CHF₂ |
| 297 | H-1 | CHF₂ | H | H | CHF₂ |
| 298 | H-1 | OCH₃ | H | H | CHF₂ |
| 299 | H-1 | OCF₃ | H | H | CHF₂ |
| 300 | H-1 | OCHF₂ | H | H | CHF₂ |
| 301 | H-1 | SCH₃ | H | H | CHF₂ |
| 302 | H-1 | F | H | H | OCH₃ |
| 303 | H-1 | Cl | H | H | OCH₃ |
| 304 | H-1 | Br | H | H | OCH₃ |
| 305 | H-1 | CH₃ | H | H | OCH₃ |
| 306 | H-1 | CF₃ | H | H | OCH₃ |
| 307 | H-1 | CHF₂ | H | H | OCH₃ |
| 308 | H-1 | OCH₃ | H | H | OCH₃ |
| 309 | H-1 | OCF₃ | H | H | OCH₃ |
| 310 | H-1 | OCHF₂ | H | H | OCH₃ |
| 311 | H-1 | SCH₃ | H | H | OCH₃ |
| 312 | H-1 | F | H | H | OCF₃ |
| 313 | H-1 | Cl | H | H | OCF₃ |
| 314 | H-1 | Br | H | H | OCF₃ |
| 315 | H-1 | CH₃ | H | H | OCF₃ |
| 316 | H-1 | CF₃ | H | H | OCF₃ |
| 317 | H-1 | CHF₂ | H | H | OCF₃ |
| 318 | H-1 | OCH₃ | H | H | OCF₃ |
| 319 | H-1 | OCF₃ | H | H | OCF₃ |
| 320 | H-1 | OCHF₂ | H | H | OCF₃ |
| 321 | H-1 | SCH₃ | H | H | OCF₃ |
| 322 | H-1 | F | H | H | OCHF₂ |
| 323 | H-1 | Cl | H | H | OCHF₂ |
| 324 | H-1 | Br | H | H | OCHF₂ |
| 325 | H-1 | CH₃ | H | H | OCHF₂ |
| 326 | H-1 | CF₃ | H | H | OCHF₂ |
| 327 | H-1 | CHF₂ | H | H | OCHF₂ |
| 328 | H-1 | OCH₃ | H | H | OCHF₂ |
| 329 | H-1 | OCF₃ | H | H | OCHF₂ |
| 330 | H-1 | OCHF₂ | H | H | OCHF₂ |
| 331 | H-1 | SCH₃ | H | H | OCHF₂ |
| 332 | H-1 | F | H | H | SCH₃ |
| 333 | H-1 | Cl | H | H | SCH₃ |
| 334 | H-1 | Br | H | H | SCH₃ |
| 335 | H-1 | CH₃ | H | H | SCH₃ |
| 336 | H-1 | CF₃ | H | H | SCH₃ |
| 337 | H-1 | CHF₂ | H | H | SCH₃ |
| 338 | H-1 | OCH₃ | H | H | SCH₃ |
| 339 | H-1 | OCF₃ | H | H | SCH₃ |
| 340 | H-1 | OCHF₂ | H | H | SCH₃ |
| 341 | H-1 | SCH₃ | H | H | SCH₃ |
| 342 | H-1 | H | F | F | H |
| 343 | H-1 | H | Cl | F | H |
| 344 | H-1 | H | Br | F | H |
| 345 | H-1 | H | CH₃ | F | H |
| 346 | H-1 | H | CF₃ | F | H |
| 347 | H-1 | H | CHF₂ | F | H |
| 348 | H-1 | H | OCH₃ | F | H |
| 349 | H-1 | H | OCF₃ | F | H |
| 350 | H-1 | H | OCHF₂ | F | H |
| 351 | H-1 | H | SCH₃ | F | H |
| 352 | H-1 | H | F | Cl | H |
| 353 | H-1 | H | Cl | Cl | H |
| 354 | H-1 | H | Br | Cl | H |
| 355 | H-1 | H | CH₃ | Cl | H |
| 356 | H-1 | H | CF₃ | Cl | H |
| 357 | H-1 | H | CHF₂ | Cl | H |
| 358 | H-1 | H | OCH₃ | Cl | H |
| 359 | H-1 | H | OCF₃ | Cl | H |
| 360 | H-1 | H | OCHF₂ | Cl | H |
| 361 | H-1 | H | SCH₃ | Cl | H |
| 362 | H-1 | H | F | Br | H |
| 363 | H-1 | H | Cl | Br | H |
| 364 | H-1 | H | Br | Br | H |
| 365 | H-1 | H | CH₃ | Br | H |
| 366 | H-1 | H | CF₃ | Br | H |
| 367 | H-1 | H | CHF₂ | Br | H |
| 368 | H-1 | H | OCH₃ | Br | H |
| 369 | H-1 | H | OCF₃ | Br | H |
| 370 | H-1 | H | OCHF₂ | Br | H |
| 371 | H-1 | H | SCH₃ | Br | H |
| 372 | H-1 | H | F | CH₃ | H |
| 373 | H-1 | H | Cl | CH₃ | H |
| 374 | H-1 | H | Br | CH₃ | H |
| 375 | H-1 | H | CH₃ | CH₃ | H |
| 376 | H-1 | H | CF₃ | CH₃ | H |
| 377 | H-1 | H | CHF₂ | CH₃ | H |
| 378 | H-1 | H | OCH₃ | CH₃ | H |
| 379 | H-1 | H | OCF₃ | CH₃ | H |
| 380 | H-1 | H | OCHF₂ | CH₃ | H |
| 381 | H-1 | H | SCH₃ | CH₃ | H |
| 382 | H-1 | H | F | CF₃ | H |
| 383 | H-1 | H | Cl | CF₃ | H |
| 384 | H-1 | H | Br | CF₃ | H |
| 385 | H-1 | H | CH₃ | CF₃ | H |
| 386 | H-1 | H | CF₃ | CF₃ | H |
| 387 | H-1 | H | CHF₂ | CF₃ | H |
| 388 | H-1 | H | OCH₃ | CF₃ | H |
| 389 | H-1 | H | OCF₃ | CF₃ | H |
| 390 | H-1 | H | OCHF₂ | CF₃ | H |
| 391 | H-1 | H | SCH₃ | CF₃ | H |
| 392 | H-1 | H | F | CHF₂ | H |
| 393 | H-1 | H | Cl | CHF₂ | H |
| 394 | H-1 | H | Br | CHF₂ | H |
| 395 | H-1 | H | CH₃ | CHF₂ | H |
| 396 | H-1 | H | CF₃ | CHF₂ | H |
| 397 | H-1 | H | CHF₂ | CHF₂ | H |
| 398 | H-1 | H | OCH₃ | CHF₂ | H |
| 399 | H-1 | H | OCF₃ | CHF₂ | H |
| 400 | H-1 | H | OCHF₂ | CHF₂ | H |
| 401 | H-1 | H | SCH₃ | CHF₂ | H |
| 402 | H-1 | H | F | OCH₃ | H |
| 403 | H-1 | H | Cl | OCH₃ | H |
| 404 | H-1 | H | Br | OCH₃ | H |
| 405 | H-1 | H | CH₃ | OCH₃ | H |
| 406 | H-1 | H | CF₃ | OCH₃ | H |
| 407 | H-1 | H | CHF₂ | OCH₃ | H |
| 408 | H-1 | H | OCH₃ | OCH₃ | H |
| 409 | H-1 | H | OCF₃ | OCH₃ | H |
| 410 | H-1 | H | OCHF₂ | OCH₃ | H |
| 411 | H-1 | H | SCH₃ | OCH₃ | H |
| 412 | H-1 | H | F | OCF₃ | H |
| 413 | H-1 | H | Cl | OCF₃ | H |
| 414 | H-1 | H | Br | OCF₃ | H |
| 415 | H-1 | H | CH₃ | OCF₃ | H |
| 416 | H-1 | H | CF₃ | OCF₃ | H |
| 417 | H-1 | H | CHF₂ | OCF₃ | H |
| 418 | H-1 | H | OCH₃ | OCF₃ | H |
| 419 | H-1 | H | OCF₃ | OCF₃ | H |
| 420 | H-1 | H | OCHF₂ | OCF₃ | H |
| 421 | H-1 | H | SCH₃ | OCF₃ | H |
| 422 | H-1 | H | F | OCHF₂ | H |
| 423 | H-1 | H | Cl | OCHF₂ | H |
| 424 | H-1 | H | Br | OCHF₂ | H |
| 425 | H-1 | H | CH₃ | OCHF₂ | H |
| 426 | H-1 | H | CF₃ | OCHF₂ | H |
| 427 | H-1 | H | CHF₂ | OCHF₂ | H |
| 428 | H-1 | H | OCH₃ | OCHF₂ | H |
| 429 | H-1 | H | OCF₃ | OCHF₂ | H |
| 430 | H-1 | H | OCHF₂ | OCHF₂ | H |
| 431 | H-1 | H | SCH₃ | OCHF₂ | H |
| 432 | H-1 | H | F | SCH₃ | H |
| 433 | H-1 | H | Cl | SCH₃ | H |
| 434 | H-1 | H | Br | SCH₃ | H |
| 435 | H-1 | H | CH₃ | SCH₃ | H |
| 436 | H-1 | H | CF₃ | SCH₃ | H |
| 437 | H-1 | H | CHF₂ | SCH₃ | H |
| 438 | H-1 | H | OCH₃ | SCH₃ | H |
| 439 | H-1 | H | OCF₃ | SCH₃ | H |
| 440 | H-1 | H | OCHF₂ | SCH₃ | H |
| 441 | H-1 | H | SCH₃ | SCH₃ | H |
| 442 | H-1 | H | F | H | F |
| 443 | H-1 | H | Cl | H | F |
| 444 | H-1 | H | Br | H | F |
| 445 | H-1 | H | CH₃ | H | F |
| 446 | H-1 | H | CF₃ | H | F |
| 447 | H-1 | H | CHF₂ | H | F |
| 448 | H-1 | H | OCH₃ | H | F |
| 449 | H-1 | H | OCF₃ | H | F |
| 450 | H-1 | H | OCHF₂ | H | F |
| 451 | H-1 | H | SCH₃ | H | F |
| 452 | H-1 | H | F | H | Cl |
| 453 | H-1 | H | Cl | H | Cl |
| 454 | H-1 | H | Br | H | Cl |
| 455 | H-1 | H | CH₃ | H | Cl |
| 456 | H-1 | H | CF₃ | H | Cl |
| 457 | H-1 | H | CHF₂ | H | Cl |
| 458 | H-1 | H | OCH₃ | H | Cl |
| 459 | H-1 | H | OCF₃ | H | Cl |
| 460 | H-1 | H | OCHF₂ | H | Cl |
| 461 | H-1 | H | SCH₃ | H | Cl |
| 462 | H-1 | H | F | H | Br |
| 463 | H-1 | H | Cl | H | Br |
| 464 | H-1 | H | Br | H | Br |
| 465 | H-1 | H | CH₃ | H | Br |
| 466 | H-1 | H | CF₃ | H | Br |
| 467 | H-1 | H | CHF₂ | H | Br |
| 468 | H-1 | H | OCH₃ | H | Br |
| 469 | H-1 | H | OCF₃ | H | Br |
| 470 | H-1 | H | OCHF₂ | H | Br |
| 471 | H-1 | H | SCH₃ | H | Br |
| 472 | H-1 | H | F | H | CH₃ |
| 473 | H-1 | H | Cl | H | CH₃ |
| 474 | H-1 | H | Br | H | CH₃ |
| 475 | H-1 | H | CH₃ | H | CH₃ |
| 476 | H-1 | H | CF₃ | H | CH₃ |
| 477 | H-1 | H | CHF₂ | H | CH₃ |
| 478 | H-1 | H | OCH₃ | H | CH₃ |
| 479 | H-1 | H | OC₂H₅ | H | CH₃ |
| 480 | H-1 | H | OCF₃ | H | CH₃ |
| 481 | H-1 | H | SCH₃ | H | CH₃ |
| 482 | H-1 | H | F | H | CF₃ |
| 483 | H-1 | H | Cl | H | CF₃ |
| 484 | H-1 | H | Br | H | CF₃ |
| 485 | H-1 | H | CH₃ | H | CF₃ |
| 486 | H-1 | H | CF₃ | H | CF₃ |
| 487 | H-1 | H | CHF₂ | H | CF₃ |
| 488 | H-1 | H | OCH₃ | H | CF₃ |
| 489 | H-1 | H | OC₂H₅ | H | CF₃ |
| 490 | H-1 | H | OCF₃ | H | CF₃ |
| 491 | H-1 | H | SCH₃ | H | CF₃ |
| 492 | H-1 | H | F | H | CHF₂ |
| 493 | H-1 | H | Cl | H | CHF₂ |
| 494 | H-1 | H | Br | H | CHF₂ |
| 495 | H-1 | H | CH₃ | H | CHF₂ |
| 496 | H-1 | H | CF₃ | H | CHF₂ |
| 497 | H-1 | H | CHF₂ | H | CHF₂ |
| 498 | H-1 | H | OCH₃ | H | CHF₂ |
| 499 | H-1 | H | OCHF₂ | H | CHF₂ |
| 500 | H-1 | H | OCF₃ | H | CHF₂ |
| 501 | H-1 | H | SCH₃ | H | CHF₂ |
| 502 | H-1 | H | F | H | OCH₃ |
| 503 | H-1 | H | Cl | H | OCH₃ |
| 504 | H-1 | H | Br | H | OCH₃ |
| 505 | H-1 | H | CH₃ | H | OCH₃ |
| 506 | H-1 | H | CF₃ | H | OCH₃ |
| 507 | H-1 | H | CHF₂ | H | OCH₃ |
| 508 | H-1 | H | OCH₃ | H | OCH₃ |
| 509 | H-1 | H | OCF₃ | H | OCH₃ |
| 510 | H-1 | H | OCHF₂ | H | OCH₃ |
| 511 | H-1 | H | SCH₃ | H | OCH₃ |
| 512 | H-1 | H | F | H | OCF₃ |
| 513 | H-1 | H | Cl | H | OCF₃ |
| 514 | H-1 | H | Br | H | OCF₃ |
| 515 | H-1 | H | CH₃ | H | OCF₃ |
| 516 | H-1 | H | CF₃ | H | OCF₃ |
| 517 | H-1 | H | CHF₂ | H | OCF₃ |
| 518 | H-1 | H | OCH₃ | H | OCF₃ |
| 519 | H-1 | H | OCF₃ | H | OCF₃ |
| 520 | H-1 | H | OCHF₂ | H | OCF₃ |
| 521 | H-1 | H | SCH₃ | H | OCF₃ |
| 522 | H-1 | H | F | H | OCHF₂ |
| 523 | H-1 | H | Cl | H | OCHF₂ |
| 524 | H-1 | H | Br | H | OCHF₂ |
| 525 | H-1 | H | CH₃ | H | OCHF₂ |
| 526 | H-1 | H | CF₃ | H | OCHF₂ |
| 527 | H-1 | H | CHF₂ | H | OCHF₂ |
| 528 | H-1 | H | OCH₃ | H | OCHF₂ |
| 529 | H-1 | H | OCF₃ | H | OCHF₂ |
| 530 | H-1 | H | OCHF₂ | H | OCHF₂ |
| 531 | H-1 | H | SCH₃ | H | OCHF₂ |
| 532 | H-1 | H | F | H | SCH₃ |
| 533 | H-1 | H | Cl | H | SCH₃ |
| 534 | H-1 | H | Br | H | SCH₃ |
| 535 | H-1 | H | CH₃ | H | SCH₃ |
| 536 | H-1 | H | CF₃ | H | SCH₃ |
| 537 | H-1 | H | CHF₂ | H | SCH₃ |
| 538 | H-1 | H | OCH₃ | H | SCH₃ |
| 539 | H-1 | H | OCF₃ | H | SCH₃ |
| 540 | H-1 | H | OCHF₂ | H | SCH₃ |
| 541 | H-1 | H | SCH₃ | H | SCH₃ |
| 542 | H-1 | H | H | F | F |
| 543 | H-1 | H | H | Cl | F |
| 544 | H-1 | H | H | Br | F |
| 545 | H-1 | H | H | CH₃ | F |
| 546 | H-1 | H | H | CF₃ | F |
| 547 | H-1 | H | H | CHF₂ | F |
| 548 | H-1 | H | H | OCH₃ | F |
| 549 | H-1 | H | H | OCF₃ | F |
| 550 | H-1 | H | H | OCHF₂ | F |
| 551 | H-1 | H | H | SCH₃ | F |
| 552 | H-1 | H | H | F | Cl |
| 553 | H-1 | H | H | Cl | Cl |
| 554 | H-1 | H | H | Br | Cl |
| 555 | H-1 | H | H | CH₃ | Cl |
| 556 | H-1 | H | H | CF₃ | Cl |
| 557 | H-1 | H | H | CHF₂ | Cl |
| 558 | H-1 | H | H | OCH₃ | Cl |
| 559 | H-1 | H | H | OCF₃ | Cl |
| 560 | H-1 | H | H | OCHF₂ | Cl |
| 561 | H-1 | H | H | SCH₃ | Cl |
| 562 | H-1 | H | H | F | Br |
| 563 | H-1 | H | H | Cl | Br |
| 564 | H-1 | H | H | Br | Br |
| 565 | H-1 | H | H | CH₃ | Br |
| 566 | H-1 | H | H | CF₃ | Br |
| 567 | H-1 | H | H | CHF₂ | Br |
| 568 | H-1 | H | H | OCH₃ | Br |
| 569 | H-1 | H | H | OCF₃ | Br |
| 570 | H-1 | H | H | OCHF₂ | Br |
| 571 | H-1 | H | H | SCH₃ | Br |
| 572 | H-1 | H | H | F | CH₃ |
| 573 | H-1 | H | H | Cl | CH₃ |
| 574 | H-1 | H | H | Br | CH₃ |
| 575 | H-1 | H | H | CH₃ | CH₃ |
| 576 | H-1 | H | H | CF₃ | CH₃ |
| 577 | H-1 | H | H | CHF₂ | CH₃ |
| 578 | H-1 | H | H | OCH₃ | CH₃ |
| 579 | H-1 | H | H | OCF₃ | CH₃ |
| 580 | H-1 | H | H | OCHF₂ | CH₃ |
| 581 | H-1 | H | H | SCH₃ | CH₃ |
| 582 | H-1 | H | H | F | CF₃ |
| 583 | H-1 | H | H | Cl | CF₃ |
| 584 | H-1 | H | H | Br | CF₃ |
| 585 | H-1 | H | H | CH₃ | CF₃ |
| 586 | H-1 | H | H | CF₃ | CF₃ |
| 587 | H-1 | H | H | CHF₂ | CF₃ |
| 588 | H-1 | H | H | OCH₃ | CF₃ |
| 589 | H-1 | H | H | OCF₃ | CF₃ |
| 590 | H-1 | H | H | OCHF₂ | CF₃ |
| 591 | H-1 | H | H | SCH₃ | CF₃ |
| 592 | H-1 | H | H | F | CHF₂ |
| 593 | H-1 | H | H | Cl | CHF₂ |
| 594 | H-1 | H | H | Br | CHF₂ |
| 595 | H-1 | H | H | CH₃ | CHF₂ |
| 596 | H-1 | H | H | CF₃ | CHF₂ |
| 597 | H-1 | H | H | CHF₂ | CHF₂ |
| 598 | H-1 | H | H | OCH₃ | CHF₂ |
| 599 | H-1 | H | H | OCF₃ | CHF₂ |
| 600 | H-1 | H | H | OCHF₂ | CHF₂ |
| 601 | H-1 | H | H | SCH₃ | CHF₂ |
| 602 | H-1 | H | H | F | OCH₃ |
| 603 | H-1 | H | H | Cl | OCH₃ |
| 604 | H-1 | H | H | Br | OCH₃ |
| 605 | H-1 | H | H | CH₃ | OCH₃ |
| 606 | H-1 | H | H | CF₃ | OCH₃ |
| 607 | H-1 | H | H | CHF₂ | OCH₃ |
| 608 | H-1 | H | H | OCH₃ | OCH₃ |
| 609 | H-1 | H | H | OCF₃ | OCH₃ |
| 610 | H-1 | H | H | OCHF₂ | OCH₃ |
| 611 | H-1 | H | H | SCH₃ | OCH₃ |
| 612 | H-1 | H | H | F | OCF₃ |
| 613 | H-1 | H | H | Cl | OCF₃ |
| 614 | H-1 | H | H | Br | OCF₃ |
| 615 | H-1 | H | H | CH₃ | OCF₃ |
| 616 | H-1 | H | H | CF₃ | OCF₃ |
| 617 | H-1 | H | H | CHF₂ | OCF₃ |
| 618 | H-1 | H | H | OCH₃ | OCF₃ |
| 619 | H-1 | H | H | OCF₃ | OCF₃ |
| 620 | H-1 | H | H | OCHF₂ | OCF₃ |
| 621 | H-1 | H | H | SCH₃ | OCF₃ |
| 622 | H-3 | H | H | H | H |
| 623 | H-3 | F | H | H | H |
| 624 | H-3 | Cl | H | H | H |
| 625 | H-3 | Br | H | H | H |
| 626 | H-3 | CH₃ | H | H | H |
| 627 | H-3 | CF₃ | H | H | H |
| 628 | H-3 | CHF₂ | H | H | H |
| 629 | H-3 | OCH₃ | H | H | H |
| 630 | H-3 | OCF₃ | H | H | H |
| 631 | H-3 | OCHF₂ | H | H | H |
| 632 | H-3 | SCH₃ | H | H | H |
| 633 | H-3 | H | F | H | H |
| 634 | H-3 | H | Cl | H | H |
| 635 | H-3 | H | Br | H | H |
| 636 | H-3 | H | CH₃ | H | H |
| 637 | H-3 | H | CF₃ | H | H |
| 638 | H-3 | H | CHF₂ | H | H |
| 639 | H-3 | H | OCH₃ | H | H |
| 640 | H-3 | H | OCF₃ | H | H |
| 641 | H-3 | H | OCHF₂ | H | H |
| 642 | H-3 | H | SCH₃ | H | H |
| 643 | H-3 | F | F | H | H |
| 644 | H-3 | Cl | F | H | H |
| 645 | H-3 | Br | F | H | H |
| 646 | H-3 | CH₃ | F | H | H |
| 647 | H-3 | CF₃ | F | H | H |
| 648 | H-3 | CHF₂ | F | H | H |
| 649 | H-3 | OCH₃ | F | H | H |
| 650 | H-3 | OCF₃ | F | H | H |
| 651 | H-3 | OCHF₂ | F | H | H |
| 652 | H-3 | SCH₃ | F | H | H |
| 653 | H-3 | F | Cl | H | H |
| 654 | H-3 | Cl | Cl | H | H |
| 655 | H-3 | Br | Cl | H | H |
| 656 | H-3 | CH₃ | Cl | H | H |
| 657 | H-3 | CF₃ | Cl | H | H |
| 658 | H-3 | CHF₂ | Cl | H | H |
| 659 | H-3 | OCH₃ | Cl | H | H |
| 660 | H-3 | OCF₃ | Cl | H | H |
| 661 | H-3 | OCHF₂ | Cl | H | H |
| 662 | H-3 | SCH₃ | Cl | H | H |
| 663 | H-3 | F | Br | H | H |
| 664 | H-3 | Cl | Br | H | H |
| 665 | H-3 | Br | Br | H | H |
| 666 | H-3 | CH₃ | Br | H | H |
| 667 | H-3 | CF₃ | Br | H | H |
| 668 | H-3 | CHF₂ | Br | H | H |
| 669 | H-3 | OCH₃ | Br | H | H |
| 670 | H-3 | OCF₃ | Br | H | H |
| 671 | H-3 | OCHF₂ | Br | H | H |
| 672 | H-3 | SCH₃ | Br | H | H |
| 673 | H-3 | F | CH₃ | H | H |
| 674 | H-3 | Cl | CH₃ | H | H |
| 675 | H-3 | Br | CH₃ | H | H |
| 676 | H-3 | CH₃ | CH₃ | H | H |
| 677 | H-3 | CF₃ | CH₃ | H | H |
| 678 | H-3 | CHF₂ | CH₃ | H | H |
| 679 | H-3 | OCH₃ | CH₃ | H | H |
| 680 | H-3 | OCF₃ | CH₃ | H | H |
| 681 | H-3 | OCHF₂ | CH₃ | H | H |
| 682 | H-3 | SCH₃ | CH₃ | H | H |
| 683 | H-3 | F | CF₃ | H | H |
| 684 | H-3 | Cl | CF₃ | H | H |
| 685 | H-3 | Br | CF₃ | H | H |
| 686 | H-3 | CH₃ | CF₃ | H | H |
| 687 | H-3 | CF₃ | CF₃ | H | H |
| 688 | H-3 | CHF₂ | CF₃ | H | H |
| 689 | H-3 | OCH₃ | CF₃ | H | H |
| 690 | H-3 | OCF₃ | CF₃ | H | H |
| 691 | H-3 | OCHF₂ | CF₃ | H | H |
| 692 | H-3 | SCH₃ | CF₃ | H | H |
| 693 | H-3 | F | CHF₂ | H | H |
| 694 | H-3 | Cl | CHF₂ | H | H |
| 695 | H-3 | Br | CHF₂ | H | H |
| 696 | H-3 | CH₃ | CHF₂ | H | H |
| 697 | H-3 | CF₃ | CHF₂ | H | H |
| 698 | H-3 | CHF₂ | CHF₂ | H | H |
| 699 | H-3 | OCH₃ | CHF₂ | H | H |
| 700 | H-3 | OCF₃ | CHF₂ | H | H |
| 701 | H-3 | OCHF₂ | CHF₂ | H | H |
| 702 | H-3 | SCH₃ | CHF₂ | H | H |
| 703 | H-3 | F | OCH₃ | H | H |
| 704 | H-3 | Cl | OCH₃ | H | H |
| 705 | H-3 | Br | OCH₃ | H | H |
| 706 | H-3 | CH₃ | OCH₃ | H | H |
| 707 | H-3 | CF₃ | OCH₃ | H | H |
| 708 | H-3 | CHF₂ | OCH₃ | H | H |
| 709 | H-3 | OCH₃ | OCH₃ | H | H |
| 710 | H-3 | OCF₃ | OCH₃ | H | H |
| 711 | H-3 | OCHF₂ | OCH₃ | H | H |
| 712 | H-3 | SCH₃ | OCH₃ | H | H |
| 713 | H-3 | F | OCF₃ | H | H |
| 714 | H-3 | Cl | OCF₃ | H | H |
| 715 | H-3 | Br | OCF₃ | H | H |
| 716 | H-3 | CH₃ | OCF₃ | H | H |
| 717 | H-3 | CF₃ | OCF₃ | H | H |
| 718 | H-3 | CHF₂ | OCF₃ | H | H |
| 719 | H-3 | OCH₃ | OCF₃ | H | H |
| 720 | H-3 | OCF₃ | OCF₃ | H | H |
| 721 | H-3 | OCHF₂ | OCF₃ | H | H |
| 722 | H-3 | SCH₃ | OCF₃ | H | H |
| 723 | H-3 | F | OCHF₂ | H | H |
| 724 | H-3 | Cl | OCHF₂ | H | H |
| 725 | H-3 | Br | OCHF₂ | H | H |
| 726 | H-3 | CH₃ | OCHF₂ | H | H |
| 727 | H-3 | CF₃ | OCHF₂ | H | H |
| 728 | H-3 | CHF₂ | OCHF₂ | H | H |
| 729 | H-3 | OCH₃ | OCHF₂ | H | H |
| 730 | H-3 | OCF₃ | OCHF₂ | H | H |
| 731 | H-3 | OCHF₂ | OCHF₂ | H | H |
| 732 | H-3 | SCH₃ | OCHF₂ | H | H |
| 733 | H-3 | F | SCH₃ | H | H |
| 734 | H-3 | Cl | SCH₃ | H | H |
| 735 | H-3 | Br | SCH₃ | H | H |
| 736 | H-3 | CH₃ | SCH₃ | H | H |
| 737 | H-3 | CF₃ | SCH₃ | H | H |
| 738 | H-3 | CHF₂ | SCH₃ | H | H |
| 739 | H-3 | OCH₃ | SCH₃ | H | H |
| 740 | H-3 | OCF₃ | SCH₃ | H | H |
| 741 | H-3 | OCHF₂ | SCH₃ | H | H |
| 742 | H-3 | SCH₃ | SCH₃ | H | H |
| 743 | H-3 | F | H | F | H |
| 744 | H-3 | Cl | H | F | H |
| 745 | H-3 | Br | H | F | H |
| 746 | H-3 | CH₃ | H | F | H |
| 747 | H-3 | CF₃ | H | F | H |
| 748 | H-3 | CHF₂ | H | F | H |
| 749 | H-3 | OCH₃ | H | F | H |
| 750 | H-3 | OCF₃ | H | F | H |
| 751 | H-3 | OCHF₂ | H | F | H |
| 752 | H-3 | SCH₃ | H | F | H |
| 753 | H-3 | F | H | Cl | H |
| 754 | H-3 | Cl | H | Cl | H |
| 755 | H-3 | Br | H | Cl | H |
| 756 | H-3 | CH₃ | H | Cl | H |
| 757 | H-3 | CF₃ | H | Cl | H |
| 758 | H-3 | CHF₂ | H | Cl | H |
| 759 | H-3 | OCH₃ | H | Cl | H |
| 760 | H-3 | OCF₃ | H | Cl | H |
| 761 | H-3 | OCHF₂ | H | Cl | H |
| 762 | H-3 | SCH₃ | H | Cl | H |
| 763 | H-3 | F | H | Br | H |
| 764 | H-3 | Cl | H | Br | H |
| 765 | H-3 | Br | H | Br | H |
| 766 | H-3 | CH₃ | H | Br | H |
| 767 | H-3 | CF₃ | H | Br | H |
| 768 | H-3 | CHF₂ | H | Br | H |
| 769 | H-3 | OCH₃ | H | Br | H |
| 770 | H-3 | OCF₃ | H | Br | H |
| 771 | H-3 | OCHF₂ | H | Br | H |
| 772 | H-3 | SCH₃ | H | Br | H |
| 773 | H-3 | F | H | CH₃ | H |
| 774 | H-3 | Cl | H | CH₃ | H |
| 775 | H-3 | Br | H | CH₃ | H |
| 776 | H-3 | CH₃ | H | CH₃ | H |
| 777 | H-3 | CF₃ | H | CH₃ | H |
| 778 | H-3 | CHF₂ | H | CH₃ | H |
| 779 | H-3 | OCH₃ | H | CH₃ | H |
| 780 | H-3 | OCF₃ | H | CH₃ | H |
| 781 | H-3 | OCHF₂ | H | CH₃ | H |
| 782 | H-3 | SCH₃ | H | CH₃ | H |
| 783 | H-3 | F | H | CF₃ | H |
| 784 | H-3 | Cl | H | CF₃ | H |
| 785 | H-3 | Br | H | CF₃ | H |
| 786 | H-3 | CH₃ | H | CF₃ | H |
| 787 | H-3 | CF₃ | H | CF₃ | H |
| 788 | H-3 | CHF₂ | H | CF₃ | H |
| 789 | H-3 | OCH₃ | H | CF₃ | H |
| 790 | H-3 | OCF₃ | H | CF₃ | H |
| 791 | H-3 | OCHF₂ | H | CF₃ | H |
| 792 | H-3 | SCH₃ | H | CF₃ | H |
| 793 | H-3 | F | H | CHF₂ | H |
| 794 | H-3 | Cl | H | CHF₂ | H |
| 795 | H-3 | Br | H | CHF₂ | H |
| 796 | H-3 | CH₃ | H | CHF₂ | H |
| 797 | H-3 | CF₃ | H | CHF₂ | H |
| 798 | H-3 | CHF₂ | H | CHF₂ | H |
| 799 | H-3 | OCH₃ | H | CHF₂ | H |
| 800 | H-3 | OCF₃ | H | CHF₂ | H |
| 801 | H-3 | OCHF₂ | H | CHF₂ | H |
| 802 | H-3 | SCH₃ | H | CHF₂ | H |
| 803 | H-3 | F | H | OCH₃ | H |
| 804 | H-3 | Cl | H | OCH₃ | H |
| 805 | H-3 | Br | H | OCH₃ | H |
| 806 | H-3 | CH₃ | H | OCH₃ | H |
| 807 | H-3 | CF₃ | H | OCH₃ | H |
| 808 | H-3 | CHF₂ | H | OCH₃ | H |
| 809 | H-3 | OCH₃ | H | OCH₃ | H |
| 810 | H-3 | OCF₃ | H | OCH₃ | H |
| 811 | H-3 | OCHF₂ | H | OCH₃ | H |
| 812 | H-3 | SCH₃ | H | OCH₃ | H |
| 813 | H-3 | F | H | OCF₃ | H |
| 814 | H-3 | Cl | H | OCF₃ | H |
| 815 | H-3 | Br | H | OCF₃ | H |
| 816 | H-3 | CH₃ | H | OCF₃ | H |
| 817 | H-3 | CF₃ | H | OCF₃ | H |
| 818 | H-3 | CHF₂ | H | OCF₃ | H |
| 819 | H-3 | OCH₃ | H | OCF₃ | H |
| 820 | H-3 | OCF₃ | H | OCF₃ | H |
| 821 | H-3 | OCHF₂ | H | OCF₃ | H |
| 822 | H-3 | SCH₃ | H | OCF₃ | H |
| 823 | H-3 | F | H | OCHF₂ | H |
| 824 | H-3 | Cl | H | OCHF₂ | H |
| 825 | H-3 | Br | H | OCHF₂ | H |
| 826 | H-3 | CH₃ | H | OCHF₂ | H |
| 827 | H-3 | CF₃ | H | OCHF₂ | H |
| 828 | H-3 | CHF₂ | H | OCHF₂ | H |
| 829 | H-3 | OCH₃ | H | OCHF₂ | H |
| 830 | H-3 | OCF₃ | H | OCHF₂ | H |
| 831 | H-3 | OCHF₂ | H | OCHF₂ | H |
| 832 | H-3 | SCH₃ | H | OCHF₂ | H |
| 833 | H-3 | F | H | SCH₃ | H |
| 834 | H-3 | Cl | H | SCH₃ | H |
| 835 | H-3 | Br | H | SCH₃ | H |
| 836 | H-3 | CH₃ | H | SCH₃ | H |
| 837 | H-3 | CF₃ | H | SCH₃ | H |
| 838 | H-3 | CHF₂ | H | SCH₃ | H |
| 839 | H-3 | OCH₃ | H | SCH₃ | H |
| 840 | H-3 | OCF₃ | H | SCH₃ | H |
| 841 | H-3 | OCHF₂ | H | SCH₃ | H |
| 842 | H-3 | SCH₃ | H | SCH₃ | H |
| 843 | H-3 | F | H | H | F |
| 844 | H-3 | Cl | H | H | F |
| 845 | H-3 | Br | H | H | F |
| 846 | H-3 | CH₃ | H | H | F |
| 847 | H-3 | CF₃ | H | H | F |
| 848 | H-3 | CHF₂ | H | H | F |
| 849 | H-3 | OCH₃ | H | H | F |
| 850 | H-3 | OCF₃ | H | H | F |
| 851 | H-3 | OCHF₂ | H | H | F |
| 852 | H-3 | SCH₃ | H | H | F |
| 853 | H-3 | Cl | H | H | Cl |
| 854 | H-3 | Br | H | H | Cl |
| 855 | H-3 | CH₃ | H | H | Cl |
| 856 | H-3 | CF₃ | H | H | Cl |
| 857 | H-3 | CHF₂ | H | H | Cl |
| 858 | H-3 | OCH₃ | H | H | Cl |
| 859 | H-3 | OCF₃ | H | H | Cl |
| 860 | H-3 | OCHF₂ | H | H | Cl |
| 861 | H-3 | SCH₃ | H | H | Cl |
| 862 | H-3 | Br | H | H | Br |
| 863 | H-3 | CH₃ | H | H | Br |
| 864 | H-3 | CF₃ | H | H | Br |
| 865 | H-3 | CHF₂ | H | H | Br |
| 866 | H-3 | OCH₃ | H | H | Br |
| 867 | H-3 | OCF₃ | H | H | Br |
| 868 | H-3 | OCHF₂ | H | H | Br |
| 869 | H-3 | SCH₃ | H | H | Br |
| 870 | H-3 | CH₃ | H | H | CH₃ |
| 871 | H-3 | CF₃ | H | H | CH₃ |
| 872 | H-3 | CHF₂ | H | H | CH₃ |
| 873 | H-3 | OCH₃ | H | H | CH₃ |
| 874 | H-3 | OCF₃ | H | H | CH₃ |
| 875 | H-3 | OCHF₂ | H | H | CH₃ |
| 876 | H-3 | SCH₃ | H | H | CH₃ |
| 877 | H-3 | CF₃ | H | H | CF₃ |
| 878 | H-3 | CHF₂ | H | H | CF₃ |
| 879 | H-3 | OCH₃ | H | H | CF₃ |
| 880 | H-3 | OCHF₂ | H | H | CF₃ |
| 881 | H-3 | SCH₃ | H | H | CF₃ |
| 882 | H-3 | CHF₂ | H | H | CHF₂ |
| 883 | H-3 | OCH₃ | H | H | CHF₂ |
| 884 | H-3 | OCF₃ | H | H | CHF₂ |
| 885 | H-3 | OCHF₂ | H | H | CHF₂ |
| 886 | H-3 | SCH₃ | H | H | CHF₂ |
| 887 | H-3 | OCH₃ | H | H | OCH₃ |
| 888 | H-3 | OCF₃ | H | H | OCH₃ |
| 889 | H-3 | OCHF₂ | H | H | OCH₃ |
| 890 | H-3 | SCH₃ | H | H | OCH₃ |
| 891 | H-3 | OCF₃ | H | H | OCF₃ |
| 892 | H-3 | OCHF₂ | H | H | OCF₃ |
| 893 | H-3 | SCH₃ | H | H | OCF₃ |
| 894 | H-3 | OCHF₂ | H | H | OCHF₂ |
| 895 | H-3 | SCH₃ | H | H | OCHF₂ |
| 896 | H-3 | SCH₃ | H | H | SCH₃ |
| 897 | H-3 | H | F | F | H |
| 898 | H-3 | H | Cl | F | H |
| 899 | H-3 | H | Br | F | H |
| 900 | H-3 | H | CH₃ | F | H |
| 901 | H-3 | H | CF₃ | F | H |
| 902 | H-3 | H | CHF₂ | F | H |
| 903 | H-3 | H | OCH₃ | F | H |
| 904 | H-3 | H | OCF₃ | F | H |
| 905 | H-3 | H | OCHF₂ | F | H |
| 906 | H-3 | H | SCH₃ | F | H |
| 907 | H-3 | H | Cl | Cl | H |
| 908 | H-3 | H | Br | Cl | H |
| 909 | H-3 | H | CH₃ | Cl | H |
| 910 | H-3 | H | CF₃ | Cl | H |
| 911 | H-3 | H | CHF₂ | Cl | H |
| 912 | H-3 | H | OCH₃ | Cl | H |
| 913 | H-3 | H | OCF₃ | Cl | H |
| 914 | H-3 | H | OCHF₂ | Cl | H |
| 915 | H-3 | H | SCH₃ | Cl | H |
| 916 | H-3 | H | Br | Br | H |
| 917 | H-3 | H | CH₃ | Br | H |
| 918 | H-3 | H | CF₃ | Br | H |
| 919 | H-3 | H | CHF₂ | Br | H |
| 920 | H-3 | H | OCH₃ | Br | H |
| 921 | H-3 | H | OCF₃ | Br | H |
| 922 | H-3 | H | OCHF₂ | Br | H |
| 923 | H-3 | H | SCH₃ | Br | H |
| 924 | H-3 | H | CH₃ | CH₃ | H |
| 925 | H-3 | H | CF₃ | CH₃ | H |
| 926 | H-3 | H | CHF₂ | CH₃ | H |
| 927 | H-3 | H | OCH₃ | CH₃ | H |
| 928 | H-3 | H | OCF₃ | CH₃ | H |
| 929 | H-3 | H | OCHF₂ | CH₃ | H |
| 930 | H-3 | H | SCH₃ | CH₃ | H |
| 931 | H-3 | H | CF₃ | CF₃ | H |
| 932 | H-3 | H | CHF₂ | CF₃ | H |
| 933 | H-3 | H | OCH₃ | CF₃ | H |
| 934 | H-3 | H | OCF₃ | CF₃ | H |
| 935 | H-3 | H | OCHF₂ | CF₃ | H |
| 936 | H-3 | H | SCH₃ | CF₃ | H |
| 937 | H-3 | H | CHF₂ | CHF₂ | H |
| 938 | H-3 | H | OCH₃ | CHF₂ | H |
| 939 | H-3 | H | OCF₃ | CHF₂ | H |
| 940 | H-3 | H | OCHF₂ | CHF₂ | H |
| 941 | H-3 | H | SCH₃ | CHF₂ | H |
| 942 | H-3 | H | OCH₃ | OCH₃ | H |
| 943 | H-3 | H | OCF₃ | OCH₃ | H |
| 944 | H-3 | H | OCHF₂ | OCH₃ | H |
| 945 | H-3 | H | SCH₃ | OCH₃ | H |
| 946 | H-3 | H | OCF₃ | OCF₃ | H |
| 947 | H-3 | H | OCHF₂ | OCF₃ | H |
| 948 | H-3 | H | SCH₃ | OCF₃ | H |
| 949 | H-3 | H | OCHF₂ | OCHF₂ | H |
| 950 | H-3 | H | SCH₃ | OCHF₂ | H |
| 951 | H-3 | H | SCH₃ | SCH₃ | H |

The inventive compounds I can be prepared by various routes in analogy to prior art processes known per se for preparing sulfonamide compounds and, advantageously, by the synthesis shown in the following schemes and in the experimental part of this application.

A pyrimidin-4-ylmethylamine compound II can be reacted with a compound Ill to obtain a compound I according to the present invention as shown below, wherein n, R, R^{a}, Y and Het are as defined above, and L is a leaving group such as halogen, optionally substituted phenoxy, optionally substituted heteroaryloxy, N₃, or heteroaryl, preferably pentafluorphenoxy, hydroxybenzotriazolyloxy, heteroaryl such as imazolyl, pyrazolyl or triazolyl, and halogen such as chloro, fluoro or bromo:

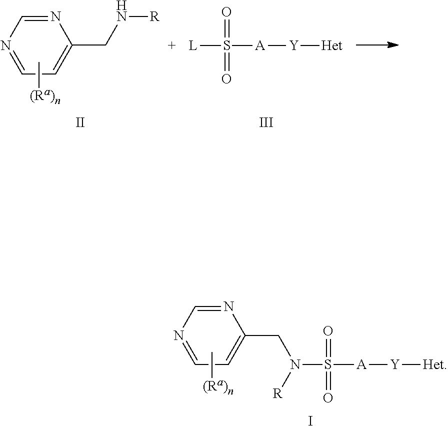

The reaction of compound III with compound II can be performed in accordance with standard methods of organic chemistry, see for example, Liebigs Ann. Chem. 641, 1990, or WO 05/033081. The reaction of sulfonic acid phenyl ester derivatives of compound III with compound II can be performed in accordance with methods described in Bioorg. Med. Chem. Lett. 17(14), 3972-3977, 2007; Chem. Commun. (10), 1074-1076, 2007; or Tetrahedron Lett. 46(44), 7637-7640, 2005.

This reaction is usually carried out in an inert organic solvent. Suitable solvents are aliphatic hydrocarbons, aromatic hydrocarbons, such as toluene, o-, m- and p-xylene, halogenated hydrocarbons, such as dichloromethane (DCM), chloroform and chlorobenzene, ethers, such as diethyl ether, diisopropyl ether, methyl tert.-butyl ether (MTBE), dioxane, anisole and tetrahydrofuran (THF), nitriles, such as acetonitrile and propionitrile, ketones, such as acetone, methyl ethyl ketone, diethyl ketone and tert.-butyl methyl ketone, and also dimethyl sulfoxide (DMSO), dimethylformamide (DMF), dimethyl acetamide, N-methyl-2-pyrrolidone (NMP), N-methyl-2-pyrrolidone (NEP) and acetic acid ethyl ester, preferably dichloromethane, acetontirile, toluene, benzene, THF, dioxane, pyridine, MTBE, NMP, acetonitrile, toluene diethyl ether, acetic acid ethyl ester, DMSO or DMF. It is also possible to use mixtures of the solvents mentioned.

The reaction is carried out in the presence of a base. Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, alkali metal and alkaline earth metal phosphates, alkali metal and alkaline earth metal hydrides, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine and NMP, pyridine, substituted pyridines, such as collidine, lutidine and 4 dimethylaminopyridine, and also bicyclic amines. Particular preference is given to sodium hydroxide, potassium hydroxide, potassium carbonate, potassium bicarbonate and sodium carbonate. The bases are generally employed in equimolar amounts, in excess or, if appropriate, as solvent. The excess of base is typically 0.5 to 5 molar equivalents relative to 1 mole of compounds II.

Generally, the reaction is carried out at temperatures of from −30° C. to 120 C, preferably from −10° C. to 100° C.

The starting materials, i.e. compounds II and compounds III, are generally reacted with one another in equimolar amounts.

Accordingly, a further aspect of the present invention relates to a process for preparing compounds I as defined before, which comprises reacting an aminomethylpyrimidine compound of formula II

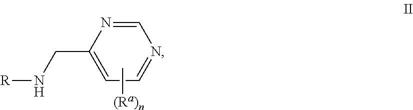

wherein n, R and R^{a} have one of the meanings given above, under basic conditions with a sulfonic acid compound of formula III

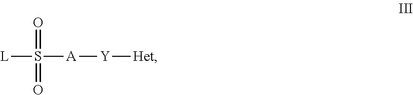

wherein A, Y and Het have one of the meanings given above and L is a leaving group selected from chloro, fluoro, azido, optionally substituted heteroaryl, optionally substituted heteroaryloxy or optionally substituted phenoxy, wherein the heteroaryl radical is selected from pyrazol-1-yl, imidazol-1-yl, 1,2,3-triazol-1-yl and 1,2,4-triazol-1-yl, and wherein the heteroaryl, heteroaryloxy and phenoxy radicals are unsubstituted or carry one, two, three, four or five identical or different substituents selected from halogen, C₁-C₄-alkyl and C₁-C₄-haloalkyl, and/or two substituents that are bound to adjacent ring member atoms of the heteroaryl, heteroaryloxy and phenoxy radicals may form together with said ring member atoms a fused 5-, 6- or 7-membered saturated, partially unsaturated or aromatic carbocycle or heterocycle, wherein the ring member atoms of the fused heterocycle include besides carbon atoms one, two, three or four heteroatoms selected from the group of N, O and S, and wherein the fused carbocycle or heterocycle is unsubstituted or carries one, two, three or four identical or different substituents selected from halogen, C₁-C₄-alkyl and C₁-C₄-haloalkyl.

Alternatively, a sulfonamide compound III.a can be reacted with a compound IV to obtain directly a compound I as shown below, wherein n, R^{a}, R, A, Y and Het are as defined above, and L is a leaving group as defined above for compounds III:

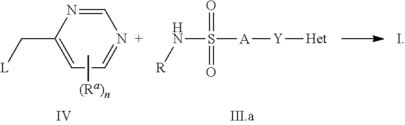

For this reaction, the conditions for reacting compounds II with compounds III may be used as described above.

Alternatively, this reaction may also be carried out in two consecutive steps as shown below, wherein n, R^{a}, R, A, Y and Het are as defined above, and L is a leaving group as defined above for compounds III:

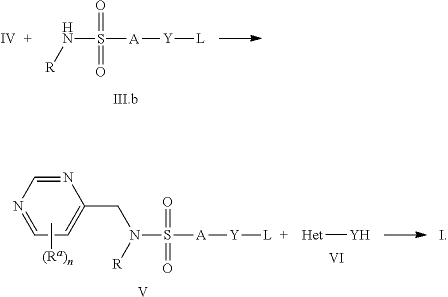

For both reactions, the conditions for reacting compounds II with compounds III may be used as described above.

Alternatively, compounds I may also be obtained by first reacting a compound VII with an aminomethylpyrimidine compound II to obtain compound VIII. This product can be reacted with a compound VI to obtain a compound I as shown below, wherein R^{a}, n, R, A, Y and Het are as defined above, and L¹ and L² are leaving groups as defined above for compounds III:

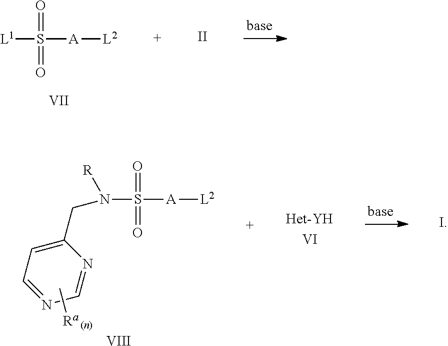

For both reactions, the conditions for reacting compounds II with compounds III may be used as described above.

Pyridimin-4-ylmethylamine compounds II are known from the literature (e.g. from WO 06/097489, WO 02/066470, U.S. Pat. No. 4,482,437 or JP 04243867) or are commercially available or they can be prepared for example by reduction of the corresponding oxime IX.a, nitrile IX.b, or amide IX.c as described below. Appropriate methods therefor are known to those skilled in the art and shown below, wherein R, R^{a} and n have one of the meanings given above:

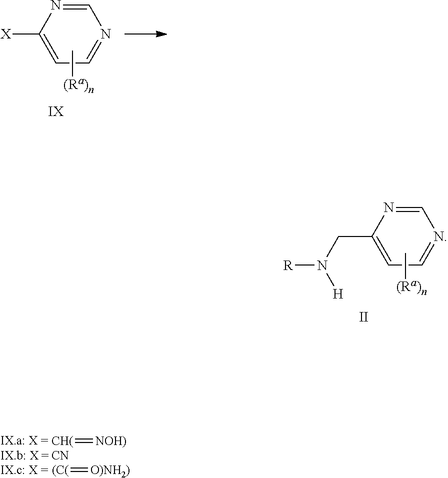

Methods suitable for the reduction of an oxime compound IX.a to the corresponding amine compound II have been described in the literature e.g. in March, J. “Advanced Organic Chemistry: Reactions, Mechanisms, and Structure” (John Wiley & Sons, New York, 4th edition, 1992, 1218-1219).

Methods suitable for the reduction of a nitrile compound IX.b to the corresponding amine compound II have been described in the literature, e.g. in March, J. “Advanced Organic Chemistry: Reactions, Mechanisms, and Structure” (John Wiley & Sons, New York, 4th edition, 1992, 918-919).

Methods suitable for the reduction of an amide compound IX.c to the corresponding amine compound II have been described in the literature, e.g. in March, J. “Advanced Organic Chemistry : Reactions, Mechanisms, and Structure” (John Wiley & Sons, New York, 4th edition, 1992, 1212-1213).

The oxime compound IX.a can be prepared for example from either the respective aldehyd compound (X═CHO; compound IX.d) or the methylderivative (X═CH₃; compound IX.e), in analogy to Houben-Weyl, Vol. 10/4, Thieme, Stuttgart, 1968; vol. 11/2, 1957; vol E5, 1985; J. Prakt. Chem-Chem. Ztg. 336(8), 695-697, 1994; Tetrahedron Lett. 42(39), 6815-6818, 2001; or Heterocycles 29(9), 1741-1760, 1989.

Oxime compounds IX.a, wherein one substuent R^{a} is 2-methoxy, are novel. Accordingly the invention relates also to intermediates IX.a

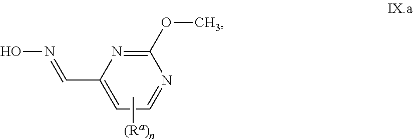

wherein R^{a} is defined as described above and n is zero, one or two.
Table IX.a: Oxime compounds of formula IX.a′

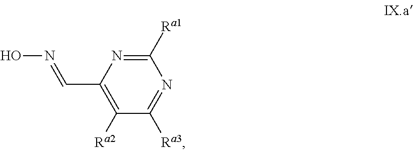

wherein R^{a1}, R^{a2} and R^{a3} are each independently hydrogen or have one of the definitions specified for R^{a} and the meaning of R^{a1}, R^{a2} and R^{a3} for each individual compound corresponds in each case to one line of table P.

The aldehyd compound IX.d can be synthesized from a compound IX.e in analogy to J. Org. Chem. 51(4), pp. 536-537, 1986, or from a haloderivative (X=halogen, compound IX.f) as shown in Eur. J. Org. Chem., 2003, (8), pp. 1576-1588; Tetrahedron Lett. 1999, 40 (19), pp. 3719-3722; Tetrahedron, 1999, 55 (41), pp. 12149-12156.

The nitrile compound IX.b is either commercially available or can be prepared in analogie to the route described in Heterocycles, 41(4), 675 (1995), Chem. Pharm. Bull., 21, 1927 (1973) or J. Chem. Soc., 426 (1942), e.g. from the corresponding halo compound IX.f by reaction with CuCN, NaCN or KCN. The compounds IX.f are either commercially available or can be synthesized according to standard methods.

The amide compound IX.c can be prepared, for example, from the corresponding carboxylic acid chloride by reaction with ammonia.

A further method to build up compounds II is shown below, wherein n and R^{a} are as defined above and Boc is tert-butyloxycarbonyl:

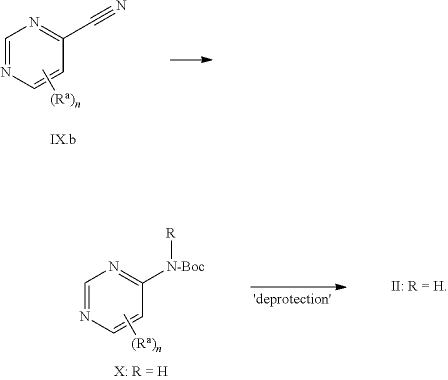

The hydrogenation of the nitrile IX.b in the presence of a catalyst, such as Raney nickel or palladium-on-carbon and t-butyl dicarbonate affords the N-protected compound X, wherein R is hydrogen. On treating with hydrogen bromide/glacial acetic acid or with trifluoroacetic acid containing water, the compound X can be deprotected to yield a compound II, wherein R is hydrogen.

Compounds X or II, wherein R is hydrogen, can be converted by conventional processes such as alkylation. Examples of suitable alkylating agents include alkyl halides, such as alkyl chloride, alkyl bromide or alkyl iodide, examples being methyl chloride, methyl bromide or methyl iodide, or dialkyl sulfates such as dimethyl sulfate or diethyl sulfate. The reaction with the alkylating agent is carried out advantageously in the presence of a solvent. Solvents used for these reactions are—depending on temperature range—aliphatic, cycloaliphatic or aromatic hydrocarbons such as hexane, cyclohexane, toluene, xylene, chlorinated aliphatic and aromatic hydrocarbons such as DCM, chlorobenzene, open-chain dialkyl ethers such as diethyl ether, di-n-propyl ether, MTBE, cyclic ethers such as THF, 1,4-dioxane, glycol ethers such as dimethyl glycol ether, or mixtures of these solvents.

Compounds II, wherein R^{a} is alkoxy, haloalkoxy, alkylthio or haloalkylthio can be prepared in analogy to standard processes from a compound X wherein R^{a} is halogen, especially chlorine, for example in analogy to methods described in J. Heterocycl. Chem. (2005), 42(7), 1369-1379; Tetrahedron Lett. 47(26), 4415-4418, 2006; or Chem. Pharm. Bull. 31(121. 4533-8. 1983. This synthesis route is shown below:

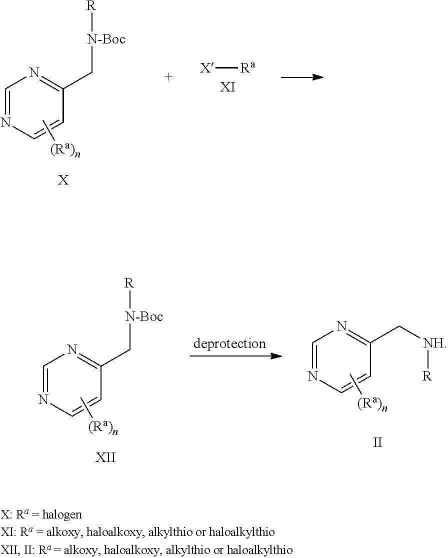

A compound X is reacted with a compound X′—R^{a} (also referred to as compound XI) to give a compound XII. Depending on the R^{a} group to be introduced, compounds XI are inorganic alkoxides, haloalkoxides, thiolates or halothiolates. The reaction is effected advantageously in an inert solvent. The cation X′ in formula XI is of little importance; for practical reasons, ammonium salts, tetraalkylammonium salts such as tetramethylammonium or tetraethylammonium salts, or alkali metal salts or alkaline earth metal salts are typically preferred. Suitable solvents comprise ethers such as dioxane, diethyl ether, MTBE and preferably THF, halogenated hydrocarbons such as DCM or dichloroethane, aromatic hydrocarbons such as toluene, and mixtures thereof. Deprotection of the amino group in formula XII to give the desired compound II can be accomplished as described above for deprotection of compounds X.

Compounds II, wherein R^{a} is alkyl, haloalkyl, alkenyl, alkynyl, cycloalkyl or alkylcycloalkyl, can advantageously be prepared by reacting compounds II, wherein R^{a} is halogen, with organometallic compounds R^{a}-Mt, wherein R^{a} is alkyl, haloalkyl, alkenyl, alkynyl, cycloalkyl or alkyl-cycloalkyl and Mt is lithium, magnesium or zinc. The reaction is effected preferably in the presence of catalytic or, in particular, at least equimolar amounts of transition metal salts and/or compounds, in particular in the presence of Cu salts such as Cu(I) halides and especially Cu(I) iodide, or Pd-catalyzed. The reaction is effected generally in an inert organic solvent, for example one of the aforementioned ethers, in particular THF, an aliphatic or cycloaliphatic hydrocarbon such as hexane, cyclohexane and the like, an aromatic hydrocarbon such as toluene, or in a mixture of these solvents. The temperatures required for this purpose are in the range of from −100 to +100° C. and especially in the range from −80 to +40° C.

A further method to build up compounds II from mucohalo acids, such as mucochloric or mucobromic acid is shown below, wherein n and R^{a} are as defined above, preferably R^{a} is C₁-C₄-alkyl, methyl, methoxy, methylthio or hydroxy, and X is bromine or chlorine:

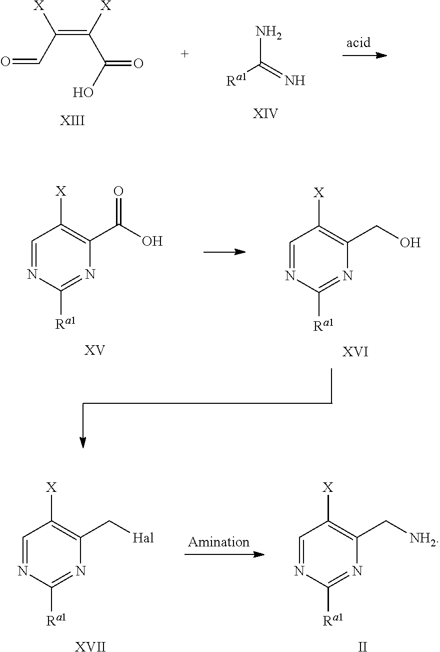

A mucohalo acid compound XIII is advanageously reacted in presence of a base to obtain a compound XV (cf. Synth. Commun. 37(13), 2231-2241, 2007). Suitable bases are, in general, inorganic compounds, such as alkali metal and alkaline earth carbonates such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine and NMP or pyridine. Particular preference is given to triethylamine, diisopropylethylamine, sodium carbonate, sodium bicarbonate or potassium bicarbonate. The next reaction step converts compounds XI to compounds XV via formation of the acid chloride followed by reduction with NaBH₄ at low temperature (cf. J. Med. Chem. 29(8), 1374-80, 1986). Via halogenation the hydroxy group of compound XVI is converted to a halogen (Hal) to obtain a compound XVII. The halogenation is advantageously effected in the presence of a solvent and of customary halogenation agents such as a sulfonyl chloride derivative in combination with a metal halide or triphenylphosphin together with carbon tetrahalide or triphenylphosphin together with molecular halogen or carbonyl dihalides or sulfinyl dihalides or sulfonyl dihalides or para-toluenesulfonyl chloride. In the last reaction step compounds XVII are reacted via animation to obtain compounds II, wherein R^{a2} is X, which is chloro or bromo. This reaction is preferably effected either in presence of potassium phtalimide followed by liberating the amine with hydrazine or ethanol amine or in presence of sodium diformyl amide followed by presence of HCl.

A further method to build up compounds II by nitrosylation is shown below, wherein X′ is alkyl, preferably butyl:

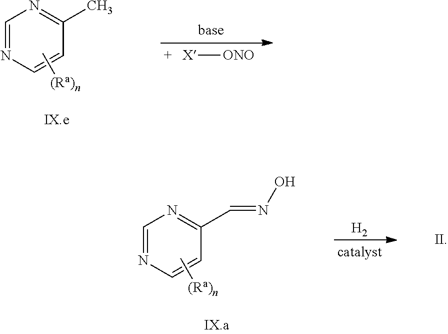

Methyl compounds IX.e can be reacted with alkyl nitrites in the presence of an organic base such as potassium methanolate to obtain oxime compounds IX.a. Compounds IX.a can be reacted with moelcular hydrogen preferably in presence of a catalyst to obtain corresponding amine compounds II.

Sulfonic acid compounds III are known from prior art or can be obtained according to procedures known in the art.

A suitable method to build up compounds III, wherein Het, A and Y are as defined above and L is chlorine is shown below:

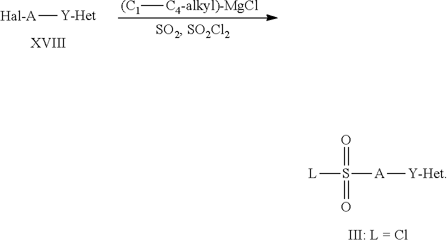

A further suitable method to build up compounds III, wherein A is as described herein and preferable A is 1,4-phenylene, is shown below:

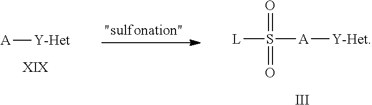

Sulfonation of compound XIX with pyridine-SO₃ or dioxane-SO₃ complex affords compound III, wherein L is OH (for sulfonation procedure cf. Mizuno, A. et. al., Tetrahedron Lett. 41, 6605, 2000). Sulfonation of compound XIX with oleum under heating affords compound III, wherein L is OH, as well (cf. U.S. Pat. No. 4,874,894). Sulfonation of compound XXI with chlorosulfonic acid affords compound III, wherein L is CI (cf. WO 03/055857, WO 03/016313 or WO 02/64593).

Compounds XIX are known from prior art or can be obtained according to procedures known in the art.

A suitable method to build up compounds XIX, wherein Y is O, is shown below:

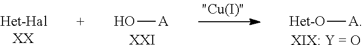

Reaction of a halogen substituted heterocyclic compound XX with a cyclic alcohol XXI in the presence of a Cu(I) salt and optionally in presence of a basic substance affords heteroaryl cyclyl ethers XXI, wherein Y is —O—. This reaction in presence of Cu(I) catalysts is known from prior art.

A further method to build up compounds III via sulfohalogenation is shown below, wherein L is a leaving around as defined above:

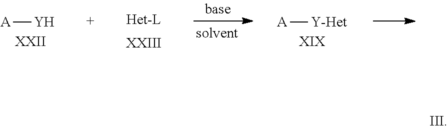

Compounds XXII can be reacted with heteroaryl compounds XXIII advantageously in presence of a base and a solvent to obtain compounds XIX, which can be converted to compounds III via sulfohalogenation in the presence of sulfonic acid derivatives such as CISO₃H, SO₂Cl₂, H₂SO₄ and advantageously in the presence of phosphous trichloride or phosphorous pentachloride. The sulfohalogenation reaction step may also performed in two consecutive steps, wherein the sulfonation is performed first with sulfonic acid and yields a compound III, wherein L is hydroxy, followed by the halogenation in presence of customary halogenation agents such as POCl₃, SO₂Cl₂, SOC₂ and COCl₂. The sulfonation reaction can be performed for example in analogy to methods described in Zhongnan Minzu Daxue Xuebao, Ziran Kexueban 25(4), 28-30, 2006; J. Med. Chem. 44(21), 3488-3503, 2001; or J. Med. Chem. 44(21), 3488-3503, 2001. The halogenation reaction can be performed for example in analogy to methods described in WO 07/149730; Eur. J. Org. Chem. (22), 3669-3675, 2007; Eur. J. Org. Chem. (22), 3669-3675, 2007; Huaxue Shijie 45(1), 29-31, 25, 2004.

A further method to build up compounds III via a Sandmeyer reaction is shown below, wherein L is a leaving group as defined above:

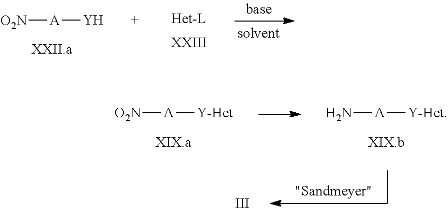

Nitro derivatives of compounds XXII (herein referred to as XXII.a) can be reacted preferably in presence of a base and a solvent with compounds XXIII via nucleophilic aromatic substitution to yield nitro derivatives of compounds XIX (herein referred to as XIX.a). The nitro compounds XIX.a can reduced with customary reducing agents to obtain the amine derivatives XIX.b, advantageously in the presence of a catalyst (Ni, Pd, Pt). These reactions are known from prior art. The amine derivatives XIX.b can reacted via a Sandmeyer reaction in presence of a mineral acid and a metal nitrite, preferably an alkali metal nitrite, followed by the presence of copper halide and stoichiometric amounts of sulfur dioxide to obtain compounds III. The Sandmeyer reaction can be performed for example in analogy to methods described in Chem. Commun. 44, 4620-4622, 2006; WO 06/44732; J. Med. Chem. 48(23), 7363-7373, 2005; or WO 05/118529.

A further method to build up compounds III via oxidation of sulfur is shown below, wherein L is a leaving group as defined above and Z is hydrogen or C₁-C₄-alkyl:

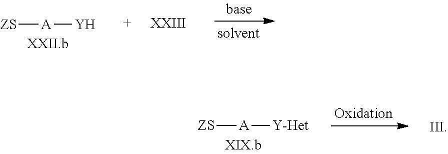

Thiol or thioether derivatives of compounds XXII (herein referred to as XXII.b) can be reacted preferably in presence of a base and a solvent with compounds XXIII to yield thiol or thioether derivatives of compounds XIX (herein referred to as XIX.b). The sulfide derivatives XIX.b can be oxidized in the presence of suitable oxidizing agents such as NaOCl, oxygen or chlorine to obtain compounds III. This reaction is usually carried out in a solvent. Suitable solvents are halogenated hydrocarbons, such as DCM, chloroform, and chlorobenzene, nitriles, such as acetonitrile and proprionitrile, water and acetic acid. Preference is given to acetic acid, water, DCM, chlorobenzene or acetonitrile and mixtures thereof.

Alternatively, compounds III can also be obtained via oxidation of sulfur as shown below, wherein Z is hydrogen or C₁-C₄-alkyl and L is a leaving group as defined above and p is 1 or 2:

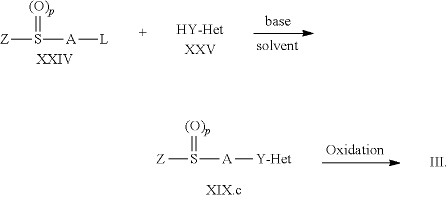

Compounds XXIV can be reacted preferably in presence of a base and a solvent with heteroaryl compounds XXV to yield sulfone or sulfoxide derivatives of compounds XIX (herein referred to as XIX.c). The compounds XIX.c can be oxidized to obtain compounds III using the conditions for the oxidation of compounds XIX.b as described above.

A further method to build up compounds III via oxidation of sulfur is shown below, wherein Het, A, Y, L and Z are as defined above:

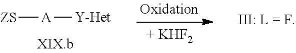

The thiol or thioether derivatives XIX.b can be oxidized in the presence of suitable oxidizing agents agents such as chlorine in the presence of potassium bifluoride to obtain sulfofluoride compounds III, wherein L is fluoro. This reaction can be performed for example in analogy to methods described in J. Org. Chem. 72(15), 5847-5850, 2007; U.S. Pat. No. 4,454,135; Arch. Pharm. 323(2), 83-7, 1990; Synth. Commun. 25(18), 2813-17, 1995; J. Am. Chem. Soc. 78, 5008-11, 1956; U.S. Pat. No. 4,521,241; J. Org. Chem. 61(26), 9289-9292, 1996; J. Med. Chem. 46(12), 2376-2396, 2003; J. Org. Chem. 71(3), 1080-1084, 2006; or J. Med. Chem. 48(20), 6326-6339, 2005.

Alternatively, sulfofluoride compounds III, wherein L is fluoro, can also be obtained via fluorination of sulfochloride compounds III, wherein, L is chloro, in the presence of fluorides Mt-Fₚ, wherein p is 1 or 2 and Mt is a metal cation, preferably K, Na or Ca, as shown below, wherein Het, Y and A are as defined above:

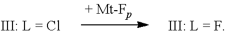

This reaction can performed for example in analogy to methods described in WO 07/142266; Bioorg. Med. Chem. Lett. 17(13), 3760-3764, 2007; J. Fluorine Chem. 31(3), 319-32, 1986; J. Chem. Soc., Chem. Commun. (10), 793-4, 1986; or J. Am. Chem. Soc. 76, 3230-2, 1954.

A method to activate compounds III, wherein L is fluoro or chloro, is shown below, wherein Ar is a heteroaryl or phenyl radical, preferably pentafluorphenyl or hydroxybenzotriazolyl:

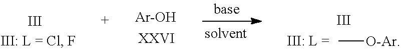

To obtain activated sulfonic acid phenyl ester derivatives of sulfohalide compounds III, compounds III can be reacted with compounds XXVI, wherein Ar is a heteroaryl or phenyl radical, preferably pentafluorophenyl or hydroxybenzotriazolyl, advantageously in presence of a solvent and a basic substance in analogy to methods described in J. Biol. Chem. 217, 107-10, 1955; Zhurnal Obshchei Khimii 30, 479-83, 1960; or J. Org. Chem. 42(20), 3265-70. 1977.

Alternatively, compounds III, wherein L is hydroxy, can be reacted with compounds XXVI, wherein and Ar is as defined above, to obtain activated sulfonic acid phenyl ester derivatives of compounds III, as shown below:

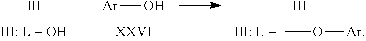

The reaction can be carried out advantageously in presence of triphenylphosphine oxide and/or triflic anhydride in analogy to methods described in J. Am. Chem. Soc. 126(4), 1024-1025, 2004.

A further method to activate compounds III is shown below:

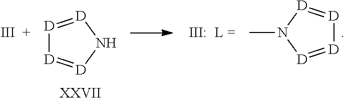

To obtain activated heteroaryl derivatives of compounds III, compounds III can be reacted with heteroaryl compounds XXVII, wherein D is N, CH or CZ, wherein Z is C₁-C₄-alkyl and wherein two adjacent CZ groups may form a fused phenyl ring. The reaction can be carried out advantageously in presence of a solvent in analogy to methods described in Z. Naturforsch., B: Chem. Sci. 56(12), 1360-1368, 2001; or Arch. Pharm. 328(3), 223-9, 1995.

Compounds I and intermediates, wherein R is hydrogen, can be converted by conventional processes such as alkylation. Examples of suitable alkylating agents include alkyl halides, such as alkyl chloride, alkyl bromide or alkyl iodide, examples being methyl chloride, methyl bromide or methyl iodide, or dialkyl sulfates such as dimethyl sulfate or diethyl sulfate. The reaction with the alkylating agent is carried out advantageously in the presence of a solvent. Solvents used for these reactions are—depending on temperature range—aliphatic, cycloaliphatic or aromatic hydrocarbons such as hexane, cyclohexane, toluene, xylene, chlorinated aliphatic and aromatic hydrocarbons such as DCM, chlorobenzene, open-chain dialkyl ethers such as diethyl ether, di-n-propyl ether, MTBE, cyclic ethers such as tetrahydrofuran, 1,4-dioxane, glycol ethers such as dimethyl glycol ether, and also DMSO, DMF, dimethyl acetamide, NMP, NEP and acetic acid ethyl ester, preferably DMF, DMSO, NMP or NEP, or mixtures of these solvents.

The N-oxides may be prepared from the compounds I according to conventional oxidation methods, for example by treating compounds I with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocycl. Chem. 18(7), 1305-8, 1981) or oxone (cf. J. Am. Chem. Soc. 123(25), 5962-5973, 2001). The oxidation may lead to pure mono-N-oxides or to a mixture of different N-oxides, which can be separated by conventional methods such as chromatography.

If individual compounds I cannot be obtained by the routes described above, they can be prepared by derivatization of other compounds I.

If the synthesis yields mixtures of isomers, a separation is generally not necessarily required since in some cases the individual isomers can be interconverted during workup for use or during application (for example under the action of light, acids or bases). Such conversions may also take place after use, for example in the treatment of plants in the treated plant, or in the harmful fungus to be controlled.

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or goose-berries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grape-fruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; natural rubber plants or ornamental and forestry plants, such as flowers, shrubs, broad-leaved trees or evergreens, e. g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as hydroxyphenylpyruvate dioxygenase (HPPD) inhibitors; acetolactate synthase (ALS) inhibitors, such as sulfonyl ureas (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e. g. U.S. Pat. No. 6,222,100, WO 01/82685, WO 00/026390, WO 97/41218, WO 98/002526, WO 98/02527, WO 04/106529, WO 05/20673, WO 03/014357, WO 03/13225, WO 03/14356, WO 04/16073); enolpyruvylshikimate-3-phosphate synthase (EPSPS) inhibitors, such as glyphosate (see e. g. WO 92/00377); glutamine synthetase (GS) inhibitors, such as glufosinate (see e.g. EP-A 242 236, EP-A 242 246) or oxynil herbicides (see e. g. U.S. Pat. No. 5,559,024) as a result of conventional methods of breeding or genetic engineering. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.) and LibertyLink® (glufosinatetolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The compounds I and compositions thereof, respectively, are particularly suitable for controlling the following plant diseases:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternate), tomatoes (e. g. A. solani or A. alternate) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e.g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e.g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani on soybeans and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grain-staining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, and asparagus (e. g. P. asparagi); Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

The compounds I and compositions thereof, resepctively, are also suitable for controlling harmful fungi in the protection of materials (e. g. wood, paper, paint dispersions, fiber or fabrics) and in the protection of stored products. As to the protection of wood and construction materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichorma spp., Alternaria spp., Paecilomyces spp. and Zygomycetes such as Mucor spp., and in addition in the protection of stored products the following yeast fungi are worthy of note: Candida spp. and Saccharomyces cerevisae. The compounds I and compositions thereof, resepectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress.The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising a solvent or solid carrier and at least one compound I and to the use for controlling harmful fungi.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes and granules. The composition type depends on the particular intended purpose; in each case, it should ensure a fine and uniform distribution of the compound according to the invention.

Examples for composition types are suspensions (SC, OD, FS), pastes, pastilles, wettable powders or dusts (WP, SP, SS, WS, DP, DS) or granules (GR, FG, GG, MG), which can be water-soluble or wettable, as well as gel formulations for the treatment of plant propagation materials such as seeds (GF).

Usually the composition types (e. g. SC, OD, FS, WG, SG, WP, SP, SS, WS, GF) are employed diluted. Composition types such as DP, DS, GR, FG, GG and MG are usually used undiluted.

The compositions are prepared in a known manner (cf. U.S. Pat. No. 3,060,084, EP-A 707 445 (for liquid concentrates), Browning: “Agglomeration”, Chemical Engineering, Dec. 4, 1967, 147-48, Perry's Chemical Engineer's Handbook, 4th Ed., McGraw-Hill, New York, 1963, S. 8-57 and ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8th Ed., Blackwell Scientific, Oxford, 1989) and Mollet, H. and Grubemann, A.: Formulation technology (Wiley VCH Verlag, Weinheim, 2001).

The agrochemical compositions may also comprise auxiliaries which are customary in agrochemical compositions. The auxiliaries used depend on the particular application form and active substance, respectively.

Examples for suitable auxiliaries are solvents, solid carriers, dispersants or emulsifiers (such as further solubilizers, protective colloids, surfactants and adhesion agents), organic and anorganic thickeners, bactericides, anti-freezing agents, anti-foaming agents, if appropriate colorants and tackifiers or binders (e. g. for seed treatment formulations).

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e. g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e. g. amines such as N-methylpyrrolidone.

Solid carriers are mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e. g., ammonium sulfate, ammonium phosphate, ammonium nitrate, ureas, and products of vegetable origin, such as cereal meal, tree bark meal, wood meal and nutshell meal, cellulose powders and other solid carriers.

Suitable surfactants (adjuvants, wtters, tackifiers, dispersants or emulsifiers) are alkali metal, alkaline earth metal and ammonium salts of aromatic sulfonic acids, such as ligninsoulfonic acid (Borresperse® types, Borregard, Norway) phenolsulfonic acid, naphthalenesulfonic acid (Morwet® types, Akzo Nobel, U.S.A.), dibutylnaphthalenesulfonic acid (Nekal® types, BASF, Germany),and fatty acids, alkylsulfonates, alkylarylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and proteins, denatured proteins, polysaccharides (e. g. methylcellulose), hydrophobically modified starches, polyvinyl alcohols (Mowiol® types, Clariant, Switzerland), polycarboxylates (Sokolan® types, BASF, Germany), polyalkoxylates, polyvinylamines (Lupasol® types, BASF, Germany), polyvinylpyrrolidone and the copolymers therof.

Examples for thickeners (i. e. compounds that impart a modified flowability to compositions, i. e. high viscosity under static conditions and low viscosity during agitation) are polysaccharides and organic and anorganic clays such as Xanthan gum (Kelzan®, CP Kelco, U.S.A.), Rhodopol® 23 (Rhodia, France), Veegum® (R.T. Vanderbilt, U.S.A.) or Attaclay® (Engelhard Corp., NJ, USA).

Bactericides may be added for preservation and stabilization of the composition. Examples for suitable bactericides are those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie).

Examples for suitable anti-freezing agents are ethylene glycol, propylene glycol, urea and glycerin.

Examples for anti-foaming agents are silicone emulsions (such as e. g. Silikon® SRE, Wacker, Germany or Rhodorsil®, Rhodia, France), long chain alcohols, fatty acids, salts of fatty acids, fluoroorganic compounds and mixtures thereof.

Suitable colorants are pigments of low water solubility and water-soluble dyes. Examples to be mentioned and the designations rhodamin B, C. I. pigment red 112, C. I. solvent red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples for tackifiers or binders are polyvinylpyrrolidons, polyvinylacetates, polyvinyl alcohols and cellulose ethers (Tylose®, Shin-Etsu, Japan).

Powders, materials for spreading and dusts can be prepared by mixing or concomitantly grinding the compounds I and, if appropriate, further active substances, with at least one solid carrier.

Granules, e. g. coated granules, impregnated granules and homogeneous granules, can be prepared by binding the active substances to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e. g., ammonium sulfate, ammonium phosphate, ammonium nitrate, ureas, and products of vegetable origin, such as cereal meal, tree bark meal, wood meal and nutshell meal, cellulose powders and other solid carriers.

### Examples for Composition Types Are:

1. Composition Types for Dilution with Water

i) Water-Soluble Concentrates (SL, LS)

10 parts by weight of a compound I according to the invention are dissolved in 90 parts by weight of water or in a water-soluble solvent. As an alternative, wetting agents or other auxiliaries are added. The active substance dissolves upon dilution with water. In this way, a composition having a content of 10% by weight of active substance is obtained.

ii) Dispersible Concentrates (DC)

20 parts by weight of a compound I according to the invention are dissolved in 70 parts by weight of cyclohexanone with addition of 10 parts by weight of a dispersant, e. g. polyvinylpyrrolidone. Dilution with water gives a dispersion. The active substance content is 20% by weight.

iii) Emulsifiable Concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The composition has an active substance content of 15% by weight.

iv) Emulsions (EW, EO, ES)

25 parts by weight of a compound I according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The composition has an active substance content of 25% by weight.

v) Suspensions (SC, OD, FS)

In an agitated ball mill, 20 parts by weight of a compound I according to the invention are comminuted with addition of 10 parts by weight of dispersants and wetting agents and 70 parts by weight of water or an organic solvent to give a fine active substance suspension. Dilution with water gives a stable suspension of the active substance. The active substance content in the composition is 20% by weight.

vi) Water-Dispersible Granules and Water-Soluble Granules (WG, SG)

50 parts by weight of a compound I according to the invention are ground finely with addition of 50 parts by weight of dispersants and wetting agents and prepared as water-dispersible or water-soluble granules by means of technical appliances (e. g. extrusion, spray tower, fluidized bed). Dilution with water gives a stable dispersion or solution of the active substance. The composition has an active substance content of 50% by weight.

vii) Water-Dispersible Powders and Water-Soluble Powders (WP, SP, SS, WS)

75 parts by weight of a compound I according to the invention are ground in a rotor-stator mill with addition of 25 parts by weight of dispersants, wetting agents and silica gel. Dilution with water gives a stable dispersion or solution of the active substance. The active substance content of the composition is 75% by weight.

viii) Gel (GF)

In an agitated ball mill, 20 parts by weight of a compound I according to the invention are comminuted with addition of 10 parts by weight of dispersants, 1 part by weight of a gelling agent wetters and 70 parts by weight of water or of an organic solvent to give a fine suspension of the active substance. Dilution with water gives a stable suspension of the active substance, whereby a composition with 20% (w/w) of active substance is obtained.

### 2. Composition Types to be Applied Undiluted

ix) Dustable Powders (DP, DS)

5 parts by weight of a compound I according to the invention are ground finely and mixed intimately with 95 parts by weight of finely divided kaolin. This gives a dustable composition having an active substance content of 5% by weight.

x) Granules (GR, FG, GG, MG)

0.5 parts by weight of a compound I according to the invention is ground finely and associated with 99.5 parts by weight of carriers. Current methods are extrusion, spray-drying or the fluidized bed. This gives granules to be applied undiluted having an active substance content of 0.5% by weight.

xi) ULV Solutions (UL)

10 parts by weight of a compound I according to the invention are dissolved in 90 parts by weight of an organic solvent, e. g. xylene. This gives a composition to be applied undiluted having an active substance content of 10% by weight.

The agrochemical compositions generally comprise between 0.01 and 95%, preferably between 0.1 and 90%, most preferably between 0.5 and 90%, by weight of active substance. The active substances are employed in a purity of from 90% to 100%, preferably from 95% to 100% (according to NMR spectrum).

Water-soluble concentrates (LS), flowable concentrates (FS), powders for dry treatment (DS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds. These compositions can be applied to plant propagation materials, particularly seeds, diluted or undiluted. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying or treating agrochemical compounds and compositions thereof, respectively, on to plant propagation material, especially seeds, are known in the art, and include dressing, coating, pelleting, dusting, soaking and in-furrow application methods of the propagation material. In a preferred embodiment, the compounds or the compositions thereof, respectively, are applied on to the plant propagation material by a method such that germination is not induced, e. g. by seed dressing, pelleting, coating and dusting.

In a preferred embodiment, a suspension-type (FS) composition is used for seed treatment. Typcially, a FS composition may comprise 1-800 g/l of active substance, 1-200 g/l Surfactant, 0 to 200 g/l antifreezing agent, 0 to 400 g/l of binder, 0 to 200 g/l of a pigment and up to 1 liter of a solvent, preferably water.

The active substances can be used as such or in the form of their compositions, e. g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring. The application forms depend entirely on the intended purposes; it is intended to ensure in each case the finest possible distribution of the active substances according to the invention.

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1% by weight of active substance.

The active substances may also be used successfully in the ultra-low-volume process (ULV), it being possible to apply compositions comprising over 95% by weight of active substance, or even to apply the active substance without additives.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required.

When used in the protection of materials or stored products, the amount of active substance applied depends on the kind of application area and on the desired effect. Amounts customarily applied in the protection of materials are, e. g., 0.001 g to 2 kg, preferably 0.005 g to 1 kg, of active substance per cubic meter of treated material.

Various types of oils, wetters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, e. g. Pluronic RPE 2035® and Genapol B®; alcohol ethoxylates such as Lutensol XP 80®; and dioctyl sulfosuccinate sodium such as Leophen RA®.

The compositions according to the invention can, in the use form as fungicides, also be present together with other active substances, e. g. with herbicides, insecticides, growth regulators, fungicides or else with fertilizers, as pre-mix or, if appropriate, not until immeadiately prior to use (tank mix).

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of active substances, in conjunction with which the compounds according to the invention can be used, is intended to illustrate the possible combinations but does not limit them:
A) strobilurins
   azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methyl-phenoxy)-5-fluoro-pyrimidin-4-yloxy)-phenyl)-2-methoxyimino-N-methyl-acetamide, 3-methoxy-2-(2-(N-(4-methoxy-phenyl)-cyclopropane-carboximidoylsulfanylmethyl)-phenyl)-acrylic acid methyl ester, methyl (2-chloro-5-[1-(3-methylbenzyloxyimino)ethyl]benzyl)carbamate and 2-(2-(3-(2,6-dichlorophenyl)-1-methyl-allylideneaminooxymethyl)-phenyl)-2-methoxyimino-N-methyl-acetamide;
B) carboxamides
   carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methyl-thiazole-5-carboxanilide, 2-chloro-N-(1,1,3-trimethyl-indan-4-yl)-nicotinamide, N-(3′,4′,5′-trifluorobiphenyl-2-yl)-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3-dimethyl-butyl)-phenyl)-1,3-dimethyl-5-fluoro-1H-pyrazole-4-carboxamide and N-(2-(1,3,3-trimethyl-butyl)-phenyl)-1,3-dimethyl-5-fluoro-1H-pyrazole-4-carboxamide;
   carboxylic morpholides: dimethomorph, flumorph, pyrimorph;
   benzoic acid amides: flumetover, fluopicolide, fluopyram, zoxamide, N-(3-Ethyl-3,5,5-trimethyl-cyclohexyl)-3-formylamino-2-hydroxy-benzamide;
   other carboxamides: carpropamid, dicyclomet, mandiproamid, oxytetracyclin, silthiofarm and N-(6-methoxy-pyridin-3-yl) cyclopropanecarboxylic acid amide;
C) azoles
   triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chloro-phenyl)-2-([1,2,4]triazol-1-yl)-cycloheptanol;
   imidazoles: cyazofamid, imazalil, pefurazoate, prochloraz, triflumizol;
   benzimidazoles: benomyl, carbendazim, fuberidazole, thiabendazole;
   others: ethaboxam, etridiazole, hymexazole and 2-(4-chloro-phenyl)-N-[4-(3,4-dimethoxy-phenyl)-isoxazol-5-yl]-2-prop-2-ynyloxy-acetamide;
D) heterocyclic compounds
   pyridines: fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine, 2,3,5,6-tetra-chloro-4-methanesulfonyl-pyridine, 3,4,5-trichloropyridine-2,6-di-carbonitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide;
   pyrimidines: bupirimate, cyprodinil, diflumetorim, fenarimol, ferimzone, mepanipyrim, nitrapyrin, nuarimol, pyrimethanil;
   piperazines: triforine;
   pyrroles: fenpiclonil, fludioxonil;
   morpholines: aldimorph, dodemorph, dodemorph-acetate, fenpropimorph, tridemorph;
   piperidines: fenpropidin;
   dicarboximides: fluoroimid, iprodione, procymidone, vinclozolin;
   non-aromatic 5-membered heterocycles: famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester;
   others: acibenzolar-S-methyl, amisulbrom, anilazin, blasticidin-S, captafol, captan, chinomethionat, dazomet, debacarb, diclomezine, difenzoquat, difenzoquat-methylsulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole, 5-chloro-7-(4-methylpiperidin-1-yl)-6-(2,4,6-trifluorophenyl)-[1,2,4]triazolo[1,5-a]pyrimidine and 5-ethyl-6-octyl-[1,2,4]triazolo[1,5-a]pyrimidine-7-ylamine;
E) carbamates
   thio- and dithiocarbamates: ferbam, mancozeb, maneb, metam, methasulphocarb, metiram, propineb, thiram, zineb, ziram;
   carbamates: benthiavalicarb, diethofencarb, iprovalicarb, propamocarb, propamocarb hydrochlorid, valiphenal and N-(1-(1-(4-cyano-phenyl)ethanesulfonyl)-but-2-yl) carbamic acid-(4-fluorophenyl)ester;
F) other active substances
   guanidines: guanidine, dodine, dodine free base, guazatine, guazatine-acetate, iminoctadine, iminoctadine-triacetate, iminoctadine-tris(albesilate);
   antibiotics: kasugamycin, kasugamycin hydrochloride-hydrate, streptomycin, polyoxine, validamycin A;
   nitrophenyl derivates: binapacryl, dinobuton, dinocap, nitrthal-isopropyl, tecnazen, organometal compounds: fentin salts, such as fentin-acetate, fentin chloride or fentin hydroxide;
   sulfur-containing heterocyclyl compounds: dithianon, isoprothiolane;
   organophosphorus compounds: edifenphos, fosetyl, fosetyl-aluminum, iprobenfos, phosphorous acid and its salts, pyrazophos, tolclofos-methyl;
   organochlorine compounds: chlorothalonil, dichlofluanid, dichlorophen, flusulfamide, hexachlorobenzene, pencycuron, pentachlorphenole and its salts, phthalide, quintozene, thiophanate-methyl, tolylfluanid, N-(4-chloro-2-nitro-phenyl)-N-ethyl-4-methylbenzenesulfonamide;
   inorganic active substances: Bordeaux mixture, copper acetate, copper hydroxide, copper oxychloride, basic copper sulfate, sulfur;
   others: biphenyl, bronopol, cyflufenamid, cymoxanil, diphenylamin, metrafenone, mildiomycin, oxin-copper, prohexadione-calcium, spiroxamine, tolylfluanid, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethyl-silanyl-propoxy)-phenyl)-N-ethyl-N-methyl formamidine, N′-(5-difluoromethyl-2-methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-ylythiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-ylythiazole-4-carboxylic acid methyl-(R)-1,2,3,4-tetrahydro-naphthalen-1-yl-amide, acetic acid 6-tert.-butyl-8-fluoro-2,3-dimethyl-quinolin-4-yl ester and methoxy-acetic acid 6-tert-butyl-8-fluoro-2,3-dimethyl-quinolin-4-yl ester.
G) growth regulators
   abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid , trinexapac-ethyl and uniconazole;
H) herbicides
   acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
   amino acid derivatives: bilanafos, glyphosate, glufosinate, sulfosate;
   aryloxyphenoxypropionates: clodinafop, cyhalofop-butyl, fenoxaprop, fluazifop, haloxyfop, metamifop, propaquizafop, quizalofop, quizalofop-P-tefuryl;
   Bipyridyls: diquat, paraquat;
   (thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
   cyclohexanediones: butroxydim, clethodim, cycloxydim, profoxydim, sethoxydim, tepraloxydim, tralkoxydim;
   dinitroanilines: benfluralin, ethalfluralin, oryzalin, pendimethalin, prodiamine, trifluralin;
   diphenyl ethers: acifluorfen, aclonifen, bifenox, diclofop, ethoxyfen, fomesafen, lactofen, oxyfluorfen;
   hydroxybenzonitriles: bomoxynil, dichlobenil, ioxynil;
   imidazolinones: imazamethabenz, imazamox, imazapic, imazapyr, imazaquin, imazethapyr;
   phenoxy acetic acids: clomeprop, 2,4-dichlorophenoxyacetic acid (2,4-D), 2,4-DB, dichlorprop, MCPA, MCPA-thioethyl, MCPB, Mecoprop;
   pyrazines: chloridazon, flufenpyr-ethyl, fluthiacet, norflurazon, pyridate;
   pyridines: aminopyralid, clopyralid, diflufenican, dithiopyr, fluridone, fluroxypyr, picloram, picolinafen, thiazopyr;
   sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-Asulfonyl)-3-(4,6-dimethoxy-pyrimidin-2-yl)urea;
   triazines: ametryn, atrazine, cyanazine, dimethametryn, ethiozin, hexazinone, metamitron, metribuzin, prometryn, simazine, terbuthylazine, terbutryn, triaziflam;
   ureas: chlorotoluron, daimuron, diuron, fluometuron, isoproturon, linuron, methabenzthiazuron, tebuthiuron;
   other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
   others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclo[3.2.1]oct-3-en-2-one, (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methylphenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.
I) insecticides
   organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
   carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
   pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
   insect growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
   nicotinic receptor agonists/antagonists compounds: clothianidin, dinotefuran, imidacloprid, thiamethoxam, nitenpyram, acetamiprid, thiacloprid, 1-(2-chloro-thiazol-5-ylmethyl)-2-nitrimino-3,5-dimethyl-[1,3,5]triazinane;
   GABA antagonist compounds: endosulfan, ethiprole, fipronil, vaniliprole, pyrafluprole, pyriprole, 5-amino-1-(2,6-dichloro-4-methyl-phenyl)-4-sulfinamoyl-1H-pyrazole-3-carbothioic acid amide;
   macrocyclic lactone insecticides: abamectin, emamectin, milbemectin, lepimectin, spinosad, spinetoram;
   mitochondrial electron transport inhibitor (METI) I acaricides: fenazaquin, pyridaben, tebufenpyrad, tolfenpyrad, flufenerim;
   METI II and III compounds: acequinocyl, fluacyprim, hydramethylnon;
   Uncouplers: chlorfenapyr;
   oxidative phosphorylation inhibitors: cyhexatin, diafenthiuron, fenbutatin oxide, propargite;
   moulting disruptor compounds: cryomazine;
   mixed function oxidase inhibitors: piperonyl butoxide;
   sodium channel blockers: indoxacarb, metaflumizone;
   others: benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluron, and pyrifluquinazon.

The present invention furthermore relates to agrochemical compositions comprising a mixture of at least one compound I (component 1) and at least one further active substance useful for plant protection, e. g. selected from the groups A) to I) (component 2), in particular one further fungicide, e. g. one or more fungicide from the groups A) to F), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to F), as described above, is more efficient than combating those fungi with individual compounds I or individual fungicides from groups A) to F). By applying compounds I together with at least one active substance from groups A) to I) a synergistic effect can be obtained, i.e. more then simple addition of the individual effects is obtained (synergistic mixtures).

According to this invention, applying the compounds I together with at least one further active substance is to be understood to denote, that at least one compound of formula I and at least one further active substance occur simultaneously at the site of action (i.e. the harmful fungi to be controlled or their habitats such as infected plants, plant propagation materials, particularly seeds, surfaces, materials or the soil as well as plants, plant propagation materials, particularly seeds, soil, surfaces, materials or rooms to be protected from fungal attack) in a fungicidally effective amount. This can be obtained by applying the compounds I and at least one further active substance simultaneously, either jointly (e. g. as tank-mix) or sperately, or in succession, wherein the time interval between the individual applications is selected to ensure that the active substance applied first still occurs at the site of action in a sufficient amount at the time of application of the further active substance(s). The order of application is not essential for working of the present invention.

In binary mixtures, i.e. compositions according to the invention comprising one compound I (component 1) and one further active substance (component 2), e. g. one active substance from groups A) to I), the weight ratio of component 1 and component 2 generally depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:3 to 3:1.

In ternary mixtures, i.e. compositions according to the invention comprising one compound I (component 1) and a first further active substance (component 2) and a second further active substance (component 3), e. g. two active substances from groups A) to I), the weight ratio of component 1 and component 2 depends from the properties of the active substances used, preferably it is in the range of from 1:50 to 50:1 and particularly in the range of from 1:10 to 10:1, and the weight ratio of component 1 and component 3 preferably is in the range of from 1:50 to 50:1 and particularly in the range of from 1:10 to 10:1.

The components can be used individually or already partially or completely mixed with one another to prepare the composition according to the invention. It is also possible for them to be packaged and used further as combination composition such as a kit of parts.

In one embodiment of the invention, the kits may include one or more, including all, components that may be used to prepare a subject agrochemical composition. E. g., kits may include one or more fungicide component(s) and/or an adjuvant component and/or a insecticide component and/or a growth regulator component and/or a herbicde. One or more of the components may already be combined together or preformulated. In those embodiments where more than two components are provided in a kit, the components may already be combined together and as such are packaged in a single container such as a vial, bottle, can, pouch, bag or canister. In other embodiments, two or more components of a kit may be packaged separately, i. e., not preformulated. As such, kits may include one or more separate containers such as vials, cans, bottles, pouches, bags or canisters, each container containing a separate component for an agrochemical composition. In both forms, a component of the kit may be applied separately from or together with the further components or as a component of a combination composition according to the invention for preparing the composition according to the invention.

The user applies the composition according to the invention usually from a predosage device, a knapsack sprayer, a spray tank or a spray plane. Here, the agrochemical composition is made up with water and/or buffer to the desired application concentration, it being possible, if appropriate, to add further auxiliaries, and the ready-to-use spray liquor or the agrochemical composition according to the invention is thus obtained. Usually, 50 to 500 liters of the ready-to-use spray liquor are applied per hectare of agricultural useful area, preferably 100 to 400 liters.

According to one embodiment, individual components of the composition according to the invention such as parts of a kit or parts of a binary or ternary mixture may be mixed by the user himself in a spray tank and further auxiliaries may be added, if appropriate (tank mix).

In a further embodiment, either individual components of the composition according to the invention or partially premixed components, e. g. components comprising compounds I and/or active substances from the groups A) to I), may be mixed by the user in a spray tank and further auxiliaries and additives may be added, if appropriate (tank mix)

In a further embodiment, either individual components of the composition according to the invention or partially premixed components, e. g. components comprising compounds I and/or active substances from the groups A) to I), can be applied jointly (e. .g. after tankmix) or consecutively.

Preference is also given to mixtures comprising a compound I (component 1) and at least one active substance selected from the strobilurines of group A) (component 2) and particularly selected from azoxystrobin, dimoxystrobin, fluoxastrobin, kresoximmethyl, orysastrobin, picoxystrobin, pyraclostrobin and trifloxystrobin.

Preference is also given to mixtures comprising a compound I (component 1) and at least one active substance selected from the carboxamides of group B) (component 2) and particularly selected from bixafen, boscalid, sedaxane, fenhexamid, metalaxyl, isopyrazam, mefenoxam, ofurace, dimethomorph, flumorph, fluopicolid (picobenzamid), zoxamide, carpropamid, mandipropamid and N-(3′,4′,5′-trifluorobiphenyl-2-yl)-3-difluoromethyl-1-methyl-1H-pyrazole-4-carboxamide.

Preference is given to mixtures comprising a compound of formula I (component 1) and at least one active substance selected from the azoles of group C) (component 2) and particularly selected from cyproconazole, difenoconazole, epoxiconazole, fluquinconazole, flusilazole, flutriafol, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to mixtures comprising a compound I (component 1) and at least one active substance selected from the heterocyclic compounds of group D) (component 2) and particularly selected from fluazinam, cyprodinil, fenarimol, mepanipyrim, pyrimethanil, triforine, fludioxonil, dodemorph, fenpropimorph, tridemorph, fenpropidin, iprodione, vinclozolin, famoxadone, fenamidone, probenazole, proquinazid, acibenzolar-S-methyl, captafol, folpet, fenoxanil, quinoxyfen and 5-ethyl-6-octyl-[1,2,4]triazolo[1,5-a]pyrimidine-7-ylamine.

Preference is also given to mixtures comprising a compound I (component 1) and at least one active substance selected from the carbamates of group E) (component 2) and particularly selected from mancozeb, metiram, propineb, thiram, iprovalicarb, benthiavalicarb and propamocarb.

Preference is also given to mixtures comprising a compound I (component 1) and at least one active substance selected from the fungicides given in group F) (component 2) and particularly selected from dithianon, fentin salts, such as fentin acetate, fosetyl, fosetyl-aluminium, H₃PO₃ and salts thereof, chlorthalonil, dichlofluanid, thiophanatmethyl, copper acetate, copper hydroxide, copper oxychloride, copper sulfate, sulfur, cymoxanil, metrafenone and spiroxamine.

Accordingly, the present invention furthermore relates to compositions comprising one compound I (component 1) and one further active substance (component 2), which further active substance is selected from the column “Component 2” of the lines B-1 to B-346 of Table B.

A further embodiment relates to the compositions B-1 to B-346 listed in Table B, where a row of Table B corresponds in each case to a fungicidal composition comprising one of the in the present specification individualized compounds of formula I (component 1) and the respective further active substance from groups A) to I) (component 2) stated in the row in question. Preferably, the compositions described comprise the active substances in synergistically effective amounts.

TABLE B
Composition comprising one indiviualized compound I
and one further active substance from groups A) to I)

| Mixture | Component 1 | Component 2 |
|---|---|---|
| B-1 | one individualized compound I | Azoxystrobin |
| B-2 | one individualized compound I | Dimoxystrobin |
| B-3 | one individualized compound I | Enestroburin |
| B-4 | one individualized compound I | Fluoxastrobin |
| B-5 | one individualized compound I | Kresoxim-methyl |
| B-6 | one individualized compound I | Metominostrobin |
| B-7 | one individualized compound I | Orysastrobin |
| B-8 | one individualized compound I | Picoxystrobin |
| B-9 | one individualized compound I | Pyraclostrobin |
| B-10 | one individualized compound I | Pyribencarb |
| B-11 | one individualized compound I | Trifloxystrobin |
| B-12 | one individualized compound I | 2-(2-(6-(3-Chloro-2-methyl-phenoxy)- |
| | | 5-fluoro-pyrimidin-4-yloxy)-phenyl)-2- |
| | | methoxyimino-N-methyl-acetamide |
| B-13 | one individualized compound I | 2-(ortho-((2,5-Dimethylphenyl-oxy- |
| | | methylen)phenyl)-3-methoxy- |
| | | acrylsäuremethylester |
| B-14 | one individualized compound I | 3-Methoxy-2-(2-(N-(4-methoxy-phenyl)- |
| | | cyclopropanecarboximidoylsulfanyl- |
| | | methyl)-phenyl)-acrylic acid methyl ester |
| B-15 | one individualized compound I | 2-(2-(3-(2,6-dichlorophenyl)-1-methyl- |
| | | allylideneaminooxymethyl)-phenyl)- |
| | | 2-methoxyimino-N-methyl-acetamide |
| B-16 | one individualized compound I | Benalaxyl |
| B-17 | one individualized compound I | Benalaxyl-M |
| B-18 | one individualized compound I | Benodanil |
| B-19 | one individualized compound I | Bixafen |
| B-20 | one individualized compound I | Boscalid |
| B-21 | one individualized compound I | Carboxin |
| B-22 | one individualized compound I | Fenfuram |
| B-23 | one individualized compound I | Fenhexamid |
| B-24 | one individualized compound I | Flutolanil |
| B-25 | one individualized compound I | Furametpyr |
| B-26 | one individualized compound I | Isopyrazam |
| B-27 | one individualized compound I | Isotianil |
| B-28 | one individualized compound I | Kiralaxyl |
| B-29 | one individualized compound I | Mepronil |
| B-30 | one individualized compound I | Metalaxyl |
| B-31 | one individualized compound I | Metalaxyl-M |
| B-32 | one individualized compound I | Ofurace |
| B-33 | one individualized compound I | Oxadixyl |
| B-34 | one individualized compound I | Oxycarboxin |
| B-35 | one individualized compound I | Penthiopyrad |
| B-36 | one individualized compound I | Sedaxane |
| B-37 | one individualized compound I | Tecloftalam |
| B-38 | one individualized compound I | Thifluzamide |
| B-39 | one individualized compound I | Tiadinil |
| B-40 | one individualized compound I | 2-Amino-4-methyl-thiazole-5-carboxylic |
| | | acid anilide |
| B-41 | one individualized compound I | 2-Chloro-N-(1,1,3-trimethyl-indan-4-yl)- |
| | | nicotinamide |
| B-42 | one individualized compound I | N-(3′,4′,5′-trifluorobiphenyl-2-yl)-3-di- |
| | | fluoromethyl-1-methyl-1H-pyrazole- |
| | | 4-carboxamide |
| B-43 | one individualized compound I | N-(4′-trifluoromethylthiobiphenyl-2-yl)- |
| | | 3-difluoromethyl-1-methyl-1H-pyrazole- |
| | | 4-carboxamide |
| B-44 | one individualized compound I | N-(2-(1,3-dimethyl-butyl)-phenyl)- |
| | | 1,3-dimethyl-5-fluoro-1H-pyrazole- |
| | | 4-carboxamide |
| B-45 | one individualized compound I | N-(2-(1,3,3-trimethyl-butyl)-phenyl)- |
| | | 1,3-dimethyl-5-fluoro-1H-pyrazole- |
| | | 4-carboxamide |
| B-46 | one individualized compound I | Dimethomorph |
| B-47 | one individualized compound I | Flumorph |
| B-48 | one individualized compound I | Pyrimorph |
| B-49 | one individualized compound I | Flumetover |
| B-50 | one individualized compound I | Fluopicolide |
| B-51 | one individualized compound I | Fluopyram |
| B-52 | one individualized compound I | Zoxamide |
| B-53 | one individualized compound I | N-(3-Ethyl-3,5,5-trimethyl-cyclohexyl)- |
| | | 3-formylamino-2-hydroxy-benzamide |
| B-54 | one individualized compound I | Carpropamid |
| B-55 | one individualized compound I | Diclocymet |
| B-56 | one individualized compound I | Mandipropamid |
| B-57 | one individualized compound I | Oxytetracyclin |
| B-58 | one individualized compound I | Silthiofam |
| B-59 | one individualized compound I | N-(6-methoxy-pyridin-3-yl) cyclopro- |
| | | panecarboxylic acid amide |
| B-60 | one individualized compound I | Azaconazole |
| B-61 | one individualized compound I | Bitertanol |
| B-62 | one individualized compound I | Bromuconazole |
| B-63 | one individualized compound I | Cyproconazole |
| B-64 | one individualized compound I | Difenoconazole |
| B-65 | one individualized compound I | Diniconazole |
| B-66 | one individualized compound I | Diniconazole-M |
| B-67 | one individualized compound I | Epoxiconazole |
| B-68 | one individualized compound I | Fenbuconazole |
| B-69 | one individualized compound I | Fluquinconazole |
| B-70 | one individualized compound I | Flusilazole |
| B-71 | one individualized compound I | Flutriafol |
| B-72 | one individualized compound I | Hexaconazol |
| B-73 | one individualized compound I | Imibenconazole |
| B-74 | one individualized compound I | Ipconazole |
| B-75 | one individualized compound I | Metconazole |
| B-76 | one individualized compound I | Myclobutanil |
| B-77 | one individualized compound I | Oxpoconazol |
| B-78 | one individualized compound I | Paclobutrazol |
| B-79 | one individualized compound I | Penconazole |
| B-80 | one individualized compound I | Propiconazole |
| B-81 | one individualized compound I | Prothioconazole |
| B-82 | one individualized compound I | Simeconazole |
| B-83 | one individualized compound I | Tebuconazole |
| B-84 | one individualized compound I | Tetraconazole |
| B-85 | one individualized compound I | Triadimefon |
| B-86 | one individualized compound I | Triadimenol |
| B-87 | one individualized compound I | Triticonazole |
| B-88 | one individualized compound I | Uniconazole |
| B-89 | one individualized compound I | 1-(4-Chloro-phenyl)-2-([1,2,4]triazol-1- |
| | | yl)-cycloheptanol |
| B-90 | one individualized compound I | Cyazofamid |
| B-91 | one individualized compound I | Imazalil |
| B-92 | one individualized compound I | Imazalil-sulfate |
| B-93 | one individualized compound I | Pefurazoate |
| B-94 | one individualized compound I | Prochloraz |
| B-95 | one individualized compound I | Triflumizole |
| B-96 | one individualized compound I | Benomyl |
| B-97 | one individualized compound I | Carbendazim |
| B-98 | one individualized compound I | Fuberidazole |
| B-99 | one individualized compound I | Thiabendazole |
| B-100 | one individualized compound I | Ethaboxam |
| B-101 | one individualized compound I | Etridiazole |
| B-102 | one individualized compound I | Hymexazole |
| B-103 | one individualized compound I | 2-(4-Chloro-phenyl)-N-[4-(3,4-dimeth- |
| | | oxy-phenyl)-isoxazol-5-yl]-2-prop-2-yn- |
| | | yloxy-acetamide |
| B-104 | one individualized compound I | Fluazinam |
| B-105 | one individualized compound I | Pyrifenox |
| B-106 | one individualized compound I | 3-[5-(4-Chloro-phenyl)-2,3-dimethyl- |
| | | isoxazolidin-3-yl]-pyridine |
| B-107 | one individualized compound I | 3-[5-(4-Methyl-phenyl)-2,3-dimethyl- |
| | | isoxazolidin-3-yl]-pyridine |
| B-108 | one individualized compound I | 2,3,5,6-Tetrachloro-4-methanesulfonyl- |
| | | pyridine |
| B-109 | one individualized compound I | 3,4,5-Trichloro-pyridine-2,6- |
| | | dicarbonitrile |
| B-110 | one individualized compound I | N-(1-(5-Bromo-3-chloro-pyridin-2-yl)- |
| | | ethyl)-2,4-dichloro-nicotinamide |
| B-111 | one individualized compound I | N-((5-Bromo-3-chloro-pyridin-2-yl)- |
| | | methyl)-2,4-dichloro-nicotinamide |
| B-112 | one individualized compound I | Bupirimate |
| B-113 | one individualized compound I | Cyprodinil |
| B-114 | one individualized compound I | Diflumetorim |
| B-115 | one individualized compound I | Fenarimol |
| B-116 | one individualized compound I | Ferimzone |
| B-117 | one individualized compound I | Mepanipyrim |
| B-118 | one individualized compound I | Nitrapyrin |
| B-119 | one individualized compound I | Nuarimol |
| B-120 | one individualized compound I | Pyrimethanil |
| B-121 | one individualized compound I | Triforine |
| B-122 | one individualized compound I | Fenpiclonil |
| B-123 | one individualized compound I | Fludioxonil |
| B-124 | one individualized compound I | Aldimorph |
| B-125 | one individualized compound I | Dodemorph |
| B-126 | one individualized compound I | Dodemorph-acetate |
| B-127 | one individualized compound I | Fenpropimorph |
| B-128 | one individualized compound I | Tridemorph |
| B-129 | one individualized compound I | Fenpropidin |
| B-130 | one individualized compound I | Fluoroimid |
| B-131 | one individualized compound I | Iprodione |
| B-132 | one individualized compound I | Procymidone |
| B-133 | one individualized compound I | Vinclozolin |
| B-134 | one individualized compound I | Famoxadone |
| B-135 | one individualized compound I | Fenamidone |
| B-136 | one individualized compound I | Flutianil |
| B-137 | one individualized compound I | Octhilinone |
| B-138 | one individualized compound I | Probenazole |
| B-139 | one individualized compound I | 5-Amino-2-iso-propyl-4-ortho-tolyl-2,3- |
| | | dihydro-pyrazole-1-carbothioic acid S- |
| | | allyl ester |
| B-140 | one individualized compound I | Acibenzolar-S-methyl |
| B-141 | one individualized compound I | Amisulbrom |
| B-142 | one individualized compound I | Anilazin |
| B-143 | one individualized compound I | Blasticidin-S |
| B-144 | one individualized compound I | Captafol |
| B-145 | one individualized compound I | Captan |
| B-146 | one individualized compound I | Chinomethionat |
| B-147 | one individualized compound I | Dazomet |
| B-148 | one individualized compound I | Debacarb |
| B-149 | one individualized compound I | Diclomezine |
| B-150 | one individualized compound I | Difenzoquat, |
| B-151 | one individualized compound I | Difenzoquat-methylsulfate |
| B-152 | one individualized compound I | Fenoxanil |
| B-153 | one individualized compound I | Folpet |
| B-154 | one individualized compound I | Oxolinsäure |
| B-155 | one individualized compound I | Piperalin |
| B-156 | one individualized compound I | Proquinazid |
| B-157 | one individualized compound I | Pyroquilon |
| B-158 | one individualized compound I | Quinoxyfen |
| B-159 | one individualized compound I | Triazoxid |
| B-160 | one individualized compound I | Tricyclazole |
| B-161 | one individualized compound I | 2-Butoxy-6-iodo-3-propyl-chromen-4-one |
| B-162 | one individualized compound I | 5-Chloro-1-(4,6-dimethoxy-pyrimidin-2- |
| | | yl)-2-methyl-1H-benzoimidazole |
| B-163 | one individualized compound I | 5-Chloro-7-(4-methyl-piperidin-1-yl)- |
| | | 6-(2,4,6-trifluoro-phenyl)-[1,2,4]tri- |
| | | azolo[1,5-a]pyrimidine |
| B-164 | one individualized compound I | 5-ethyl-6-octyl-[1,2,4]triazolo[1,5- |
| | | a]pyrimidine-7-ylamine |
| B-165 | one individualized compound I | Ferbam |
| B-166 | one individualized compound I | Mancozeb |
| B-167 | one individualized compound I | Maneb |
| B-168 | one individualized compound I | Metam |
| B-169 | one individualized compound I | Methasulphocarb |
| B-170 | one individualized compound I | Metiram |
| B-171 | one individualized compound I | Propineb |
| B-172 | one individualized compound I | Thiram |
| B-173 | one individualized compound I | Zineb |
| B-174 | one individualized compound I | Ziram |
| B-175 | one individualized compound I | Diethofencarb |
| B-176 | one individualized compound I | Benthiavalicarb |
| B-177 | one individualized compound I | Iprovalicarb |
| B-178 | one individualized compound I | Propamocarb |
| B-179 | one individualized compound I | Propamocarb hydrochlorid |
| B-180 | one individualized compound I | Valiphenal |
| B-181 | one individualized compound I | N-(1-(1-(4- |
| | | cyanophenyl)ethanesulfonyl)-but-2-yl) |
| | | carbamic acid-(4-fluorophenyl) ester |
| B-182 | one individualized compound I | Dodine |
| B-183 | one individualized compound I | Dodine free base |
| B-184 | one individualized compound I | Guazatine |
| B-185 | one individualized compound I | Guazatine-acetate |
| B-186 | one individualized compound I | Iminoctadine |
| B-187 | one individualized compound I | Iminoctadine-triacetate |
| B-188 | one individualized compound I | Iminoctadine-tris(albesilate) |
| B-189 | one individualized compound I | Kasugamycin |
| B-190 | one individualized compound I | Kasugamycin-hydrochloride-hydrate |
| B-191 | one individualized compound I | Polyoxine |
| B-192 | one individualized compound I | Streptomycin |
| B-193 | one individualized compound I | Validamycin A |
| B-194 | one individualized compound I | Binapacryl |
| B-195 | one individualized compound I | Dicloran |
| B-196 | one individualized compound I | Dinobuton |
| B-197 | one individualized compound I | Dinocap |
| B-198 | one individualized compound I | Nitrothal-isopropyl |
| B-199 | one individualized compound I | Tecnazen |
| B-200 | one individualized compound I | Fentin salts |
| B-201 | one individualized compound I | Dithianon |
| B-202 | one individualized compound I | Isoprothiolane |
| B-203 | one individualized compound I | Edifenphos |
| B-204 | one individualized compound I | Fosetyl, Fosetyl-aluminium |
| B-205 | one individualized compound I | Iprobenfos |
| B-206 | one individualized compound I | Phosphorous acid (H₃PO₃) and derivatives |
| B-207 | one individualized compound I | Pyrazophos |
| B-208 | one individualized compound I | Tolclofos-methyl |
| B-209 | one individualized compound I | Chlorothalonil |
| B-210 | one individualized compound I | Dichlofluanid |
| B-211 | one individualized compound I | Dichlorophen |
| B-212 | one individualized compound I | Flusulfamide |
| B-213 | one individualized compound I | Hexachlorbenzene |
| B-214 | one individualized compound I | Pencycuron |
| B-215 | one individualized compound I | Pentachlorophenol and salts |
| B-216 | one individualized compound I | Phthalide |
| B-217 | one individualized compound I | Quintozene |
| B-218 | one individualized compound I | Thiophanate Methyl |
| B-219 | one individualized compound I | Tolylfluanid |
| B-220 | one individualized compound I | N-(4-chloro-2-nitro-phenyl)-N-ethyl- |
| | | 4-methyl-benzenesulfonamide |
| B-221 | one individualized compound I | Bordeaux mixture |
| B-222 | one individualized compound I | Copper acetate |
| B-223 | one individualized compound I | Copper hydroxide |
| B-224 | one individualized compound I | Copper oxychloride |
| B-225 | one individualized compound I | basic Copper sulfate |
| B-226 | one individualized compound I | Sulfur |
| B-227 | one individualized compound I | Biphenyl |
| B-228 | one individualized compound I | Bronopol |
| B-229 | one individualized compound I | Cyflufenamid |
| B-230 | one individualized compound I | Cymoxanil |
| B-231 | one individualized compound I | Diphenylamin |
| B-232 | one individualized compound I | Metrafenone |
| B-233 | one individualized compound I | Mildiomycin |
| B-234 | one individualized compound I | Oxin-copper |
| B-235 | one individualized compound I | Prohexadione calcium |
| B-236 | one individualized compound I | Spiroxamine |
| B-237 | one individualized compound I | Tolylfluanid |
| B-238 | one individualized compound I | N-(Cyclopropylmethoxyimino-(6- |
| | | difluoromethoxy-2,3-difluoro-phenyl)- |
| | | methyl)-2-phenyl acetamide |
| B-239 | one individualized compound I | N′-(4-(4-chloro-3-trifluoromethyl- |
| | | phenoxy)-2,5-dimethyl-phenyl)-N-ethyl- |
| | | N-methyl formamidine |
| B-240 | one individualized compound I | N′-(4-(4-fluoro-3-trifluoromethyl- |
| | | phenoxy)-2,5-dimethyl-phenyl)-N-ethyl- |
| | | N-methyl formamidine |
| B-241 | one individualized compound I | N′-(2-methyl-5-trifluoromethyl-4-(3-tri- |
| | | methylsilanyl-propoxy)-phenyl)-N-ethyl- |
| | | N-methyl formamidine |
| B-242 | one individualized compound I | N′-(5-difluoromethyl-2-methyl-4-(3-tri- |
| | | methylsilanyl-propoxy)-phenyl)-N-ethyl- |
| | | N-methyl formamidine |
| B-243 | one individualized compound I | 2-{1-[2-(5-Methyl-3-trifluoromethyl- |
| | | pyrazole-1-yl)-acetyl]-piperidin-4-yl}- |
| | | thiazole-4-carboxylic acid methyl- |
| | | (1,2,3,4-tetrahydro-naphthalen-1-yl)- |
| | | amide |
| B-244 | one individualized compound I | 2-{1-[2-(5-Methyl-3-trifluoromethyl- |
| | | pyrazole-1-yl)-acetyl]-piperidin-4-yl}- |
| | | thiazole-4-carboxylic acid methyl-(R)- |
| | | 1,2,3,4-tetrahydro-naphthalen-1-yl- |
| | | amide |
| B-245 | one individualized compound I | Acetic acid 6-tert.-butyl-8-fluoro-2,3- |
| | | dimethyl-quinolin-4-yl ester |
| B-246 | one individualized compound I | Methoxy-acetic acid 6-tert-butyl-8- |
| | | fluoro-2,3-dimethyl-quinolin-4-yl ester |
| B-247 | one individualized compound I | Carbaryl |
| B-248 | one individualized compound I | Carbofuran |
| B-249 | one individualized compound I | Carbosulfan |
| B-250 | one individualized compound I | Methomylthiodicarb |
| B-251 | one individualized compound I | Bifenthrin |
| B-252 | one individualized compound I | Cyfluthrin |
| B-253 | one individualized compound I | Cypermethrin |
| B-254 | one individualized compound I | alpha-Cypermethrin |
| B-255 | one individualized compound I | zeta-Cypermethrin |
| B-256 | one individualized compound I | Deltamethrin |
| B-257 | one individualized compound I | Esfenvalerate |
| B-258 | one individualized compound I | Lambda-cyhalothrin |
| B-259 | one individualized compound I | Permethrin |
| B-260 | one individualized compound I | Tefluthrin |
| B-261 | one individualized compound I | Diflubenzuron |
| B-262 | one individualized compound I | Flufenoxuron |
| B-263 | one individualized compound I | Lufenuron |
| B-264 | one individualized compound I | Teflubenzuron |
| B-265 | one individualized compound I | Spirotetramate |
| B-266 | one individualized compound I | Clothianidin |
| B-267 | one individualized compound I | Dinotefuran |
| B-268 | one individualized compound I | Imidacloprid |
| B-269 | one individualized compound I | Thiamethoxam |
| B-270 | one individualized compound I | Acetamiprid |
| B-271 | one individualized compound I | Thiacloprid |
| B-272 | one individualized compound I | Endosulfan |
| B-273 | one individualized compound I | Fipronil |
| B-274 | one individualized compound I | Abamectin |
| B-275 | one individualized compound I | Emamectin |
| B-276 | one individualized compound I | Spinosad |
| B-277 | one individualized compound I | Spinetoram |
| B-278 | one individualized compound I | Hydramethylnon |
| B-279 | one individualized compound I | Chlorfenapyr |
| B-280 | one individualized compound I | Fenbutatin oxide |
| B-281 | one individualized compound I | Indoxacarb |
| B-282 | one individualized compound I | Metaflumizone |
| B-283 | one individualized compound I | Flonicamid |
| B-284 | one individualized compound I | Lubendiamide |
| B-285 | one individualized compound I | Chlorantraniliprole |
| B-286 | one individualized compound I | Cyazypyr (HGW86) |
| B-287 | one individualized compound I | Cyflumetofen |
| B-288 | one individualized compound I | Acetochlor |
| B-289 | one individualized compound I | Dimethenamid |
| B-290 | one individualized compound I | metolachlor |
| B-291 | one individualized compound I | Metazachlor |
| B-292 | one individualized compound I | Glyphosate |
| B-293 | one individualized compound I | Glufosinate |
| B-294 | one individualized compound I | Sulfosate |
| B-295 | one individualized compound I | Clodinafop |
| B-296 | one individualized compound I | Fenoxaprop |
| B-297 | one individualized compound I | Fluazifop |
| B-298 | one individualized compound I | Haloxyfop |
| B-299 | one individualized compound I | Paraquat |
| B-300 | one individualized compound I | Phenmedipham |
| B-301 | one individualized compound I | Clethodim |
| B-302 | one individualized compound I | Cycloxydim |
| B-303 | one individualized compound I | Profoxydim |
| B-304 | one individualized compound I | Sethoxydim |
| B-305 | one individualized compound I | Tepraloxydim |
| B-306 | one individualized compound I | Pendimethalin |
| B-307 | one individualized compound I | Prodiamine |
| B-308 | one individualized compound I | Trifluralin |
| B-309 | one individualized compound I | Acifluorfen |
| B-310 | one individualized compound I | Bromoxynil |
| B-311 | one individualized compound I | Imazamethabenz |
| B-312 | one individualized compound I | Imazamox |
| B-313 | one individualized compound I | Imazapic |
| B-314 | one individualized compound I | Imazapyr |
| B-315 | one individualized compound I | Imazaquin |
| B-316 | one individualized compound I | Imazethapyr |
| B-317 | one individualized compound I | 2,4-Dichlorophenoxyacetic acid (2,4-D) |
| B-318 | one individualized compound I | Chloridazon |
| B-319 | one individualized compound I | Clopyralid |
| B-320 | one individualized compound I | Fluroxypyr |
| B-321 | one individualized compound I | Picloram |
| B-322 | one individualized compound I | Picolinafen |
| B-323 | one individualized compound I | Bensulfuron |
| B-324 | one individualized compound I | Chlorimuron-ethyl |
| B-325 | one individualized compound I | Cyclosulfamuron |
| B-326 | one individualized compound I | Iodosulfuron |
| B-327 | one individualized compound I | Mesosulfuron |
| B-328 | one individualized compound I | Metsulfuron-methyl |
| B-329 | one individualized compound I | Nicosulfuron |
| B-330 | one individualized compound I | Rimsulfuron |
| B-331 | one individualized compound I | Triflusulfuron |
| B-332 | one individualized compound I | Atrazine |
| B-333 | one individualized compound I | Hexazinone |
| B-334 | one individualized compound I | Diuron |
| B-335 | one individualized compound I | Florasulam |
| B-336 | one individualized compound I | Pyroxasulfone |
| B-337 | one individualized compound I | Bentazone |
| B-338 | one individualized compound I | Cinidon-ethlyl |
| B-339 | one individualized compound I | Cinmethylin |
| B-340 | one individualized compound I | Dicamba |
| B-341 | one individualized compound I | Diflufenzopyr |
| B-342 | one individualized compound I | Quinclorac |
| B-343 | one individualized compound I | Quinmerac |
| B-344 | one individualized compound I | Mesotrione |
| B-345 | one individualized compound I | Saflufenacil |
| B-346 | one individualized compound I | Topramezone |

The active substances referred to as component 2, their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The mixtures of active substances can be prepared as compositions comprising besides the active ingridients at least one inert ingredient by usual means, e. g. by the means given for the compositions of compounds I.

Concerning usual ingredients of such compositions reference is made to the explanations given for the compositions containing compounds I.

The mixtures of active substances according to the present invention are suitable as fungicides, as are the compounds of formula I. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is referred to the explanations regarding the fungicidal activity of the compounds and the compositions containing compounds I, respectively.

### SYNTHESIS EXAMPLES

With due modification of the starting compounds, the procedures shown in the synthesis examples below were used to obtain further compounds I. The resulting compounds I, together with physical data, are listed in Tables 1-a and 1-b below.

### I. Preparation of Intermediates

### I.1 Preparation of Compounds II

### Example 1

### Preparation of C-(2-methoxy-pyrimidin-4-yl)-methyl amine

### 1a) Preparation of 2-methoxy-4-methyl-pyrimidine

4,4-Dimethoxy-butan-1-one (26.4 g) and O-methyl isourea (33.2 g) were refluxed in sodium methoxide (30%) for 3 days. The solvent was removed in vacuo. After distillation, 16 g of the title compound were obtained.

1H-NMR (CDCl₃, TMS): δ=2.50 (s, 3H, Me), 4.00 (s, 3H, OMe), 6.80 (1H), 8.35 (1H).

### 1b) Preparation of 2-methoxy-pyrimidine-4-carbaldehyde oxime

2-Methoxy-4-methyl pyrimidine (8.9 g) was dissolved in DMF (20 ml) and cooled to about −40° C. After addition of n-butyl nitrite (7.7 g), potassium methoxide (5.6 g) was added in small portions keeping the temperature at about −40° C. After stirring for 1 h at −40° C., the reaction mixture was warmed to about 20 to 25° C. After further stirring for 1 h, HCl (10%, 50 ml) was added. The mixture as extracted with MTBE and dried and the solvent was removed in vacuo. The title compound (6.0 g) was obtained as a light-brown solid. 1H-NMR (CDCl₃, TMS): δ=3.90 (s, 3H, OMe), 7.40 (1H), 6.80 (1H), 7.95 (1H), 8.60 (1H), 12.30 (1H). HPLC-MS: 1.18 min (M+).

### 1c) Preparation of C-(2-methoxy-pyrimidin-4-yl)-methyl amine

2-Methoxy-pyrimidine-4-carbaldehyde oxime (6.0 g) and triethylamine (3 ml) were dissolved in methanol (20 ml). The flask was evaporated and backfilled with nitrogen. Pd/C (10%, 2 g) was added and the flask was evaporated again and backfilled with hydrogen. The mixture was incubated under a hydrogen atmosphere that was established at ambient pressure for about 4 h at about 20 to 25° C. After purging with nitrogen, the reaction mixture was filtered over a plug of silica. After removing in vacuo the solvent from the resulting filtrate, the title compound (5.6 g) was obtained as a light brown solid, that solidified upon standing.

### I.2 Preparation of Compounds III

### Example 2

### Preparation of 4-(3-chloro-5-trifluormethyl-pyridin-2-yloxy)-3-methyl-benzenesulfonyl chloride Via Direct Sulfochlorination

### 2a) Preparation of 3-chloro-5-trifluormethyl-2-o-tolyloxy-pyridine

A mixture of 2,3-dichloro-5-trifluoromethylpyridine (5.0 g), o-cresol (2.5 g), potassium iodide (0.4 g) and K₂CO₃ (3.5 g) dissolved in DMF was stirred for 2 h at about 100° C. The resulting reaction mixture was added to water (50 ml) and extracted with DCM. After washing with brine, the combined organic phases were dried and the solvent was removed in vacuo. The title compound (5.7 g) was obtained as a brown oil and directly submitted to the next reaction. HPLC-MS: 4.01 min [288, M+].

### 2b) Preparation of 4-(3-chloro-5-trifluormethyl-pyridin-2-yloxy)-3-methyl-benzene-sulfonyl chloride

3-Chloro-5-trifluormethyl-2-o-tolyloxy-pyridine (1.0 g) in 1,2-dichloro-ethane (15 ml) was added dropwise to chlorosulfonic acid (1.6 ml) in 1,2-dichloro-ethane (15 ml) at 0° C. with stirring. The reaction mixture was heated to 50° C. for 14 h and cooled to 20 to 25° C., then added to 100 ml of water. The pH was adjusted with NaOH (50%) to about 14 and the mixture was extracted with MTBE. After washing with brine, the combined organic phases were dried and the solvent was removed in vacuo. The title compound (0.6 g) was obtained as a light-brown solid and directly submitted to the next reaction.

HPLC-MS: 4.01 min [386, M+].

In analogy to the abovementioned example, the following sulfochlorides were prepared: 4-(5-trifluormethyl-pyridin-2-yloxy)-3-methyl-benzenesulfonyl chloride, 4-(3-chloro-5-trifluormethyl-pyridin-2-yloxy)-2-methyl-benzenesulfonyl chloride, 4-(5-trifluormethyl-pyridin-2-yloxy)-2-methyl-benzenesulfonyl chloride, 4-(3-chloro-5-trifluormethyl-pyridin-2-yloxy)-2,3-dimethyl-benzenesulfonyl chloride, 4-(5-trifluormethyl-pyridin-2-yloxy)-2,3-dimethyl-benzenesulfonyl chloride, 4-(3-chloro-5-trifluormethyl-pyridin-2-yloxy)-2,5-dimethyl-benzenesulfonyl chloride, 4-(5-trifluormethyl-pyridin-2-yloxy)-2,5-dimethyl-benzenesulfonyl chloride, 4-(3-chloro-5-trifluormethyl-pyridin-2-yloxy)-3,5-dimethyl-benzenesulfonyl chloride, 4-(5-trifluormethyl-pyridin-2-yloxy)-3,5-dimethyl-benzenesulfonyl chloride, 4-(3-chloro-5-trifluormethyl-pyridin-2-yloxy)-2,6-dimethyl-benzenesulfonyl chloride, 4-(5-trifluormethyl-pyridin-2-yloxy)-2,6-dimethyl-benzenesulfonyl chloride.

### Example 3

### Preparation of 3-chloro-2-(2-fluoro-4-nitro-phenoxy)-5-trifluoromethyl-pyridine

### 3a) Preparation of 3-chloro-2-(2-fluoro-4-nitro-phenoxy)-5-trifluoromethyl-pyridine

A mixture of 2,3-dichloro-5-trifluoromethylpyridine (7.5 g), 2-fluoro-4-nitrophenol (6.0 g) and K₂CO₃ (7.2 g) in NMP (110 ml) was incubated for about 12 to 16 h at about 100° C. The mixture was added to water (150 ml) and extracted with MTBE. After washing with brine, the combined organic phases were dried and the solvent was removed in vacuo. The crude product was purified by means of column chromatography over SiO₂ eluting with cyclohexane/ethyl acetate (10:1) mixtures. The title compound (6.0 g) was obtained as a brown oil and directly submitted to the next reaction.

HPLC-MS: 3.91 min [337, M+H+].

### 3b) Preparation of 4-(3-chloro-5-trifluoromethyl-pyridin-2-yloxy)-3-fluoro-phenylamine

3-Chloro-2-(2-fluoro-4-nitro-phenoxy)-5-trifluoromethylpyridine (6.0 g) was dissolved in methanol (36 ml) and Raney Nickel (2.0 g, washed with MeOH) was added. After flushing with nitrogen gas, the flask was evaporated and afterwards purged with hydrogen. After hydrogenation at ambient pressure for 2 h, the reaction mixture was filtered over celite and the solvent was removed in vacuo. The title compound (3.3 g) was obtained as a colorless oil and directly submitted to the next reaction.

HPLC-MS: 3.98 min [308, M+H+].

### 3c) Preparation of 4-(3-chloro-5-trifluoromethyl-pyridin-2-yloxy)-3-fluoro-benzene-sulfonylchloride

Glacial acetic acid (10 ml) and HCl (6.6 ml) were added to 4-(3-chloro-5-trifluoromethyl-pyridin-2-yloxy)-3-fluoro-phenylamine dissolved in acetontrile (76 ml) at about 0° C. After stirring for 30 minutes, NaNO₂ dissolved in H₂O (0.9 g in 3 ml) was added slowly keeping the temperature below 5° C. After further 30 minutes of stirring at about 0° C., SO₂ (33 g) was added keeping the temperature below 5° C. After adding CuCl₂ (1.8 g) dissolved in 1 ml H₂O, the reaction mixture was stirred for further 16 h. The solvent was removed in vacuo. The mixture was added to water (200 ml) and extracted with DCM. After washing with HCl (10%), the combined organic phases were dried and the solvent was removed in vacuo. The title compound (2.9 g) was a brown oil. HPLC-MS: 4.01 min [391, M+H+].

In analogy to the abovementioned example, the following sulfonylchlorides were prepared: 4-(5-trifluoromethyl-pyridin-2-yloxy)-3-fluoro-benzenesulfonylchloride, 4-(3-chloro-5-trifluoromethyl-pyridin-2-yloxy)-2-fluoro-benzenesulfonylchloride, 4-(5-trifluoromethyl-pyridin-2-yloxy)-2-fluoro-benzenesulfonylchloride, 4-(3-chloro-5-trifluoromethyl-pyridin-2-yloxy)-3-chloro-benzenesulfonylchloride, 4-(5-trifluoromethyl-pyridin-2-yloxy)-3-chloro-benzenesulfonylchloride, 4-(3-chloro-5-trifluoromethyl-pyridin-2-yloxy)-2-chloro-benzenesulfonylchloride, 4-(5-trifluoromethyl-pyridin-2-yloxy)-2-chloro-benzenesulfonylchloride, 4-(1-methyl-5-trifluoromethyl-1H-pyrazol-3-yloxy)-benzenesulfonyl chloride, 4-(1-methyl-3-chloro-5-trifluoromethyl-1H-pyrazol-3-yloxy)-benzenesulfonyl chloride, 4-(3-chloro-5-trifluoromethyl-pyridin-2-yloxy)-2-trifluoromethyl benzene-sulfonylchloride, 4-(5-trifluoromethyl-pyridin-2-yloxy)-2-trifluoromethyl-benzenesulfonylchloride, 4-(3-chloro-5-trifluoromethyl-pyridin-2-yloxy)-3-trifluoromethyl benzene-sulfonylchloride, 4-(5-trifluoromethyl-pyridin-2-yloxy)-3-trifluoromethyl-benzenesulfonylchloride.

### II. Preparation of Compounds I

### Example 4

### Preparation of 4-(3-chloro-5-trifluoromethyl-pyridin-2-yloxy)-N-(2-methoxy-pyrimidin-4-ylmethyl)-3-methyl-benzenesulfonamide (Table I: Example No. 1-17)

4-(3-Chloro-5-trifluormethyl-pyridin-2-yloxy)-3-methyl-benzenesulfonyl chloride (277 mg) in DCM (2 ml) was added slowly to a solution of (2-methoxy-pyrimidin-4-yl)-methylamine (100 mg) and N,N′-diisopropylethylamine (0.3 ml) in DCM (2 ml) at 0° C. After stirring for about 16 to 20 h at 20 to 25° C., the solvent was removed in vacuo. The residue was purified by means of column chromatography over SiO₂ eluting with cyclohexane/ethyl acetate (1:1) mixtures. The title compound was obtained as a colorless oil. HPLC-MS: 3.46 min [489, M+].

TABLE I-a
Compounds of formula I.A to I.K.

| ex. | | | | | | | m.p. [° C.]; |
|---|---|---|---|---|---|---|---|
| no | form.* | R^{a1} | R^{a2} | R^{a3} | A** | Het | Rₜ [min] |
| I-1 | I.A | OCH₃ | H | H | A-1 | 3-chloro-5-trifluoromethyl- | 137-139° C. |
| | | | | | | pyridin-2-yl | |
| I-2 | I.A | SCHF₂ | H | H | A-1 | 3-chloro-5-trifluoromethyl- | 3.89 min |
| | | | | | | pyridin-2-yl | |
| I-3 | I.A | SCF₃ | H | H | A-1 | 3-chloro-5-trifluoromethyl- | 4.05 min |
| | | | | | | pyridin-2-yl | |
| I-4 | I.A | H | H | H | A-1 | 3-chloro-5-trifluoromethyl- | 3.15 min |
| | | | | | | pyridin-2-yl | |
| I-5 | I.A | SCH₃ | H | H | A-1 | 3-chloro-5-trifluoromethyl- | 3.66 min |
| | | | | | | pyridin-2-yl | |
| I-6 | I.A | OCHF₂ | H | H | A-1 | 3-chloro-5-trifluoromethyl- | 3.67 min |
| | | | | | | pyridin-2-yl | |
| I-7 | I.A | OCH₃ | H | H | A-1 | 5-bromopyridin-2-yl | 2.97 min |
| I-8 | I.A | OCH₃ | H | H | A-1 | 5-chloropyridin-2-yl | 127° C. |
| I-9 | I.A | OCH₃ | H | H | A-1 | 3,5-dichloropyridin-2-yl | 3.24 min |
| I-10 | I.A | OCH₃ | H | H | A-1 | 3-trifluoromethyl-pyridin-2-yl | 3.03 min |
| I-11 | I.A | OCH₃ | H | H | A-1 | 4-trifluoromethyl-pyridin-2-yl | 107° C. |
| I-12 | I.A | OCH₃ | H | H | A-1 | 3-trifluoromethyl-5- | 3.37 min |
| | | | | | | chloropyridin-2-yl | |
| I-13 | I.A | OCH₃ | H | H | A-1 | 3-methyl-5-trifluoromethyl- | 3.34 min |
| | | | | | | pyridin-2-yl | |
| I-14 | I.A | OCH₃ | H | H | A-1 | 3-fluoro-5-trifluoromethyl- | 3.22 min |
| | | | | | | pyridin-2-yl | |
| I-15 | I.A | OCH₃ | H | H | A-1 | 3-chloropyridin-2-yl | 2.83 min |
| I-16 | I.A | OCH₃ | H | H | A-1 | 6-trifluoromethyl-pyridin-2-yl | 3.06 min |
| I-17 | I.A | OCH₃ | H | H | A-2 | 3-chloro-5-trifluoromethyl- | 138° C. |
| | | | | | | pyridin-2-yl | |
| I-18 | I.A | OCH₃ | H | H | A-3 | 3-chloro-5-trifluoromethyl- | 97° C.; |
| | | | | | | pyridin-2-yl | 3.54 min |
| I-19 | I.A | OCH₃ | H | H | A-2 | 3-trifluoromethyl-pyridin-2-yl | 3.19 min |
| I-20 | I.A | OCH₃ | H | H | A-3 | 3-trifluoromethyl-pyridin-2-yl | 3.18 min |
| I-21 | I.A | OCH₃ | H | H | A-2 | 5-trifluoromethyl-pyridin-2-yl | 3.24 min |
| I-22 | I.A | OCH₃ | H | H | A-3 | 5-trifluoromethyl-pyridin-2-yl | 3.25 min |
| I-23 | I.A | OCH₃ | H | H | A-1 | 5-trifluoromethyl-6-chloro- | 120° C. |
| | | | | | | pyridin-2-yl | |
| I-24 | I.A | OCH₃ | H | H | A-1 | 3,6-dichloro-5-trifluoro- | 141° C. |
| | | | | | | methyl-pyridin-2-yl | |
| I-25 | I.A | cyclo- | H | H | A-1 | 3-chloro-5-trifluoromethyl- | 132° C. |
| | | propyl | | | | pyridin-2-yl | |
| I-26 | I.A | cyclo- | H | H | A-1 | 5-trifluoromethyl-pyridin-2-yl | 3.15 min |
| | | propyl | | | | | |
| I-27 | I.A | cyclo- | H | H | A-1 | 3-trifluoromethyl-pyridin-2-yl | 3.08 min |
| | | propyl | | | | | |
| I-28 | I.A | H | H | CH₃ | A-1 | 3-chloro-5-trifluoromethyl- | 92° C. |
| | | | | | | pyridin-2-yl | |
| I-29 | I.A | H | H | CH₃ | A-1 | 5-trifluoromethyl-pyridin-2-yl | 2.88 min |
| I-30 | I.A | cyclo- | H | CH₃ | A-1 | 3-trifluoromethyl-pyridin-2-yl | 2.81 min |
| | | propyl | | | | | |
| I-31 | I.A | H | CH₃ | CH₃ | A-1 | 3-chloro-5-trifluoromethyl- | 187° C. |
| | | | | | | pyridin-2-yl | |
| I-32 | I.A | H | CH₃ | CH₃ | A-1 | 5-trifluoromethyl-pyridin-2-yl | 114° C. |
| I-33 | I.A | H | CH₃ | CH₃ | A-1 | 3-trifluoromethyl-pyridin-2-yl | 120° C. |
| I-34 | I.A | CH₃ | CH₃ | CH₃ | A-1 | 3-chloro-5-trifluoromethyl- | 2.97 min |
| | | | | | | pyridin-2-yl | |
| I-35 | I.A | CH₃ | CH₃ | CH₃ | A-1 | 5-trifluoromethyl-pyridin-2-yl | 88° C. |
| I-36 | I.A | CH₃ | CH₃ | CH₃ | A-1 | 3-trifluoromethyl-pyridin-2-yl | 101° C. |
| I-37 | I.A | OCH₃ | H | CH₃ | A-1 | 3-chloro-5-trifluoromethyl- | 114° C. |
| | | | | | | pyridin-2-yl | |
| I-38 | I.A | OCH₃ | H | CH₃ | A-1 | 5-trifluoromethyl-pyridin-2-yl | 127° C. |
| I-39 | I.A | OCH₃ | H | CH₃ | A-1 | 3-trifluoromethyl-pyridin-2-yl | 128° C. |
| I-40 | I.A | H | H | OCH₃ | A-1 | 3-chloro-5-trifluoromethyl- | 119° C. |
| | | | | | | pyridin-2-yl | |
| I-41 | I.A | H | H | OCH₃ | A-1 | 5-trifluoromethyl-pyridin-2-yl | 117° C. |
| I-42 | I.A | H | H | OCH₃ | A-1 | 3-trifluoromethyl-pyridin-2-yl | 124° C. |
| I-43 | I.A | CH₃ | H | H | A-1 | 3-chloro-5-trifluoromethyl- | 121° C. |
| | | | | | | pyridin-2-yl | |
| I-44 | I.A | CH₃ | H | H | A-1 | 5-trifluoromethyl-pyridin-2-yl | 114° C. |
| I-45 | I.A | CH₃ | H | H | A-1 | 3-trifluoromethyl-pyridin-2-yl | 88° C. |
| I-46 | I.A | OCH₃ | H | H | A-1 | 4-trifluoromethyl-6-methyl- | 140° C. |
| | | | | | | pyridin-2-yl | |
| I-47 | I.A | OCH₃ | H | H | A-1 | 2-trifluoromethyl-pyrimidin-4- | 144° C. |
| | | | | | | yl | |
| I-48 | I.A | OCH₃ | H | H | A-1 | 2-trifluoromethyl-5,6-di- | 143° C. |
| | | | | | | methylpyrimidin-4-yl | |
| I-49 | I.A | OCH₃ | H | H | A-1 | 3-chloro-4-methyl-5-trifluoro- | 137° C. |
| | | | | | | methyl-pyridin-2-yl | |
| I-50 | I.A | OCH₃ | H | H | A-1 | 4-methyl-5-trifluoromethyl- | 145° C. |
| | | | | | | pyridin-2-yl | |
| I-51 | I.A | OCH₂CH₃ | H | H | A-1 | 3-chloro-5-trifluoromethyl- | 111° C. |
| | | | | | | pyridin-2-yl | |
| I-52 | I.A | OCH₂CH₃ | H | H | A-1 | 3-trifluoromethyl-pyridin-2-yl | 3.21 min |
| I-53 | I.A | OCH₂CH₃ | H | H | A-1 | 5-trifluoromethyl-pyridin-2-yl | 110° C. |
| I-54 | I.A | ethoxy | H | H | A-1 | 3-chloro-5-trifluoromethyl- | 3.70 min |
| | | | | | | pyridin-2-yl | |
| I-55 | I.A | ethoxy | H | H | A-1 | 3-trifluoromethyl-pyridin-2-yl | 146° C. |
| I-56 | I.A | ethoxy | H | H | A-1 | 5-trifluoromethyl-pyridin-2-yl | 133° C. |
| I-57 | I.A | OCH₂CF₃ | H | H | A-1 | 3-chloro-5-trifluoromethyl- | 129° C. |
| | | | | | | pyridin-2-yl | |
| I-58 | I.A | OCH₃ | F | H | A-1 | 5-trifluoromethyl-pyridin-2-yl | 3.27 min |
| I-59 | I.A | OCH₃ | F | H | A-1 | 3-chloro-5-trifluoromethyl- | 3.55 min |
| | | | | | | pyridin-2-yl | |
| I-60 | I.A | OCH₃ | F | H | A-1 | 3-trifluoromethyl-pyridin-2-yl | 3.20 min |
| I-61 | I.A | OCH₂CF₃ | H | H | A-1 | 3-trifluoromethyl-pyridin-2-yl | 175° C. |
| I-62 | I.A | OCH₂CF₃ | H | H | A-1 | 5-trifluoromethyl-pyridin-2-yl | 111° C. |
| I-63 | I.A | OCH₃ | H | H | A-1 | 3,5-difluoropyridin-2-yl | 129° C. |
| I-64 | I.A | OCH₃ | H | CF₃ | A-1 | 3-chloro-5-trifluoromethyl- | 142° C. |
| | | | | | | pyridin-2-yl | |
| I-65 | I.A | OCH₃ | H | CF₃ | A-1 | 3-trifluoromethyl-pyridin-2-yl | 3.59 min |
| I-66 | I.A | OCH₃ | H | CF₃ | A-1 | 5-trifluoromethyl-pyridin-2-yl | 3.67 min |
| I-67 | I.A | OCH₃ | CH₃ | CH₃ | A-1 | 3-chloro-5-trifluoromethyl- | 123° C. |
| | | | | | | pyridin-2-yl | |
| I-68 | I.A | OCH₃ | CH₃ | CH₃ | A-1 | 3-trifluoromethyl-pyridin-2-yl | 3.20 min |
| I-69 | I.A | OCH₃ | CH₃ | CH₃ | A-1 | 5-trifluoromethyl-pyridin-2-yl | 3.27 min |
| I-70 | I.A | SCH₃ | F | H | A-1 | 3-chloro-5-trifluoromethyl- | 3.78 min |
| | | | | | | pyridin-2-yl | |
| I-71 | I.A | SCH₃ | F | H | A-1 | 3-trifluoromethyl-pyridin-2-yl | 3.45 min |
| I-72 | I.A | SCH₃ | F | H | A-1 | 5-trifluoromethyl-pyridin-2-yl | 3.42 min |
| I-73 | I.A | OCH₃ | H | H | A- | 3-chloro-5-trifluoromethyl- | 174° C. |
| | | | | | 20 | pyridin-2-yl | |
| I-74 | I.A | OCH₃ | H | H | A- | 3-chloro-5-trifluoromethyl- | 3.45 min |
| | | | | | 23 | pyridin-2-yl | |
| I-75 | I.A | OCH₃ | H | H | A- | 3-chloro-5-trifluoromethyl- | 142° C. |
| | | | | | 19 | pyridin-2-yl | |
| I-76 | I.A | OCH₃ | H | H | A- | 5-trifluoromethyl-pyridin-2-yl | 138° C. |
| | | | | | 20 | | |
| I-77 | I.A | OCH₃ | H | H | A- | 5-trifluoromethyl-pyridin-2-yl | 3.24 min |
| | | | | | 19 | | |
| I-78 | I.A | OCH₃ | H | H | A- | 5-trifluoromethyl-pyridin-2-yl | 3.35 min |
| | | | | | 23 | | |
| I-79 | I.A | OCH₃ | CH₃ | H | A-1 | 5-chloro-pyridin-2-yl | 147° C. |
| I-80 | I.A | OCH₃ | CH₃ | H | A-1 | 5-trifluoromethyl-pyridin-2-yl | 3.25 min |
| I-81 | I.A | OCH₃ | H | H | A-7 | 3-chloro-5-trifluoromethyl- | 3.65 min |
| | | | | | | pyridin-2-yl | |
| I-82 | I.A | OCH₃ | H | H | A-7 | 5-trifluoromethyl-pyridin-2-yl | 3.36 min |
| I-83 | I.A | OCH₃ | H | H | A-4 | 3-chloro-5-trifluoromethyl- | 3.75 min |
| | | | | | | pyridin-2-yl | |
| I-84 | I.A | OCH₃ | H | H | A-4 | 5-trifluoromethyl-pyridin-2-yl | 3.45 min |
| I-85 | I.A | OCH₃ | H | H | A-5 | 3-chloro-5-trifluoromethyl- | 3.41 min |
| | | | | | | pyridin-2-yl | |
| I-86 | I.A | OCH₃ | H | H | A-5 | 5-trifluoromethyl-pyridin-2-yl | 3.45 min |
| I-87 | I.A | OCH₃ | H | H | A-1 | 5-(1-methoxyimino-ethyl)- | 3.00 min |
| | | | | | | pyridin-2-yl | |
| I-88 | I.A | OCH₃ | H | OCH₃ | A-1 | 5-trifluoromethyl-pyridin-2-yl | 111° C. |
| I-89 | I.A | OCH₃ | H | OCH₃ | A-1 | 3-chloro-5-trifluoromethyl- | 127° C. |
| | | | | | | pyridin-2-yl | |
| I-90 | I.A | H | OCH₃ | H | A-1 | 5-trifluoromethyl-pyridin-2-yl | 121° C. |
| I-91 | I.A | H | OCH₃ | H | A-1 | 3-chloro-5-trifluoromethyl- | 113° C. |
| | | | | | | pyridin-2-yl | |
| I-92 | I.A | CH₃ | H | OCH₃ | A-1 | 3-chloro-5-trifluoromethyl- | 149° C. |
| | | | | | | pyridin-2-yl | |
| I-93 | I.A | CH₃ | H | OCH₃ | A-1 | 5-trifluoromethyl-pyridin-2-yl | 87-89° C. |
| I-94 | I.A | OCH₃ | H | CH₃ | A-2 | 3-chloro-5-trifluoromethyl- | 113° C. |
| | | | | | | pyridin-2-yl | |
| I-95 | I.A | OCH₃ | %-(CH₂)₂—O-# | | A-1 | 3-chloro-5-trifluoromethyl- | 176° C. |
| | | | | | | pyridin-2-yl | |
| I-96 | I.A | OCH₃ | %-(CH₂)₂—O-# | | A-2 | 3-chloro-5-trifluoromethyl- | 78° C. |
| | | | | | | pyridin-2-yl | |
| I-97 | I.A | OCH₃ | %-(CH₂)₂—O-# | | A-3 | 3-chloro-5-trifluoromethyl- | 79° C. |
| | | | | | | pyridin-2-yl | |
| I-98 | I.A | OCH₃ | %-(CH₂)₃-# | | A-1 | 3-chloro-5-trifluoromethyl- | 139-140° C. |
| | | | | | | pyridin-2-yl | |
| I-99 | I.A | OCH₃ | %-(CH₂)₃-# | | A-2 | 3-chloro-5-trifluoromethyl- | 150-152° C. |
| | | | | | | pyridin-2-yl | |
| I-100 | I.A | OCH₃ | %-(CH₂)₃-# | | A-3 | 3-chloro-5-trifluoromethyl- | 137-139° C. |
| | | | | | | pyridin-2-yl | |
| I-101 | I.A | CF₃ | H | H | A-2 | 3-chloro-5-trifluoromethyl- | 132° C.; |
| | | | | | | pyridin-2-yl | 3.59 min |
| I-102 | I.A | CF₃ | H | H | A-3 | 3-chloro-5-trifluoromethyl- | 3.79 min |
| | | | | | | pyridin-2-yl | |
| I-103 | I.A | CF₃ | H | H | A-3 | 5-trifluoromethyl-pyridin-2-yl | 3.55 min |
| I-104 | I.A | CF₃ | H | H | A-2 | 5-trifluoromethyl-pyridin-2-yl | 3.55 min |
| I-105 | I.A | CF₃ | H | H | A-1 | 3-chloro-5-trifluoromethyl- | 3.66 min |
| | | | | | | pyridin-2-yl | |
| I-106 | I.A | CF₃ | H | H | A-1 | 5-trifluoromethyl-pyridin-2-yl | 3.42 min |
| I-107 | I.A | OCH₃ | H | H | A-1 | 1-methyl-4-chloro-5-trifluoromethyl- | 138° C. |
| | | | | | | 1H-pyrazol-3-yl | |
| I-108 | I.G | OCH₃ | H | H | A-1 | 1-methyl-3-trifluoromethyl- | 142° C. |
| | | | | | | 1H-pyrazol-4-yl | |
| I-109 | I.A | OCH₃ | H | H | A-1 | 3-trifluoromethyl-pyridin-4-yl | 112° C. |
| I-110 | I.A | OCH₃ | H | H | A-1 | 6-trifluormethyl-pyridazin-3-yl | 170° C. |
| I-111 | I.A | OCH₃ | H | H | A-1 | quinolin-4-yl | 2.05 min |
| I-112 | I.G | OCH₃ | H | H | A-1 | 3-ethyl-isoxazol-5-yl | 98° C. |
| I-113 | I.G | OCH₃ | H | H | A-1 | 4-trifluoromethyl-pyridin-2-yl | 107° C. |
| I-114 | I.G | OCH₃ | H | H | A-1 | 2-methyl-4-trifluoromethyl- | 114° C. |
| | | | | | | thiazol-5-yl | |
| I-115 | I.G | OCH₃ | H | H | A-1 | 3-trifluoromethyl-pyridin-2-yl | 2.86 min |
| I-116 | I.G | OCH₃ | H | H | A-1 | 6-trifluoromethyl-pyridin-2-yl | 140° C. |
| I-117 | I.G | OCH₃ | H | H | A-1 | 2-methyl-4-chloro-5-trifluoromethyl- | 131° C. |
| | | | | | | 2H-pyrazol-3-yl | |
| I-118 | I.A | OCH₃ | H | OCH₃ | A-1 | 3-chloro-5-trifluoromethyl- | 3.59 min |
| | | | | | | pyridin-2-yl | |
| I-119 | I.A | OCH₃ | H | H | A-2 | 3,5-dichloro-pyridin-2-yl | 3.55 min |
| I-120 | I.A | OCH₃ | H | H | A-3 | 3,5-dichloro-pyridin-2-yl | 141° C. |
| I-121 | I.A | OCH₃ | H | H | A-2 | 5-chloro-pyridin-2-yl | 3.20 min |
| I-122 | I.A | OCH₃ | H | H | A-3 | 5-chloro-pyridin-2-yl | 90-93° C. |
| I-123 | I.G | OCH₃ | H | H | A-1 | pyridin-4-yl | |
| I-124 | I.G | OCH₃ | H | H | A-1 | 2-chloro-thiazol-5-yl | 130° C. |
| I-125 | I.G | OCH₃ | H | H | A-1 | 5-trifluoromethyl-pyridin-3-yl | 127° C. |
| I-126 | I.A | OCH₃ | H | H | A-1 | 3-chloro-5-ethoxycarbonyl- | 125° C. |
| | | | | | | pyridin-2-yl | |
| I-127 | I.A | OCH₃ | H | H | A-1 | 3-bromo-pyridin-4-yl | 133° C. |
| I-128 | I.J | OCH₃ | H | H | A-1 | 3-chloro-5-trifluoromethyl- | 147° C. |
| | | | | | | pyridin-2-yl | |
| I-129 | I.A | OCH₃ | H | H | A-1 | 5-methoxycarbonyl-pyridin-2- | 127° C. |
| | | | | | | yl | |
| I-130 | I.G | OCH₃ | H | H | A-1 | 2,5-dimethyl-2H-pyrazol-3-yl | 42° C. |
| I-131 | I.G | OCH₃ | H | H | A-1 | 3-(pyridin-3-yl)-isoxazol-5-yl | 51° C. |
| I-132 | I.A | OCH₃ | H | H | A-2 | 3-fluoro-5-chloro-pyridin-2-yl | 57° C. |
| I-133 | I.A | OCH₃ | H | H | A-3 | 3-fluoro-5-chloro-pyridin-2-yl | 53° C. |
| I-134 | I.A | OCH₃ | H | H | A-1 | 3-chloro-pyridin-4-yl | 2.22 min |
| I-135 | I.J | OCH₃ | H | H | A-1 | 5-trifluoromethyl-pyridin-2-yl | 122° C. |
| I-136 | I.A | OCH₃ | H | H | A-1 | 3-bromo-5-methyl-pyridin-2-yl | 174° C. |
| I-137 | I.G | OCH₃ | H | H | A-1 | 2-methyl-5-trifluoromethyl-2H- | 3.01 min |
| | | | | | | pyrazol-3-yl | |
| I-138 | I.G | OCH₃ | H | H | A-1 | thiazol-4-yl | 2.34 min |
| I-139 | I.A | OCH₃ | H | H | A- | 3-chloro-5-trifluoromethyl- | 3.86 min |
| | | | | | 22 | pyridin-2-yl | |
| I-140 | I.A | 4-F-Phenyl | H | H | A-1 | 5-trifluoromethyl-pyridin-2-yl | |
| I-141 | I.G | 4-F-Phenyl | H | H | A-1 | 2-ethyl-5-trifluoromethyl-2H- | |
| | | | | | | pyrazol-3-yl | |
| I-142 | I.G | 4-F-Phenyl | H | H | A-1 | 2,5-dimethyl-2H-pyrazol-3-yl | |
| I-143 | I.G | OCH₃ | H | H | A-1 | 2-methyl-5-cyclopropyl- | |
| | | | | | | 2H-pyrazol-3-yl | |
| I-144 | I.G | OCH₃ | H | H | A-1 | 3-cyclohexyl-isoxazol-5-yl | 133° C. |
| I-145 | I.G | OCH₃ | H | H | A-1 | 2-trifluoromethyl-thiazol-5-yl | 127° C. |
| I-146 | I.G | OCH₃ | H | H | A-1 | 3-(pyridin-4-yl)-isoxazol-5-yl | 129° C. |
| I-147 | I.G | OCH₃ | H | H | A-1 | 5-trifluoromethyl-pyridin-2-yl | 148° C. |
| I-148 | I.G | OCH₃ | H | H | A-1 | 3-methyl-isoxazol-5-yl | 114° C. |
| I-149 | I.G | OCH₃ | H | H | A-1 | benzothiazol-2-yl | 188° C. |
| I-150 | I.A | OCH₃ | H | H | A-1 | 4-fluorophenyl | 112° C. |
| I-151 | I.G | OCH₃ | H | H | A-1 | 2-trifluoromethyl-thiazol-4-yl | 3.55 min |
| I-152 | I.J | OCH₃ | H | H | A-1 | 3-chloro-5-trifluoromethyl- | 127-131° C. |
| | | | | | | pyridin-2-yl | |
| I-153 | I.B | OCH₃ | H | H | A-1 | 3-chloro-5-trifluoromethyl- | 3.45 min |
| | | | | | | pyridin-2-yl | |
| I-154 | I.B | OCH₃ | H | H | A-1 | 5-trifluoromethyl-pyridin-2-yl | 140° C. |
| I-155 | I.A | OCH₃ | H | H | A-1 | 4,6-dimethoxypyrimidin-2-yl | 131-132° C. |
| I-156 | I.F | OCH₃ | H | H | A-1 | pyridin-2-yl | 120-123° C. |
| I-157 | I.A | H | H | H | A-8 | 2-methyl-5-trifluoromethyl-2H- | 2.58 min |
| | | | | | | pyrazol-3-yl | |
| I-158 | I.A | OCH₃ | H | H | A-1 | 5-difluoromethoxy-pyridin- | 3.21 min |
| | | | | | | 2-yl | |
| I-159 | I.E | H | H | H | A-1 | 3-chloro-5-trifluoromethyl- | 194-196° C. |
| | | | | | | pyridin-2-yl | |
| I-160 | I.F | OCH₃ | H | H | A-1 | 3-chloro-5-trifluoromethyl- | 127-135° C. |
| | | | | | | pyridin-2-yl | |
| I-161 | I.G | OCH₃ | H | H | A-1 | pyrimidin-2-yl | 96° C. |
| I-162 | I.A | OCH₃ | H | H | A-21 | 3-chloro-5-trifluoromethyl- | 57° C. |
| | | | | | | pyridin-2-yl | |
| I-163 | I.A | OCH₃ | H | H | A-26 | 5-trifluoromethyl-pyridin-2-yl | 106° C. |
| I-164 | I.A | OCH₃ | H | H | A-25 | 3-chloro-5-trifluoromethyl- | 3.77 min |
| | | | | | | pyridin-2-yl | |
| I-165 | I.A | OCH₃ | H | H | A-25 | 5-trifluoromethyl-pyridin-2-yl | |
| I-166 | I.A | OCH₃ | H | H | A-1 | 5-methylsulfanyl-pyridin-2-yl | 101-105° C. |
| I-167 | I.A | OCH₃ | H | H | A-2 | 2-trifluoromethyl-pyridin-4-yl | 2.59 min |
| I-168 | I.A | OCH₃ | H | H | A-3 | 2-trifluoromethyl-pyridin-4-yl | 3.06 min |
| I-169 | I.K | OCH₃ | H | H | A-1 | 5-trifluoromethyl-pyridin-2-yl | 176° C. |
| I-170 | I.A | OCH₃ | H | H | A-2 | 5-(chloro-difluoro-methyl)- | 3.45 min |
| | | | | | | pyridin-2-yl | |
| I-171 | I.A | OCH₃ | H | H | A- | 6-bromo-pyridin-3-yl | 172° C. |
| | | | | | 16 | | |
| I-172 | I.A | OCH₃ | H | H | A-1 | 6-dimethylamino-pyridin-3-yl | 130° C. |
| I-173 | I.K | OCH₃ | H | H | A-1 | 3-chloro-5-trifluoromethyl- | 177° C. |
| | | | | | | pyridin-2-yl | |
| I-174 | I.A | OCH₃ | H | H | A-1 | 2-trifluoromethyl-pyridin-4-yl | 129° C. |
| I-175 | I.A | OCH₃ | H | H | A-2 | 2-trifluoromethyl-pyridin-4-yl | |
| I-176 | I.A | OCH₃ | H | H | A-3 | 2-trifluoromethyl-pyridin-4-yl | |
| I-177 | I.A | OCH₃ | H | H | A-3 | 3-fluoro-pyridin-4-yl | |
| I-178 | I.A | OCH₃ | H | H | A-2 | 3-fluoro-pyridin-4-yl | 143-146° C. |
| I-179 | I.A | OCH₃ | H | H | A-1 | 7-chloro-quinolin-4-yl | 142-146° C. |
| I-180 | I.A | OCH₃ | H | H | A-1 | 7-trifluoromethyl-quinolin-4-yl | 140-149° C. |
| I-181 | I.A | OCH₃ | H | H | A-1 | 6-fluoro-2-trifluromethyl- | 198-201° C. |
| | | | | | | quinolin-4-yl | |
| I-182 | I.A | OCH₃ | H | H | A-1 | 2-methyl-3-chloro-quinolin-4- | 202-205° C. |
| | | | | | | yl | |
| I-183 | I.A | OCH₃ | H | H | A-1 | 3,5-dichloro-pyridin-4-yl | 102-205° C. |
| I-184 | I.A | OCH₃ | H | H | A-2 | 3-bromo-pyridin-4-yl | |
| I-185 | I.A | OCH₃ | H | H | A-3 | 3-bromo-pyridin-4-yl | 167° C. |

*Formula selected from I.A to I.K as defined earlier herein;
**A has one of the definitions A-1 to A-26 as described earlier herein;
m.p. = melting point;
Rₜ = HPLC retention time in min: HPLC column: RP-18 column (Chromolith
Speed ROD from Merck KgaA, Germany), 50 mm × 4.6 mm; Eluent:
acetonitrile + 0.1% trifluoroacetic acid (TFA)/water + 0.1% TFA
(gradient from 5:95 to 95:5 in 5 min at 40° C., flow of 1.8 ml/min;
MS: Quadrupol Elektrospray Ionisation, 80 V (positive mode)

TABLE I-b
Compounds of formula I.1.

| ex. | | | | | | | | m.p. [° C.]; |
|---|---|---|---|---|---|---|---|---|
| no | R | R^{a1} | R^{a2} | R^{a3} | A** | Y | Het | Rₜ [min] |
| Ib-1 | C₂H₅ | OCH₃ | H | H | A-3 | —O— | 5-trifluoromethyl-pyridin-2-yl | 3.77 min |
| Ib-2 | CH₃ | OCH₃ | H | H | A-3 | —O— | 5-trifluoromethyl-pyridin-2-yl | |
| Ib-3 | Benzyl | OCH₃ | H | H | A-3 | —O— | 5-trifluoromethyl-pyridin-2-yl | 4.11 min |
| Ib-4 | Allyl | OCH₃ | H | H | A-3 | —O— | 5-trifluoromethyl-pyridin-2-yl | 3.86 min |

Legend as described for Table I-a.

### III. Examples of the Action Against Harmful Fungi

### III.A Glasshouse Trials

The active compounds were formulated separately or together as a stock solution comprising 25 mg of active compound which was made up to 10 ml using a mixture of acetone and/or dimethyl sulfoxide (DMSO) and the emulsifier Uniperol® EL (wetting agent having emulsifying and dispersing action based on ethoxylated alkylphenols) in a volume ratio of solvent/emulsifier of 99:1. This solution was then made up to 100 ml using water. This stock solution was diluted with the solvent/emulsifier/water mixture described to the active compound concentration given below.

### Use Example 1

### Protective Action Against Early Blight on Tomatoes Caused by Phytophthora Infestans

Young seedlings of tomato plants were grown in pots. The plants were sprayed to runoff with an aqueous suspension containing the concentration of active ingredient stated below. The next day, the treated plants were inoculated with an aqueous suspension of sporangia of Phytophthora infestans. After inoculation, the trial plants were immediately transferred to a humid chamber. After 6 days at 18 to 20° C. and a relative humidity close to 100%, the extent of fungal attack on the leaves was visually assessed as % diseased leaf area.

In this test, the plants which had been treated with 250 ppm of the active compound from examples I-7, I-8, I-10, I-11, I-13, I-16, I-17, I-19, I-20, I-21, I-23, I-24, I-27, I-30, I-32, I-34, I-36, I-38 and I-39, respectively, showed an infection of less than or equal to 15% whereas the untreated plants were 90% infected.

### Use Example 2

### Protective Action Against Brown Rust on Wheat Caused by Puccinia Recondita

Leaves of potted wheat seedlings of the cultivar “Kanzler” were sprayed to runoff point with an aqueous suspension having the concentration of active compound stated below. The next day, the treated plants were dusted with a suspension of spores of brown rust of wheat (Puccinia recondita). The plants were then placed in a chamber with high atmospheric humidity (90 to 95%), at 20 to 22° C., for 24 hours. During this time, the spores germinated and the germinal tubes penetrated into the leaf tissue. The next day, the test plants were returned into the greenhouse and cultivated at temperatures between 20 and 22° C. and at 65 to 70% relative atmospheric humidity for a further 7 days. The extent of the rust development on the leaves was then determined visually.

In this test, the plants which had been treated with 250 ppm of the active compound from examples I-1, I-2, I-3, I-4, I-5, I-6, I-8, I-9, I-10, I-11, I-12, I-15, I-16, I-17, I-18, I-19, I-20, I-21, I-22, I-23, I-24, I-25, I-26, I-27, I-28, I-29, I-30, I-32, I-34, I-35, I-36, I-37, I-38, respectively, showed an infection of less than or equal to 20% whereas the untreated plants were 90% infected.

### Use Example 3

### Curative Action Against Soybean Rust on Soybeans Caused by Phakopsora Pachyrhizi

Leaves of potted soybean seedlings were dusted with a suspension of spores of soybean rust (Phakopsora pachyrhizi). The plants were then placed in a chamber with high atmospheric humidity (90 to 95%), at 23 to 27° C., for 24 hours. During this time, the spores germinated and the germinal tubes penetrated into the leaf tissue. The next day, the infected plants were sprayed to runoff point with an aqueous suspension having the concentration of active compound stated below. After drying of the sprayed suspension, the test plants were returned to the greenhouse and cultivated at temperatures between 23 and 27° C. and at 60 to 80% relative atmospheric humidity for a further 14 days. The extent of the rust development on the leaves was then determined visually.

In this test, the plants which had been treated with 250 ppm of the active compound from examples 1-2 and 1-15, respectively, showed an infection of less than or equal to 15% whereas the untreated plants were 90% infected.

### III.B Mitcrotiter Tests

The active substances were formulated separately as a stock solution in dimethyl sulfoxide (DMSO) at a concentration of 10 000 ppm.

The stock solutions were mixed according to the ratio, pipetted onto a micro titer plate (MTP) and diluted with water to the stated concentrations. A spore suspension of the respective fungus in an aqueous medium solution containing yeast extract, bactopeptone and glycerol was then added. The plates were placed in a water vapor-saturated chamber at a temperature of 18° C. Using an absorption photometer, the MTPs were measured at 405 nm 7 days after the inoculation.

The measured parameters were compared to the growth of the active compound-free control variant (100%) and the fungus-free and active compound-free blank value to determine the relative growth in % of the pathogens in the respective active compounds. These percentages were converted into efficacies. An efficacy of 0 means that the growth level of the pathogens corresponds to that of the untreated control; an efficacy of 100 means that the pathogens were not growing.

### Use Example 4

### Activity Against the Late Blight Pathogen Phytophthora Infestans

In this case, a pea-juice based aqueous nutrient medium was used instead of the medium solution containing yeast extract, bactopeptone and glycerol.

In this test, the sample which had been treated with 125 ppm of the active compound from examples I-22, I-27, I-37, I-47, I-48, I-52, I-72, I-76, I-77, I-83, I-88, I-110, I-111, I-112, I-118, I-125, I-128, I-130, I-134, I-144, I-149, I-155, I-159, I-161, I-167, I-171, I-172 and I-173, respectively, showed up at most 15% growth of the pathogen.

### Use Example 5

### Activity Against the Sheath Blight Pathogen Pyricularia Oryzae

In this test, the sample which had been treated with 125 ppm of the active compound from examples I-22, I-37, I-47, I-48, I-52, I-72, I-77, I-83, I-88, I-110, I-112, I-118, I-125, I-134, I-155, I-159, I-161, I-167, I-172 and I-173, respectively, showed up at most 16% growth of the pathogen.

### Use Example 6

### Activity Against Leaf Blotch Pathogen Septoria Tritici

In this test, the sample which had been treated with 125 ppm of the active compound from examples I-22, I-37, I-72, I-77 and I-83, respectively, showed up at most 15% growth of the pathogen.

### Use Example 7

### Activity Against Leptosphaeria Nodorum

In this test, the sample which had been treated with 125 ppm of the active compound from examples I-22, I-37, I-72, I-77, I-88, I-134 and I-173, respectively, showed up at most 20% growth of the pathogen.

### Use Example 8

### Activity Against Ustilago Maydis

In this test, the sample which had been treated with 125 ppm of the active compound from examples I-22, I-134, I-167 and I-173, respectively, showed up at most 10% growth of the pathogen.

### Use Example 9

### Activity Against Septoria Glycines

In this test, the sample which had been treated with 125 ppm of the active comopund from examples I-37, I-76, I-77, I-83, I-88, I-112, I-134, I-159, I-161, I-167 and I-173, respectively, showed up at most 16% growth of the pathogen.

### Use Example 10

### Activity Against Sclerotinia Sclerotiorum

In this test, the sample which had been treated with 125 ppm of the active compound from examples I-37, I-48, I-52, I-72, I-77, I-83, I-88, I-110, I-112, I-118, I-125, I-134, I-149, I-159, I-161, I-167, I-172 and I-173, respectively, showed up at most 17% growth of the pathogen.

### Use Example 11

### Activity Against Cercospora Sojina

In this test, the sample which had been treated with 125 ppm of the active compound from examples I-37, I-77 and I-88, respectively, showed up at most 17% growth of the pathogen.

### Use Example 12

### Activity Against Gaeumannomyces Graminis

In this test, the sample which had been treated with 125 ppm of the active compound from examples I-22, I-37, I-48, I-52, I-72, I-77, I-83, I-88, I-110, I-112, I-125, I-159, I-161, I-167 and I-173, respectively, showed up at most 17% growth of the pathogen.

### Use Example 13

### Activity Against Thielaviopsis Basicola

In this test, the sample which had been treated with 125 ppm of the active compound from examples I-22, I-77, I-83, I-88, I-112, I-134, I-155, I-159, I-172 and I-173, respectively, showed up at most 17% growth of the pathogen.

### IV. Synergistic Mixture Examples

### IV.A Microtiter Tests

These tests were carried out as described above (see III.B), but with the exception of use example 17 an aqueous biomalt solution was used instead of the medium solution containing yeast extract, bactopeptone and glycerol.

The products pyraclostrobin, epoxiconazole and boscalid were used as commercial finished formulations and diluted with water to the stated concentration of the active compound.

The expected efficacies of active compound mixtures were determined using Colby's formula [R. S. Colby, Calculating synergistic and antagonistic responses of herbicide combinations, Weeds 15, 20-22 (1967)] and compared with the observed efficacies.

Colby's formula: E=x+y−x·y/100
E expected efficacy, expressed in % of the untreated control, when using the mixture of the compounds A and B at the concentration a and b
   x efficacy, expressed in % of the untreated control, when using compound A at a concentration of a
   y efficacy, expressed in % of the untreated control, when using compound B at a concentration of b

### Use Example 14

### Activity Against Leaf Blotch on Wheat Caused by Septoria Tritici

TABLE II

| | Concentration | | | |
|---|---|---|---|---|
| Compound or | of compounds | Mixing | Observed | Expected |
| mixture tested | (ppm) | ratio | efficacy (%) | efficacy (%) |
| Ex. No. I-5 | 4 | n.a. | 22 | n.a. |
| Ex. No. I-27 | 4 | n.a. | 27 | n.a. |
| Ex. No. I-37 | 4 | n.a. | 20 | n.a. |
| Ex. No. I-52 | 4 | n.a. | 29 | n.a. |
| Ex. No. I-72 | 4 | n.a. | 14 | n.a. |
| Ex. No. I-118 | 4 | n.a. | 25 | n.a. |
| Ex. No. I-125 | 4 | n.a. | 23 | n.a. |
| Pyraclostrobin | 0.063 | n.a. | 74 | n.a. |
| Boscalid | 4 | n.a. | 75 | n.a. |
| Ex. No. I-5 + | 4 | 64:1 | 99 | 80 |
| Pyraclostrobin | 0.063 | | | |
| Ex. No. I-27 + | 4 | 64:1 | 99 | 81 |
| Pyraclostrobin | 0.063 | | | |
| Ex. No. I-37 + | 4 | 64:1 | 98 | 80 |
| Pyraclostrobin | 0.063 | | | |
| Ex. No. I-52 + | 4 | 64:1 | 100 | 82 |
| Pyraclostrobin | 0.063 | | | |
| Ex. No. I-118 + | 4 | 64:1 | 99 | 81 |
| Pyraclostrobin | 0.063 | | | |
| Ex. No. I-125 + | 4 | 64:1 | 100 | 80 |
| Pyraclostrobin | 0.063 | | | |
| Ex. No. I-37 + | 4 | 1:1 | 100 | 80 |
| Boscalid | 4 | | | |
| Ex. No. I-72 + | 4 | 1:1 | 98 | 79 |
| Boscalid | 4 | | | |
| Ex. No. I-118 + | 4 | 1:1 | 99 | 81 |
| Boscalid | 4 | | | |
| Ex. No. I-125 + | 4 | 1:1 | 100 | 81 |
| Boscalid | 4 | | | |

n.a. = not applicable

### Use Example 15

### Activity Against Alternaria Solani

TABLE III

| | Concentration | | | |
|---|---|---|---|---|
| Compound or | of compounds | Mixing | Observed | Expected |
| mixture tested | (ppm) | ratio | efficacy (%) | efficacy (%) |
| Ex. No. I-5 | 0.25 | n.a. | 2 | n.a. |
| Ex. No. I-22 | 0.25 | n.a. | 3 | n.a. |
| Ex. No. I-27 | 0.25 | n.a. | 3 | n.a. |
| | 4 | n.a. | 0 | n.a. |
| Ex. No. I-37 | 0.25 | n.a. | 5 | n.a. |
| | 4 | n.a. | 3 | n.a. |
| Ex. No. I-47 | 0.25 | n.a. | 1 | n.a. |
| Ex. No. I-52 | 0.25 | n.a. | 0 | n.a. |
| | 4 | n.a. | 0 | n.a. |
| Ex. No. I-72 | 0.25 | n.a. | 3 | n.a. |
| Ex. No. I-88 | 0.25 | n.a. | 4 | n.a. |
| Ex. No. I-118 | 0.25 | n.a. | 1 | n.a. |
| Ex. No. I-125 | 0.25 | n.a. | 4 | n.a. |
| Pyraclostrobin | 0.063 | n.a. | 48 | n.a. |
| Boscalid | 0.25 | n.a. | 49 | n.a. |
| Ex. No. I-27 + | 4 | 64:1 | 73 | 48 |
| Pyraclostrobin | 0.063 | | | |
| Ex. No. I-37 + | 4 | 64:1 | 68 | 48 |
| Pyraclostrobin | 0.063 | | | |
| Ex. No. I-52 + | 4 | 64:1 | 69 | 48 |
| Pyraclostrobin | 0.063 | | | |
| Ex. No. I-5 + | 0.25 | 1:1 | 74 | 50 |
| Boscalid | 0.25 | | | |
| Ex. No. I-22 + | 0.25 | 1:1 | 75 | 51 |
| Boscalid | 0.25 | | | |
| Ex. No. I-27 + | 0.25 | 1:1 | 76 | 51 |
| Boscalid | 0.25 | | | |
| Ex. No. I-37 + | 0.25 | 1:1 | 76 | 52 |
| Boscalid | 0.25 | | | |
| Ex. No. I-47 + | 0.25 | 1:1 | 73 | 49 |
| Boscalid | 0.25 | | | |
| Ex. No. I-52 + | 0.25 | 1:1 | 77 | 49 |
| Boscalid | 0.25 | | | |
| Ex. No. I-72 + | 0.25 | 1:1 | 78 | 51 |
| Boscalid | 0.25 | | | |
| Ex. No. I-88 + | 0.25 | 1:1 | 78 | 51 |
| Boscalid | 0.25 | | | |
| Ex. No. I-118 + | 0.25 | 1:1 | 77 | 49 |
| Boscalid | 0.25 | | | |
| Ex. No. I-125 + | 0.25 | 1:1 | 81 | 51 |
| Boscalid | 0.25 | | | |

n.a. = not applicable

### Use Example 16

### Activity Against Pyrenophora Teres

TABLE IV

| | Concentration | | | |
|---|---|---|---|---|
| Compound or | of compounds | Mixing | Observed | Expected |
| mixture tested | (ppm) | ratio | efficacy (%) | efficacy (%) |
| Ex. No. I-5 | 4 | n.a. | 21 | n.a. |
| | 0.25 | n.a. | 0 | n.a. |
| Ex. No. I-22 | 0.016 | n.a. | 1 | n.a. |
| Ex. No. I-27 | 4 | n.a. | 26 | n.a. |
| Ex. No. I-37 | 4 | n.a. | 18 | n.a. |
| | 0.25 | n.a. | 0 | n.a. |
| Ex. No. I-72 | 4 | n.a. | 14 | n.a. |
| Ex. No. I-88 | 0.25 | n.a. | 16 | n.a. |
| Epoxiconazole | 0.25 | n.a. | 8 | n.a. |
| Pyraclostrobin | 0.004 | n.a. | 0 | n.a. |
| Boscalid | 0.25 | n.a. | 63 | n.a. |
| | 0.016 | n.a. | 0 | n.a. |
| Ex. No. I-5 + | 4 | 16:1 | 45 | 27 |
| Epoxiconazole | 0.25 | | | |
| Ex. No. I-27 + | 4 | 16:1 | 52 | 32 |
| Epoxiconazole | 0.25 | | | |
| Ex. No. I-37 + | 4 | 16:1 | 61 | 25 |
| Epoxiconazole | 0.25 | | | |
| Ex. No. I-72 + | 4 | 16:1 | 62 | 21 |
| Epoxiconazole | 0.25 | | | |
| Ex. No. I-88 + | 0.25 | 64:1 | 39 | 16 |
| Pyraclostrobin | 0.004 | | | |
| Ex. No. I-5 + | 0.25 | 1:1 | 84 | 63 |
| Boscalid | 0.25 | | | |
| Ex. No. I-22 + | 0.016 | 1:1 | 29 | 1 |
| Boscalid | 0.016 | | | |
| Ex. No. I-37 + | 0.25 | 1:1 | 85 | 63 |
| Boscalid | 0.25 | | | |

n.a. = not applicable

### Use Example 17

### Activity Against the Late Blight Pathogen Phytophthora Infestans

In this case, a pea-juice based aqueous nutrient medium was used instead of the medium solution containing yeast extract, bactopeptone and glycerol.

TABLE V

| | Concentration | | | |
|---|---|---|---|---|
| Compound or | of compounds | Mixing | Observed | Expected |
| mixture tested | (ppm) | ratio | efficacy (%) | efficacy (%) |
| Ex. No. I-52 | 16 | n.a. | 53 | n.a. |
| Pyraclostrobin | 0.25 | n.a. | 38 | n.a. |
| Ex. No. I-52 + | 16 | 64:1 | 90 | 71 |
| Pyraclostrobin | 0.25 | | | |

n.a. = not applicable

### IV.B Glasshouse Trials

The spray solutions were prepared in several steps: The stock solution were prepared: a mixture of acetone and/or dimethylsulfoxide and the wetting agent/emulsifier Wettol, which is based on ethoxylated alkylphenoles, in a relation (volume) solvent-emulsifier of 99 to 1 was added to 25 mg of the compound to give a total of 10 ml. Water was then added to total volume of 100 ml. This stock solution was diluted with the described solvent-emulsifier-water mixture to the given concentration.

The products pyraclostrobin, epoxiconazole and boscalid were used as commercial finished formulations and diluted with water to the stated concentration of the active compound.

### Use Example 18

### Preventative Control of Brown Rust Caused by Puccinia Recondita

The first two developed leaves of pot-grown wheat seedling were sprayed to run-off with an aqueous suspension, containing the concentration of active ingredient or their mixture as described below. The next day the plants were inoculated with spores of Puccinia recondita. To ensure the success the artificial inoculation, the plants were transferred to a humid chamber without light and a relative humidity of 95 to 99% and 20 to 22° C. for 24 h. Then the trial plants were cultivated for 6 days in a greenhouse chamber at 22-26° C. and a relative humidity between 65 and 70%. The extent of fungal attack on the leaves was visually assessed as % diseased leaf area.

The percentages diseased leaf area were converted into efficacies. An efficacy of 0 means that the infection level of the treated plants corresponds to that of the untreated control plants; an efficacy of 100 means that the treated plants were not infected.

The expected efficacies of active compound mixtures were determined using Colby's formula as described earlier herein.

TABLE VI

| | Concentration | | | |
|---|---|---|---|---|
| Compound or | of compounds | Mixing | Observed | Expected |
| mixture tested | (ppm) | ratio | efficacy (%) | efficacy (%) |
| untreated | n.a. | n.a. | (80% | n.a. |
| control | | | diseased | |
| | | | leaf area) | |
| Ex. No. I-27 | 16 | n.a. | 27 | n.a. |
| Ex. No. I-37 | 4 | n.a. | 20 | n.a. |
| Ex. No. I-47 | 4 | n.a. | 29 | n.a. |
| Ex. No. I-52 | 16 | n.a. | 29 | n.a. |
| Ex. No. I-72 | 16 | n.a. | 0 | n.a. |
| | 4 | n.a. | 0 | n.a. |
| Ex. No. I-125 | 4 | n.a. | 13 | n.a. |
| Pyraclostrobin | 0.25 | n.a. | 0 | n.a. |
| Boscalid | 16 | n.a. | 0 | n.a. |
| | 4 | n.a. | 0 | n.a. |
| Epoxiconazole | 0.25 | n.a. | 0 | n.a. |
| Ex. No. I-27 + | 16 | 64:1 | 64 | 29 |
| Pyraclostrobin | 0.25 | | | |
| Ex. No. I-52 + | 16 | 64:1 | 75 | 38 |
| Pyraclostrobin | 0.25 | | | |
| Ex. No. I-37 + | 4 | 1:1 | 57 | 29 |
| Boscalid | 4 | | | |
| Ex. No. I-47 + | 4 | 1:1 | 29 | 0 |
| Boscalid | 4 | | | |
| Ex. No. I-52 + | 16 | 1:1 | 63 | 38 |
| Boscalid | 16 | | | |
| Ex. No. I-72 + | 16 | 1:1 | 25 | 0 |
| Boscalid | 16 | 1:1 | | |
| Ex. No. I-47 + | 4 | 16:1 | 29 | 0 |
| Epoxiconazole | 0.25 | | | |
| Ex. No. I-72 + | 4 | 16:1 | 25 | 0 |
| Epoxiconazole | 0.25 | | | |
| Ex. No. I-125 + | 4 | 16:1 | 50 | 13 |
| Epoxiconazole | 0.25 | | | |

n.a. = not applicable

We claim:

1-15. (canceled)

16. A pyrimidylmethyl-sulfonamide compound of formula I 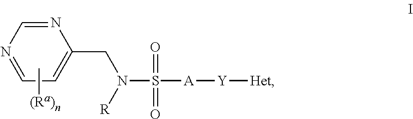
wherein:
n indicates the number of substituents R^{a} on the pyrimidine ring and n is 0, 1, 2 or 3;
R^{a} is halogen, CN, NH₂, NO₂, OH, SH, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylamino, di(C₁-C₄-alkyl)amino, C₁-C₄-alkoxy-C₁-C₄-alkyl, C₂-C₄-alkenyl, C₂-C₄-alkynyl, C₃-C₈-cycloalkyl or C₁-C₄-alkyl-C₃-C₈-cycloalkyl; or
two radicals R^{a} that are bound to adjacent ring member atoms of the pyrimidine ring may form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of consisting of N, O and S, and wherein the fused carbocycle or heterocycle is unsubstituted or carries 1, 2, 3 or 4 identical or different radicals selected from the group consisting of halogen, CN, C₁-C₄-alkyl, C₁-C₄alkoxy, C₁-C₄-haloalkyl and C₁-C₄-haloalkoxy;
it being possible for n=2 or 3 that R^{a} are identical or different;
R is hydrogen, C₁-C₄-alkyl, C₁-C₄-haloalkyl, C₁-C₄-alkoxy, C₁-C₄-haloalkoxy, C₁-C₄-alkylamino, di(C₁-C₄-alkyl)amino, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₃-C₈-cycloalkyl, C₁-C₄-alkyl-C₃-C₈-cycloalkyl or benzyl wherein the phenyl moiety of benzyl is unsubstituted or carries 1, 2, 3, 4, or 5 substituents selected from the group consisting of cyano, halogen, C₁-C₄-alkyl, C₁-C₄-haloalkyl, C₁-C₄-alkoxy, C₁-C₄-haloalkoxy, (C₁-C₄-alkoxy)carbonyl and di(C₁-C₄-alkyl)aminocarbonyl,
A is phenylene or a 5- or 6-membered heteroarenediyl, wherein the ring member atoms of the heteroarenediyl include besides carbon atoms 1, 2, 3 or 4 heteroatoms selected from the group of consisting of N, O and S, and wherein the aforementioned divalent radicals are unsubstituted or carry 1, 2, 3 or 4 identical or different groups R^{b}:
R^{b} is halogen, CN, NO₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, (C₁-C₄-alkyl)carbonyl, (C₁-C₄-alkoxy)carbonyl, C₁-C₄-alkylamino, di(C₁-C₄-alkyl)amino, (C₁-C₄-alkyl)aminocarbonyl and di(C₁-C₄-alkyl)aminocarbonyl;
Y is a divalent group selected from the group consisting of —O—, —O—CH₂—, —CH₂—O—, —S—, —S(═O)—, —S(═O)₂—, C₁-C₄-alkanediyl, —N(R^{π})— and —C(NOR^{π})—, wherein R^{π} is hydrogen or C₁-C₄-alkyl;
Het is a 5- or 6-membered heteroaryl, wherein the ring member atoms of the heteroaryl include besides carbon atoms 1, 2, 3 or 4 heteroatoms selected from the group consisting of N, O and S and wherein the heteroaryl is unsubstituted or carries 1, 2, 3 or 4 identical or different groups R^{c}:
R^{c} is halogen, CN, NO₂, NH₂, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C(═O)R′, C(═NOR″)R′″, C₃-C₈-cycloalkyl, C₁-C₄-alkyl-C₃-C₈-cycloalkyl, phenyl, phenoxy, phenoxy-C₁-C₄-alkyl or a 5- or 6-membered heteroaryl, wherein the ring member atoms of the heteroaryl include besides carbon atoms 1, 2, 3 or 4 heteroatoms selected from the group consisting of N, O and S, and wherein the aforementioned cyclic radicals are unsubstituted or carry 1, 2, 3 or 4 identical or different substituents R^{d}:
R′ is hydrogen, NH₂, C₁-C₄-alkyl, C₁-C₄-haloalkyl, C₂-C₄-alkenyl, C₂-C₄-alkynyl, C₁-C₄-alkoxy, C₁-C₄-alkoxy-C₁-C₄-alkoxy, C₁-C₄-haloalkoxy, C₁-C₄-alkylamino or di(C₁-C₄-alkyl)amino;
R″ is hydrogen, C₁-C₄-alkyl, C₁-C₄-haloalkyl, C₂-C₄-alkenyl, C₂-C₄-alkynyl or C₁-C₄-alkoxy-C₁-C₄-alkyl,
R′″ is hydrogen or C₁-C₄-alkyl;
R^{d} is halogen, CN, C₁-C₄-alkyl, C₁-C₄-haloalkyl, C₁-C₄-alkoxy or C₁-C₄-haloalkoxy;
or two radicals R^{c} that are bound to adjacent ring member atoms of the Het group may form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group consisting of N, O and S, and wherein the fused carbocycle or heterocycle is unsubstituted or carries 1, 2, 3 or 4 identical or different radicals groups R^{e}:
R^{e} is halogen, CN, C₁-C₄-alkyl, C₁-C₄-haloalkyl, C₁-C₄-alkoxy or C₁-C₄-haloalkoxy;
and/or the N-oxide and the agriculturally acceptable salt of the compound of formula I, and of a composition comprising a compound of formula I, for combating phytopathogenic fungi.

17. A compound according to claim 16, wherein the pyrmidin-4-yl moiety 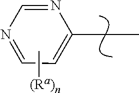
is selected from the group consisting of pyrimidin-4-yl, 2-methylpyrimidin-4-yl, 3-methylpyrimidin-4-yl, 2-ethylpyrimidin-4-yl, 3-ethylpryrid-4-yl, 2,3-dimethylpyrimidin-4-yl, 2,3-diethylpyrimidin-4-yl, 2-methoxypyrimidin-4-yl, 3-methoxypyrimidin-4-yl, 2-difluoromethoxypyrimidin-4-yl, 2-cyanopyrimidin-4-yl, 2-chloropyrimidin-4-yl, 2-bromopyrimidin-4-yl, 2-chloro-3-methylpyrimidin-4-yl, 3-chloro-2-methylpyrimidin-4-yl, 2-chloro-3-ethylpyrimidin-4-yl, 3-chloro-2-ethylpyrimidin-4-yl, 2-methoxy-3-methylpyrimidin-4-yl and 3-methoxy-2-methylpyrimidin-4-yl.

18. A compound according to claim 16, wherein Het is pyrimidin-2-yl, pyrimidin-3-yl, pyrimidin-4-yl, pyridin-2-yl, pyridin-3-yl, pyrazin-2-yl, pyridazin-3-yl, 1,3,5-triazin-2-yl, or 1,2,4-triazin-3-yl, where the aforementioned heteroaromatic radicals are unsubstituted or carry 1, 2 or 3 identical or different substituents R^{c}.

19. A compound according to claim 16, wherein Het carries 1 or 2 radicals R^{e} which are selected from the group consisting of F, Cl, Br, CN, C₁-C₂-alkylsulfonyl, C₁-C₂-alkoxycarbonyl, aminocarbonyl, C₁-C₂-alkylaminocarbonyl, di(C₁-C₂-alkyl)aminocarbonyl, C₁-C₂-alkoxy, CF₃, CHF₂, OCF₃ and OCHF₂.

20. A compound according to claim 16, wherein R is hydrogen.

21. A compound according to claim 16, wherein Y is —O—.

22. A compound according to claim 16, wherein A is 1,4-phenylene, which is unsubstituted or carries 1, 2, 3 or 4 identical or different substituents R^{b}.

23. A process for preparing the compound of claim 16, which comprises reacting an aminomethylpyrimidine compound of formula II 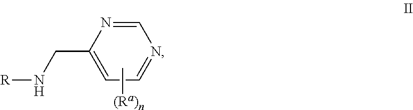
wherein n, R^{a} and R are as defined in claim 16, under basic conditions with a sulfonic acid derivative of formula III 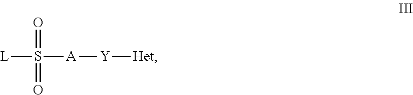
wherein A, Y and Het are as defined in claim 16 and L is a leaving group selected from the group consisting of chloro, fluoro, azido, optionally substituted heteroaryl, optionally substituted heteroaryloxy and optionally substituted phenoxy, wherein the heteroaryl radical is selected from the group consisting of pyrazol-1-yl, imidazol-1-yl, 1,2,3-triazol-1-yl and 1,2,4-triazol-1-yl, and wherein the heteroaryl, heteroaryloxy and phenoxy radicals are unsubstituted or carry one, two, three, four or five identical or different substituents selected from the group consisting of halogen, C₁-C₄-alkyl and C₁-C₄-haloalkyl, and/or two substituents that are bound to adjacent ring member atoms of the heteroaryl, heteroaryloxy and phenoxy radicals may form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group consisting of N, O and S, and wherein the fused carbocycle or heterocycle is unsubstituted or carries one, two, three or four identical or different substituents selected from the group consisting of halogen, C₁-C₄-alkyl and C₁-C₄-haloalkyl.

24. An intermediate compound IX.a 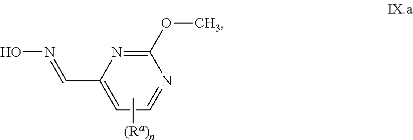
wherein
R^{a} is halogen, CN, NH₂, NO₂, OH, SH, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylamino, di(C₁-C₄-alkyl)amino, C₁-C₄-alkoxy-C₁-C₄-alkyl, C₂-C₄-alkenyl, C₂-C₄-alkynyl, C₃-C₈-cycloalkyl or C₁-C₄-alkyl-C₃-C₈-cycloalkyl; or
two radicals R^{a} that are bound to adjacent ring member atoms of the pyrimidine ring may form together with said ring member atoms a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group consisting of N, O and S, and wherein the fused carbocycle or heterocycle is unsubstituted or carries 1, 2, 3 or 4 identical or different radicals selected from the group consisting of halogen, CN, C₁-C₄-alkoxy, C₁-C₄-haloalkyl and C₁-C₄-haloalkoxy;
it being possible for n=2 or 3 that R^{a} are identical or different;
R is hydrogen, C₁-C₄-alkyl, C₁-C₄-alkoxy, C₁-C₄-haloalkoxy, C₁-C₄-alkylamino, di(C₁-C₄alkyl)amino, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₃-C₈-cycloalkyl, C₁-C₄-alkyl-C₃-C₈-cycloalkyl or benzyl wherein the phenyl moiety of benzyl is unsubstituted or carries 1, 2 , 3, 4, or 5 substituents selected from the group consisting of cyano, halogen, C₁-C₄-alkyl, C₁-C₄-haloalkyl, C₁-C₄-alkoxy, C₁-C₄-haloalkoxy, (C₁-C₄-alkoxy)carbonyl and di(C₁-C₄-alkyl)aminocarbonyl,
and n is 0, 1 or 2.

25. An agrochemical composition which comprises a solid or liquid carrier and at least one compound of claim 16 or an N-oxide or an agriculturally acceptable salt thereof

26. The agrochemical composition according to claim 25 comprising at least one further active substance.

27. A method for combating phytopathogenic harmful fungi, which process comprises treating the fungi or the materials, plants, the soil or seeds to be protected against fungal attack, with an effective amount of at least one compound of claim 16 or an N-oxide or an agriculturally acceptable salt thereof

28. A seed comprising the compound of formula I, according to claim 16 or an N-oxide or an agriculturally acceptable salt thereof, in an amount of from 0.1 g to 10 kg per 100 kg of seed.

29. A method for protecting a seed or a seedlings' roots or shoots from infestation by harmful fungi, which process comprises treating the seed with an effective amount of at least one compound of claim 16 or an N-oxide or an agriculturally acceptable salt thereof.
